(19) United States
(12) Patent Application Publication
(10) Pub. No.: US 2012/0184562 A1
(43) Pub. Date: Jul. 19, 2012

(54) 1,6- AND 1,8-NAPHTHYRIDINES

(76) Inventor: Kin-Chun Luk, North Caldwell, NJ (US)

(21) Appl. No.: 13/348,668

(22) Filed: Jan. 12, 2012

Related U.S. Application Data
(60) Provisional application No. 61/433,999, filed on Jan. 19, 2011.

(51) Int. Cl. A61K 31/496; A61P 25/00; A61P 35/00; A61P 25/28; C07D 471/04; A61K 31/4375

(52) U.S. Cl. 514/253.04; 546/123; 544/362; 514/300

(57) ABSTRACT

Compounds of formula

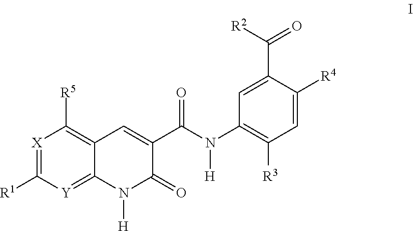

and pharmaceutically acceptable salts thereof are described, as well as the pharmaceutical compositions containing said compounds and their pharmaceutically acceptable salts, and the use of said compounds and pharmaceutical compositions for the treatment, control or amelioration of proliferative diseases, including cancer, Down syndrome or early onset Alzheimer's disease.

### PRIORITY TO RELATED APPLICATIONS

This application claims the benefit of U.S. Provisional Application No. 61/433,999, filed Jan. 19, 2011, which is hereby incorporated by reference in its entirety.

### FIELD OF THE INVENTION

The present invention relates to 1,6 and 1,8-naphthyridines which act as inhibitors of DYRK1B and/or DYRK1A and are useful in the amelioration, treatment or control of cancer, especially solid tumors, or in the amelioration, treatment or control of Down syndrome or early onset Alzheimer's disease.

### BACKGROUND OF THE INVENTION

Kinases are known to be important cellular enzymes that regulate cellular functions such as regulating cell division and proliferation. WO 2008/047307. Dual-specificity tyrosine-phosphorylation-regulated kinases (DYRKs) are a subfamily of protein kinases that have dual-specificity and are believed to play roles in cell proliferation and apoptosis induction. See, e.g., Kiyotsugu Yoshida, “Role for DYRK family kinases on regulation of apoptosis,” Biochemical Pharmacology 76 (2008) pp 1389-1394; Jinghun Gao et al., “Mirk/Dyrk1B, a novel therapeutic target, mediates cells survival in non-small cell lung cancer cells,” Cancer Biology & Therapy 8:17 (2009) pp. 1671-1679. DYRK1A is believed to be implicated in neural differentiation. Yoshida, id. at 1390. Over expression of this kinase is believed to be involved in Down syndrome and Alzheimer's disease. See Nam Kim, “Putative therapeutic agents for learning and memory deficits of people with Down syndrome,” Bioorganic & Medicinal Chemistry Letters,” 16 (2006) pp 3772-76 and Joongkyu Park et al, “Function and regulation of Dyrk1A: towards understanding Down syndrome,” Cell. Mol. Life. Sci 66 (2009) pgs. 3235-3240. Thus, inhibition of this kinase is believed to be of benefit in controlling or ameliorating the effects of Down syndrome and early onset Alzheimer's disease. See, e.g., Kim, id; Park, id, and Kyung Koo et al., “QSAR analysis of pyrazolidine-3,5-diones derivatives as Dyrk1A inhibitors,” Bioorganic & Medicinal Chemistry Letters 19 (2009) pp 2324-2328.

DYRK1B (also referred to as MIRK) mediates survival and differentiation in many tissues. It is believed to be implicated in certain cancers, particularly solid tumors. See, e.g., Gao, supra (lung cancer cells); Kangmoon Lee et al, “Mirk Protein Kinase is a Mitogen-activated Protein Kinase Substrate that Mediates Survival of Colon Cancer Cells”, Cancer Research 60 (2000):3631-3637 and Xiaobing Deng et al, “The Kinase Mirk/Dyrk1B Mediates Cell Survival in a Pancreatic Ductal Adenocarcinoma,” Cancer Res 66:8 (2006) pp 4149-58 (pancreatic cancer cells). Thus, inhibition of this kinase is believed to be of benefit in controlling or ameliorating cancer. See, Cao Yang et al, “The kinase Mirk is a potential therapeutic target in osteosarcoma,” Carcinogenesis 31:4 (2010) pp 552-558 and Eileen Friedman, “The Kinase Mirk/dyrk1B: A Possible Therapeutic Target in Pancreatic Cancer,” Cancers 2 (2010) 1492-1512.

### SUMMARY OF THE INVENTION

One aspect of the invention is a compound of formula I

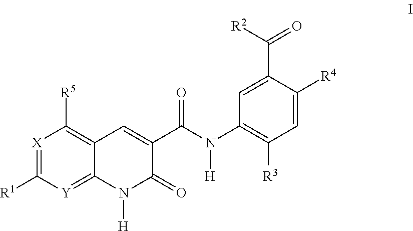

or a pharmaceutically acceptable salt thereof, wherein X, Y and R¹ through R⁵ are as defined below.

The present invention also relates to pharmaceutical compositions comprising one or more compounds of the invention, or a pharmaceutically acceptable salt, and a pharmaceutically acceptable carrier or excipient.

The present invention further relates to a method of treating, ameliorating or controlling cancer, including specifically solid tumors, for example lung, pancreatic, colon, breast, bone and prostate cancers in a mammal, specifically a human, comprising administering to said mammal a therapeutically effective amount of a compound according to the invention or a pharmaceutically acceptable salt thereof.

The present invention further relates to a method of treating, ameliorating or controlling Down syndrome or Alzheimer's disease in a human, comprising administering to said human a therapeutically effective amount of a compound according to the invention or a pharmaceutically acceptable salt thereof.

### DETAILED DESCRIPTION OF THE INVENTION

### Definitions

As used herein, the following terms shall have the following definitions.

The terms “C₁₋₆ alkyl” or “C₁₋₄ alkyl” refer to straight- or branched-chain saturated hydrocarbon groups having from 1 to 6, or 1 to 4, carbon atoms, respectively. Examples of C₁₋₆ alkyl groups include, but are not limited to, methyl, ethyl, n-propyl, i-propyl, n-butyl, s-butyl, t-butyl, n-pentyl, and s-pentyl.

“Alkoxy, alkoxyl or lower alkoxy” refers to any of the above alkyl groups which is attached to the remainder of the molecule by an oxygen atom (RO—). Typical alkoxy groups include methoxy, ethoxy, isopropoxy or propoxy, butyloxy and the like. Further included within the meaning of alkoxy are multiple alkoxy side chains, e.g. ethoxy ethoxy, methoxy ethoxy, methoxy ethoxy ethoxy and the like and substituted alkoxy side chains, e.g., dimethylamino ethoxy, diethylamino ethoxy, dimethoxy-phosphoryl methoxy and the like.

“Aryl” means a substituted or unsubstituted monovalent, monocyclic or bicyclic, aromatic carboxylic hydrocarbon radical, preferably a 6-10 member aromatic ring system. Preferred aryl groups include, but are not limited to, phenyl, naphthyl, tolyl, and xylyl.

The term “cycloalkyl” as used herein means a substituted or unsubstituted stable monocyclic or polycyclic system which consists of carbon atoms only, all rings of which are saturated. Examples of cycloalkyls include, but are not limited to, cyclopropyl, cyclobutyl, cyclopentyl, cyclohexyl, cycloheptyl, adamantyl, cyclooctyl, bicycloalkyls, including bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds.

“Halogen” means Cl, F and Br.

“Heteroaryl” means a substituted or unsubstituted aromatic heterocyclic ring system containing up to two rings. Preferred heteroaryl groups include, but are not limited to, thienyl (or thiophenyl), furyl, indolyl, pyrrolyl, pyridinyl, pyrazinyl, oxazolyl, thiaxolyl, quinolinyl, pyrimidinyl, imidazolyl, triazolyl and tetrazolyl.

In the case of a heteroaryl that is bicyclic it should be understood that one ring may be aryl while the other is heteroaryl and both may be independently substituted or unsubstituted.

“Hetero atom” means an atom selected from N, O and S.

“Heterocycle” or “heterocyclic ring” means a substituted or unsubstituted 5 to 10 membered, mono- or bicyclic, non-aromatic hydrocarbon, wherein 1 to 3 carbon atoms are replaced by a hetero atom selected from nitrogen, oxygen or sulfur atom. Examples include pyrrolidinyl, including pyrrolidin-1-yl, pyrrolidin-2-yl and pyrrolidin-3-yl; piperazinyl; piperidinyl; morpholinyl, including morpholin-4-yl, and the like, each of which optionally can be substituted.

In the case of a heterocycle that is bicyclic it should be understood that one ring may be heterocycle while the other is cycloalkyl, and either or both may be independently substituted. An example of such a bicyclic heterocycle is 8-oxa-3-aza-bicyclo[3.2.1]octane.

Hydroxy or hydroxyl is a prefix indicating the presence of a monovalent —O—H group.

“IC₅₀” refers to the concentration of a particular compound required to inhibit 50% of a specific measured activity. IC₅₀ can be measured, inter alia, as is described subsequently in Examples 102 and 103.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoroacetic acid and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (1995) at pgs. 456-457.

“Substituted,” as in substituted alkyl, means that the substitution can occur at one or more positions and, unless otherwise indicated, that the substituents at each substitution site are independently selected from the specified options. The term “optionally substituted” refers to the fact that one or more hydrogen atoms of a chemical group (with one or more hydrogen atoms) can be, but does not necessarily have to be, substituted with another substituent.

In one embodiment, the present invention relates to compounds of formula I

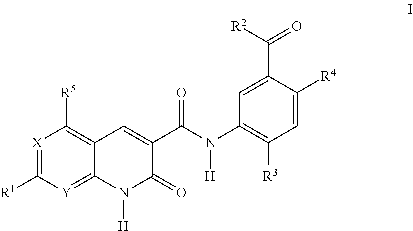

wherein
X and Y are each independently selected from C and N, provided that when one is C and the other is N;
R¹ is selected from the group
(a) H,
(b) C₁₋₄ alkyl,
(c) C₁₋₄ alkyl substituted with up to 3 groups selected from cycloalkyl, heterocycle, OR⁶, NR⁶R⁷, and CN,
(d) OR⁶,
(e) NR⁶R⁷,
(f) heterocycle that is attached to the rest of the molecule via a heteroatom
(g) heterocycle substituted with up to three groups selected from C₁₋₄ alkyl, OR⁸, NR⁸R⁹ and CN, and
(h) SR⁶;
   R² is selected from the group
(a) NR¹⁹R¹¹, and
(b) OR¹²;
   R³ is selected from the group
(a) CH₃,
(b) F,
(c) Cl, and
(d) Br;
   R⁴ is selected from the group
(a) H, and
(b) F;
   R⁵ is selected from the group
(a) H, and
(b) C₁₋₄ alkyl
   R⁶ and R⁷ are independently selected from the group
(a) H,
(b) C₁₋₄ alkyl,
(c) C₁₋₄ alkyl substituted with up to 3 groups selected from OH, OC₁₋₄ alkyl, NR⁸R⁹, CN, heterocycle, and cycloalkyl,
(d) cycloalkyl,
(e) cycloalkyl substituted with up to 3 groups selected from OH, NR⁸R⁹ and C₁₋₄ alkyl,
(f) heterocycle, and
(g) heterocycle substituted with up to three C₁₋₄ alkyl groups;
   R⁸ and R⁹ are independently selected from the group
(a) H, and
(b) C₁₋₄ alkyl;
   R¹⁰ and R¹¹ are independently selected from the group
(a) H,
(b) C₁₋₆ alkyl,
(c) C₁₋₆ alkyl substituted with up to 4 groups selected from
   aryl,
   aryl substituted with Cl, F, or CH₃,
   heteroaryl,
   cycloalkyl,
   heterocycle,
   OH,
   OC₁₋₄ alkyl,
   NR⁸R⁹,
   CN; and
   CONR⁸R⁹, and
(d) aryl optionally substituted with Cl, F or CH₃;
   or alternatively, NR¹⁰R¹¹ together can form a heterocycle that optionally may be substituted with
   Cl,
   F,
   CH₃,
   aryl that optionally may be substituted with Cl, F, and CH₃, and
   heteroaryl that optionally may be substituted with Cl, F, and CH₃; and
      R¹² is selected from the group
(a) C₁₋₆ alkyl,
(b) C₁₋₆ alkyl substituted with up to 4 groups selected from
   aryl,
   aryl substituted with Cl, F, or CH₃,
   heteroaryl,
   cycloalkyl,
   heterocycle,
   OH,
   OC₁₋₄ alkyl,
   NR⁸R⁹,
   CN; and
   CONR⁸R⁹, and
(c) aryl optionally substituted with Cl, F or CH₃;
   or a pharmaceutically acceptable salt thereof.

In another embodiment, the invention relates to compounds of formula Ia having the structure

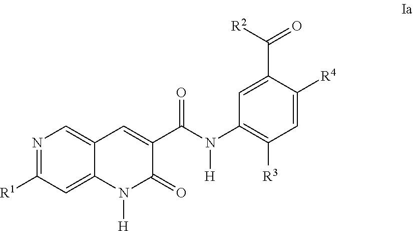

wherein R¹ through R⁴ are as defined above, or a pharmaceutically acceptable salt thereof.

In another embodiment, the invention relates to compounds of formula Ib having the structure

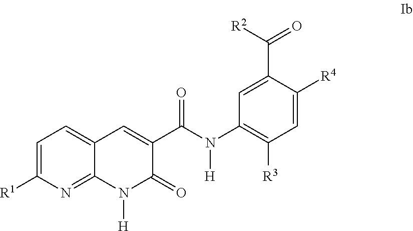

wherein R¹ through R⁴ are as defined above, or a pharmaceutically acceptable salt thereof.

In another embodiment, the invention relates to compounds of formula I, including compounds of formulas Ia and Ib, wherein R¹ is H, or a pharmaceutically acceptable salt thereof.

Another embodiment of the invention relates to compounds of Formula I, including compounds of formulas Ia and Ib, wherein R¹ is OR⁶ and R⁶ is C₁₋₄ alkyl. In one particular embodiment OR⁶ is OCH₃.

Another embodiment of the invention relates to compounds of Formula I, including compounds of formulas Ia and Ib, wherein R¹ is SR⁶ and R⁶ is C₁₋₄ alkyl, or a pharmaceutically acceptable salt thereof. In one embodiment SR⁶ is SCH₃.

Another embodiment of the invention relates to compounds of Formula I, including compounds of formulas Ia and Ib, wherein R¹ is heterocycle optionally substituted with C₁₋₄ alkyl, NR⁸R⁹ or OR⁸, and R⁸ and R⁹ are independently H and methyl, or a pharmaceutically acceptable salt thereof. In one embodiment R¹ is heterocycle optionally substituted with CH₃, NH₂ or OH. In an embodiment R¹ is selected from piperidinyl, piperazinyl and morpholinyl, each of which may optionally be sustituted with CH₃.

Another embodiment of the invention relates to compounds of Formula I, including compounds of formulas Ia and Ib, wherein R¹ is NR⁶R⁷ and R⁶ and R⁷ are independently selected from H and C₁₋₄ alkyl that is optionally substituted with OH, OCH₃, heterocycle or cycloalkyl., or a pharmaceutically acceptable salt thereof.

Another embodiment of the invention relates to compounds of Formula I, including compounds of formulas Ia and Ib, wherein R¹ is as defined immediately above and R² is OR¹² and R¹² is C₁₋₄ alkyl optionally substituted with aryl or a pharmaceutically acceptable salt thereof. In one embodiment R¹² is methyl. In another embodiment R¹² is C₁₋₄ substituted with phenyl which optionally is substituted with Cl.

Another embodiment of the invention relates to compounds of Formula I, including compounds of formulas Ia and Ib, wherein R¹ is as defined above and R² is NR¹⁹R¹¹ and each of R¹⁰ and R¹¹ are independently selected from H, and C₁₋₆ alkyl that is optionally substituted with NH₂, OH, CONH₂, cycloalkyl, heterocycle, aryl, including phenyl that optionally substituted with Cl, or heteroaryl, including thiophenyl, or a pharmaceutically acceptable salt thereof. In an embodiment R¹⁰ and R¹¹ are independently selected from H and C₁₋₆ alkyl that is substituted with thiophene and NH₂.

Another embodiment of the invention relates to compounds of Formula I, including compounds of formulas Ia and Ib, wherein R¹ and R² are as defined immediately above and R³ is Cl, or a pharmaceutically acceptable salt thereof.

Another embodiment of the invention relates to compounds of Formula I, including compounds of formulas Ia and Ib, wherein R¹ and R² are as defined immediately above and R³ is CH₃, or a pharmaceutically acceptable salt thereof.

Another embodiment of the invention relates to compounds of Formula I, including compounds of formulas Ia and Ib, wherein R¹, R² and R³ are as defined immediately above and R⁴ is H, or a pharmaceutically acceptable salt thereof.

Another embodiment of the invention relates to compounds of Formula I, including compounds of formulas Ia and Ib, wherein R¹, R², R³ and R⁴ are as defined immediately above and R⁵ is H, or a pharmaceutically acceptable salt thereof.

Another embodiment of the invention relates to compounds of Formula I, including compounds of formulas Ia and Ib, wherein R⁶ and R⁷ are independently selected from H, C₁₋₄ alkyl that is optionally substituted with OH or OC₃ and N-ethyl, or a pharmaceutically acceptable salt thereof.

Another embodiment of the invention relates to compounds of Formula I, including compounds of formulas Ia and Ib, wherein R¹, R², R³, R⁴, R⁵, R⁶ and R⁷ are as defined immediately above and R⁸ and R⁹ are independently H or CH₃, or a pharmaceutically acceptable salt thereof.

Another embodiment of the invention relates to compounds of Formula I, including compounds of formulas Ia and Ib, wherein R¹, R², R³, R⁴, R⁵, R⁶, R⁷, R⁸ and R⁹ are as defined immediately above and R¹⁰ and R¹¹ are independently selected from H and C₁₋₆ alkyl that is optionally substituted with aryl, including specifically phenyl that optionally is substituted with C₁ or CH₃, or a pharmaceutically acceptable salt thereof.

Another embodiment of the invention relates to compounds of Formula I wherein R¹, R², R³, R⁴, R⁵, R⁶, R⁷, R⁸ and R⁹ are as defined immediately above and R¹⁰ and R¹¹ are independently selected from H and C₁₋₆ alkyl that is optionally substituted with heteroaryl, including specifically thiophenyl, or a pharmaceutically acceptable salt thereof.

Another embodiment of the invention relates to compounds of Formula I wherein R¹, R², R³, R⁴, R⁵, R⁶, R⁷, R⁸ and R⁹ are as defined immediately above and R¹⁰ and R¹¹ are independently selected from H and C₁₋₄ alkyl that optionally is substituted with OH, OCH₃, NH₂ and CONH₂, or a pharmaceutically acceptable salt thereof.

Another embodiment of the invention relates to compounds of Formula I wherein R¹, R², R³, R⁴, R⁵, R⁶, R⁷, R⁸ and R⁹ are as defined immediately above and R¹⁰ and R¹¹ are independently selected from H and aryl, including specifically phenyl, that optionally may be substituted with Cl, or a pharmaceutically acceptable salt thereof.

Another embodiment of the invention relates to compounds of Formula I, including compounds of formulas Ia and Ib, wherein R¹, R², R³, R⁴, R⁵, R⁶, R⁷, R⁸, R⁹, and R¹⁰ and R¹¹ are as defined immediately above and R¹² is selected from C₁₋₄ alkyl, including specifically CH₃,. or a pharmaceutically acceptable salt thereof.

It is contemplated herein that salts of compounds of formula I such as hydrochloride or trifluoroacetic acid salts include salts with multiple conjugates such as mono HCl, di-HCl, etc.

Compounds according to the invention include:
3-[(7-Methoxy-2-oxo-1,2-dihydro-[1,6]naphthyridine-3-carbonyl)-amino]-4-methyl-benzoic acid methyl ester (Example 28);
4-Chloro-3-[(7-methoxy-2-oxo-1,2-dihydro-[1,6]naphthyridine-3-carbonyl)-amino]-benzoic acid methyl ester (Example 30);
7-Methoxy-2-oxo-1,2-dihydro-[1,6]naphthyridine-3-carboxylic acid (5-benzylcarbamoyl-2-chloro-phenyl)-amide (Example 32);
7-Methoxy-2-oxo-1,2-dihydro-[1,6]naphthyridine-3-carboxylic acid (5-benzylcarbamoyl-2-methyl-phenyl)-amide (Example 33);
7-Methoxy-2-oxo-1,2-dihydro-[1,6]naphthyridine-3-carboxylic acid [5-(2-chloro-benzylcarbamoyl)-2-methyl-phenyl]-amide (Example 34);
7-Methoxy-2-oxo-1,2-dihydro-[1,6]naphthyridine-3-carboxylic acid [5-(3-chloro-benzylcarbamoyl)-2-methyl-phenyl]-amide (Example 35);
7-Methoxy-2-oxo-1,2-dihydro-[1,6]naphthyridine-3-carboxylic acid [5-(4-chloro-benzylcarbamoyl)-2-methyl-phenyl]-amide (Example 36);
7-Methoxy-2-oxo-1,2-dihydro-[1,6]naphthyridine-3-carboxylic acid {2-methyl-5-[(thiophen-2-ylmethyl)-carbamoyl]-phenyl}-amide (Example 37);
7-Methoxy-2-oxo-1,2-dihydro-[1,6]naphthyridine-3-carboxylic acid {2-methyl-5-[(thiophen-3-ylmethyl)-carbamoyl]-phenyl}-amide (Example 38);
7-Methoxy-2-oxo-1,2-dihydro-[1,6]naphthyridine-3-carboxylic acid [2-chloro-5-(2-chloro-benzylcarbamoyl)-phenyl]-amide (Example 39);
7-Methoxy-2-oxo-1,2-dihydro-[1,6]naphthyridine-3-carboxylic acid [2-chloro-5-(3-chloro-benzylcarbamoyl)-phenyl]-amide (Example 40);
7-Methoxy-2-oxo-1,2-dihydro-[1,6]naphthyridine-3-carboxylic acid [2-chloro-5-(4-chloro-benzylcarbamoyl)-phenyl]-amide (Example 41);
7-Methoxy-2-oxo-1,2-dihydro-[1,6]naphthyridine-3-carboxylic acid {2-chloro-5-[(thiophen-2-ylmethyl)-carbamoyl]-phenyl}-amide (Example 42);
7-Methoxy-2-oxo-1,2-dihydro-[1,6]naphthyridine-3-carboxylic acid {2-chloro-5-[(thiophen-3-ylmethyl)-carbamoyl]-phenyl}-amide (Example 43);
7-Methoxy-2-oxo-1,2-dihydro-[1,6]naphthyridine-3-carboxylic acid [5-(2-amino-1-phenyl-ethylcarbamoyl)-2-chloro-phenyl]-amide; hydrochloride (Example 45);
3-{[7-(2,3-Dihydroxy-propylamino)-2-oxo-1,2-dihydro-[1,6]naphthyridine-3-carbonyl]-amino}-4-methyl-benzoic acid methyl ester; hydrochloride (Example 49);
7-Methoxy-2-oxo-1,2-dihydro-[1,6]naphthyridine-3-carboxylic acid {5-[2-amino-1-(3-chloro-phenyl)-ethylcarbamoyl]-2-chloro-phenyl}-amide; hydrochloride (Example 51);
7-Methoxy-2-oxo-1,2-dihydro-[1,6]naphthyridine-3-carboxylic acid [5-(3-amino-1-thiophen-3-yl-propylcarbamoyl)-2-chloro-phenyl]-amide (Example 53);
7-Methoxy-2-oxo-1,2-dihydro-[1,6]naphthyridine-3-carboxylic acid [5-(2-amino-1-phenyl-ethylcarbamoyl)-2-methyl-phenyl]-amide; hydrochloride (Example 55);
7-Methoxy-2-oxo-1,2-dihydro-[1,6]naphthyridine-3-carboxylic acid {5-[2-amino-1-(3-chloro-phenyl)-ethylcarbamoyl]-2-methyl-phenyl}-amide; hydrochloride (Example 57);
7-Methoxy-2-oxo-1,2-dihydro-[1,6]naphthyridine-3-carboxylic acid [5-(2-amino-1-thiophen-3-yl-ethylcarbamoyl)-2-methyl-phenyl]-amide; hydrochloride (Example 59);
7-Methoxy-2-oxo-1,2-dihydro-[1,6]naphthyridine-3-carboxylic acid [5-(3-amino-1-phenyl-propylcarbamoyl)-2-methyl-phenyl]-amide; hydrochloride (Example 61);
7-Methoxy-2-oxo-1,2-dihydro-[1,6]naphthyridine-3-carboxylic acid [5-(3-amino-1-thiophen-3-yl-propylcarbamoyl)-2-methyl-phenyl]-amide (Example 63);
7-Methoxy-2-oxo-1,2-dihydro-[1,6]naphthyridine-3-carboxylic acid {5-[3-amino-1-(3-chloro-phenyl)-propylcarbamoyl]-2-methyl-phenyl}-amide; hydrochloride (Example 65);
7-Methoxy-2-oxo-1,2-dihydro-[1,6]naphthyridine-3-carboxylic acid {5-[3-amino-1-(3-chloro-phenyl)-propylcarbamoyl]-2-chloro-phenyl}-amide; hydrochloride (Example 67);
7-Methoxy-2-oxo-1,2-dihydro-[1,6]naphthyridine-3-carboxylic acid [5-(3-amino-1-phenyl-propylcarbamoyl)-2-chloro-phenyl]-amide; hydrochloride (Example 69);
7-Methoxy-2-oxo-1,2-dihydro-[1,6]naphthyridine-3-carboxylic acid [5-(2-amino-1-thiophen-3-yl-ethylcarbamoyl)-2-chloro-phenyl]-amide; hydrochloride (Example 71);
4-Chloro-3-{[7-(4-methyl-piperazin-1-yl)-2-oxo-1,2-dihydro-[1,6]naphthyridine-3-carbonyl]-amino}-benzoic acid methyl ester (Example 73);
4-Methyl-3-{[7-(4-methyl-piperazin-1-yl)-2-oxo-1,2-dihydro-[1,6]naphthyridine-3-carbonyl]-amino}-benzoic acid methyl ester (Example 74);
4-Methyl-3-{[7-(4-methyl-piperazin-1-yl)-2-oxo-1,2-dihydro-[1,8]naphthyridine-3-carbonyl]-amino}-benzoic acid methyl ester (Example 87);
4-Chloro-3-{[7-(4-methyl-piperazin-1-yl)-2-oxo-1,2-dihydro-[1,8]naphthyridine-3-carbonyl]-amino}-benzoic acid methyl ester (Example 88);
3-[(7-Methoxy-2-oxo-1,2-dihydro-[1,8]naphthyridine-3-carbonyl)-amino]-4-methyl-benzoic acid methyl ester (Example 89);
4-Chloro-3-[(7-methoxy-2-oxo-1,2-dihydro-[1,8]naphthyridine-3-carbonyl)-amino]-benzoic acid methyl ester (Example 90);
N-(5-(Benzylcarbamoyl)-2-chlorophenyl)-7-methoxy-2-oxo-1,2-dihydro-1,8-naphthyridine-3-carboxamide (Example 94);
N-(2-Chloro-5-(3-chlorobenzylcarbamoyl)phenyl)-7-methoxy-2-oxo-1,2-dihydro-1,8-naphthyridine-3-carboxamide (Example 95);
N-(5-(Benzylcarbamoyl)-2-methylphenyl)-7-methoxy-2-oxo-1,2-dihydro-1,8-naphthyridine-3-carboxamide (Example 98);
N-(5-(3-Chlorobenzylcarbamoyl)-2-methylphenyl)-7-methoxy-2-oxo-1,2-dihydro-1,8-naphthyridine-3-carboxamide (Example 99);
N-(5-(3-Amino-1-phenylpropylcarbamoyl)-2-methylphenyl)-7-methoxy-2-oxo-1,2-dihydro-1,8-naphthyridine-3-carboxamide (Example 100);
N-(5-(3-Amino-1-phenylpropylcarbamoyl)-2-chlorophenyl)-7-methoxy-2-oxo-1,2-dihydro-1,8-naphthyridine-3-carboxamide (Example 101);
   and the pharmaceutically acceptable salts of the foregoing compounds.

The compounds of formula I, as well as their salts, that have at least one asymmetric carbon atom may be present as racemic mixtures or different stereoisomers. The various isomers can be isolated by known separation methods, e.g., chromatography.

Compounds disclosed herein and covered by formula I above may exhibit tautomerism or structural isomerism. It is intended that the invention encompasses any tautomeric or structural isomeric form of these compounds, or mixtures of such forms, and is not limited to any one tautomeric or structural isomeric form depicted in the formulas above.

### Dosages

The compounds of the present invention that are inhibitors of DYRK1B are useful in the treatment, amelioration or control of cell proliferative disorders, in particular chemoprevention of cancer. Chemoprevention is defined as inhibiting the development of invasive cancer by either blocking the initiating mutagenic event or by blocking the progression of pre-malignant cells that have already suffered an insult of inhibiting tumor relapse. These compounds and formulations containing said compounds are anticipated to be particularly useful in the treatment or control of solid tumors, such as, for example, lung, pancreas, colon, breast, bone and prostate tumors.

Compounds that are inhibitors of DYRK1A are useful in the treatment, amelioration or control of Down syndrome and Alzheimer's disease.

A “therapeutically effective amount” or “effective amount” of a compound in accordance with this invention means an amount of compound that is effective to alleviate, ameliorate or control symptoms of disease or prolong the survival of the subject being treated.

The therapeutically effective amount or dosage of a compound according to this invention can vary within wide limits. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration; it may be given as continuous infusion.

### Compositions/Formulations

In an alternative embodiment, the present invention includes pharmaceutical compositions comprising at least one compound of formula I, or a pharmaceutically acceptable salt thereof, and a pharmaceutically acceptable excipient and/or carrier.

These pharmaceutical compositions can be suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a formula I compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

The pharmaceutical preparations of the invention can also contain preserving agents, solubilizing agents, stabilizing agents, wetting agents, emulsifying agents, sweetening agents, coloring agents, flavoring agents, salts for varying the osmotic pressure, buffers, coating agents or antioxidants. They can also contain other therapeutically valuable substances, including additional active ingredients other than those of formula I.

### General Synthesis of the Compounds According to the Invention

The present invention also provides methods for the synthesis of the 1,6- and 1,8-naphthyridines of the invention.

The compounds of the invention can be prepared by processes known in the art. Suitable processes for synthesizing these compounds are also provided in the examples. Generally, compounds of formula I can be synthesized according to one of the below described synthetic routes.

The key transformations are coupling reactions of carbonyl and carboxy starting materials and intermediates.

The starting materials are either commercially available or can be synthesized by methods known to those of ordinary skill in the art. In general, the compound of the invention can be synthesized according to the reactions shown is Schemes 13 and 14 below. The preparation of varius reactants useful in Schemes 13 and 14 is shown in Schemes 1-12. The amino-aldehydes needed for the synthesis of the 1,6-naphthyridines of this invention can be prepared by well known methods, for example, like the steps outlined in Schemes 1-7. The amino-aldehydes needed for the synthesis of the 1,8-naphthyridines can be prepared, for example, by the methods outlined in Schemes 8-9. These amino-aldehydes can be converted to the corresponding naphthyrdine 3-carboxylic acid esters by reaction with suitable malonate derivative (for example via dialkyl malonate as in Scheme 10, or via malonic acid mono ester mono acid chloride as in Scheme 11).

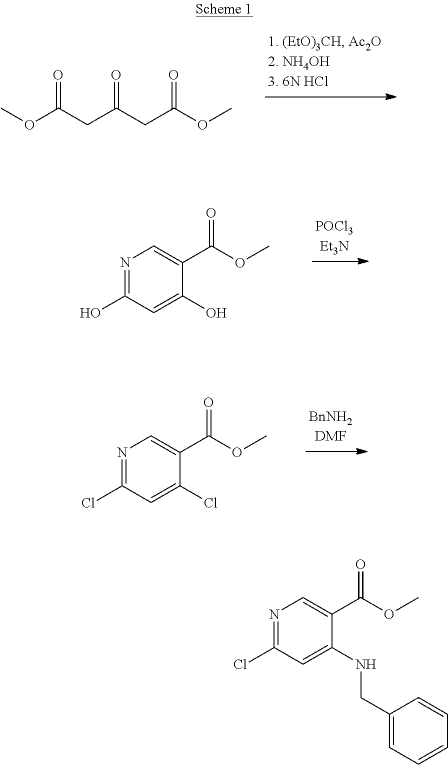

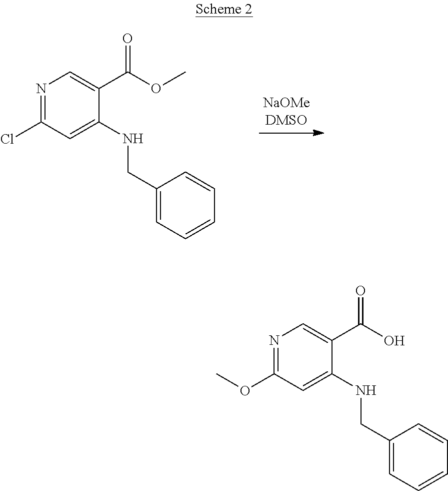

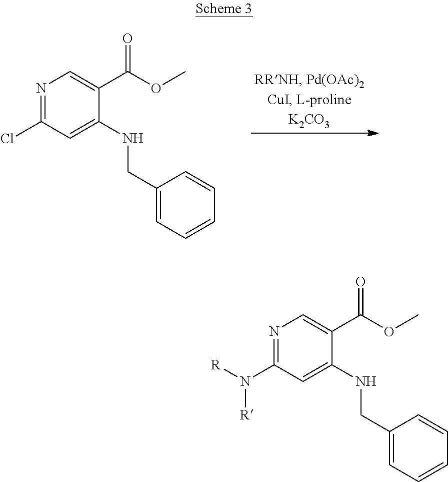

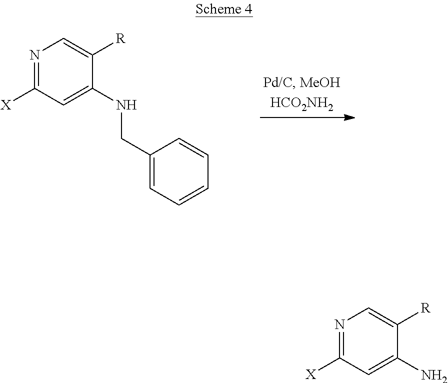

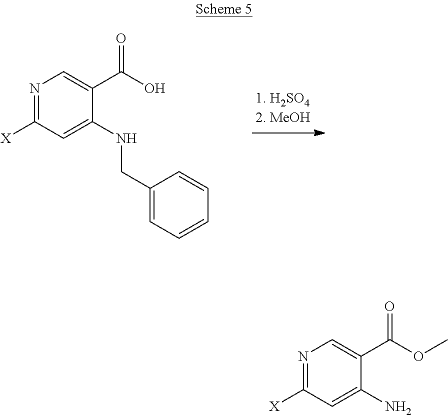

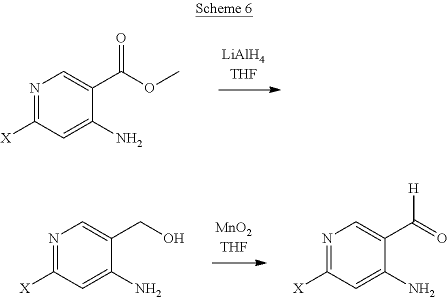

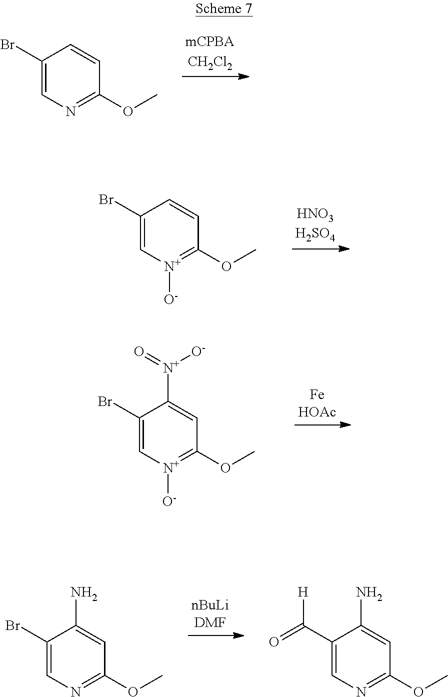

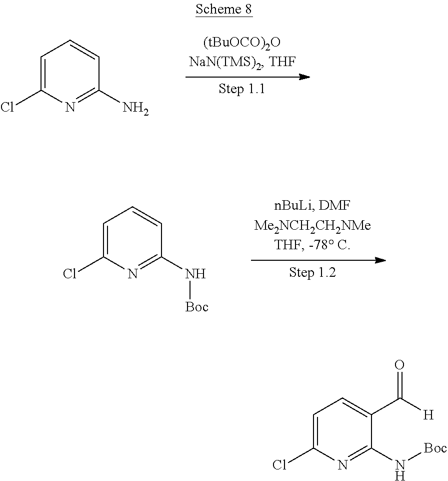

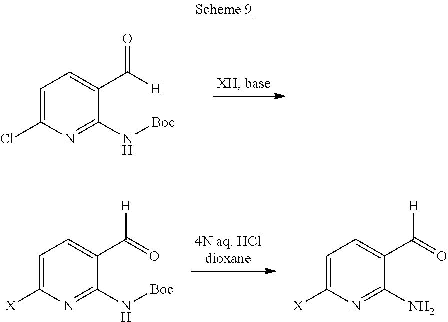

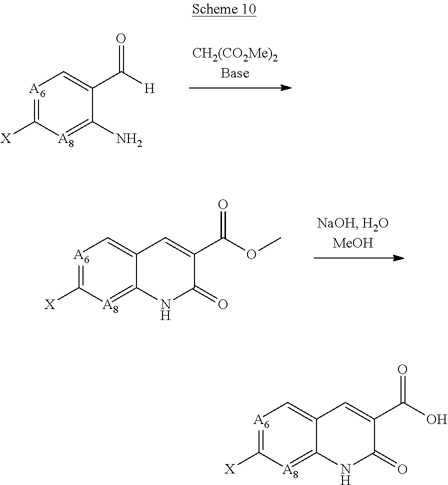

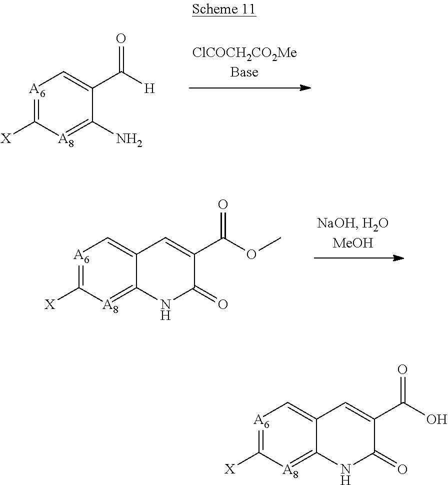

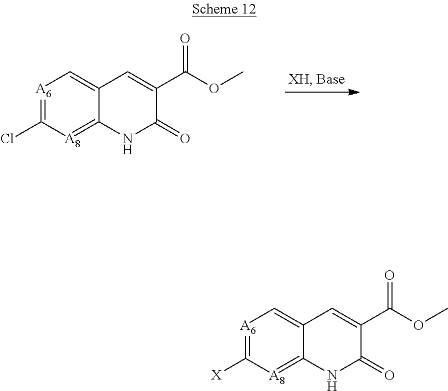

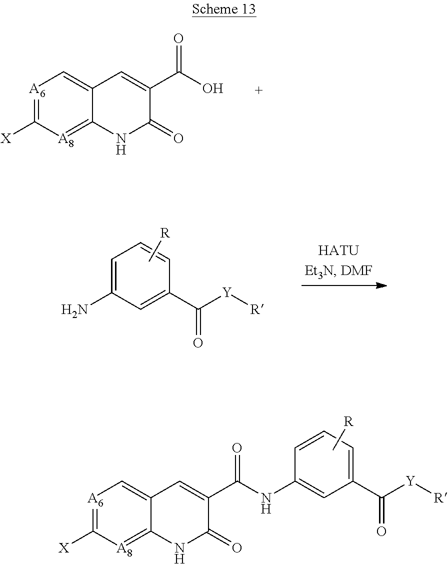

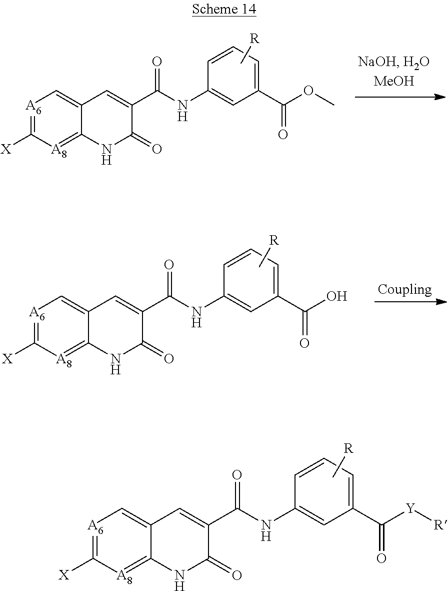

In case of certain amines that contain additional functional groups, appropriate protecting groups (for example tert-butoxy-carbonyl group) may be employed to facilitate synthesis. If such protecting groups are employed, the removal of such protecting groups to generate the compounds of the invention can be accomplished by standard methods known to those skilled in the art of organic synthesis.

### Crystal Forms

When the compounds of the invention are solids, it is understood by those skilled in the art that these compounds, and their salts, may exist in different crystal or polymorphic forms, all of which are intended to be within the scope of the present invention and specified formulas.

### EXAMPLES

The compounds of the present invention may be synthesized according to known techniques. The following examples and references are provided to aid the understanding of the present invention. The examples are not intended, however, to limit the invention, the true scope of which is set forth in the appended claims. The names of the final products in the examples were generated using AutoNom 2000 Add-in v4.0 SP2, (function in ISIS Draw, Elsevier/MDL), or AutoNom 2000 TT v4.01.305 (Elsevier/MDL), or functions available in ChemDraw Pro Control 11.0.2 (CambridgeSoft Corp.).

Abbreviations Used in the Examples:
Ac₂O acetic anhydride
Boc₂O di-tert-butyl dicarbonate
BuOH butanol
CDCl₃ chloroform-d
CH₂Cl₂ dichloromethane
CH₃CN acetonitrile
CH₂(CO₂CH₃)₂ dimethyl malonate
DCM dichloromethane
DEAD diethyl azodicarboxylate
DIPEA N,N-diisopropylethylamine
DMF N,N-dimethylformamide
DMSO dimethylsulfoxide
D₂O deuterium oxide
Et₃N triethylamine
EtOAc ethyl acetate
(EtO)₃CH triethyl orthoformate
EtOH ethanol
HATU O-(7-azabenzotriazol-1-yl)-N,N,N′,N′-tetramethyluronium hexafluorophosphate
HBTU O-(benzotriazol-1-yl)-N,N,N′,N′-tetramethyluronium hexafluorophosphate
HCl hydrogen chloride
HCO₂NH₄ ammonium formate
H₂O water
HOAc acetic acid
HPLC high performance liquid chromatography
H₂SO₄ sulfuric acid
LAH lithium aluminum hydride
LiAlH₄ lithium aluminum hydride
LiOH lithium hydroxide
K₂CO₃ potassium carbonate
K₃PO₄ potassium phosphate
MeCN acetonitrile
MeOH methanol
MgSO₄ magnesium sulfate
MnO₂ manganese dioxide
NaHCO₃ sodium bicarbonate
NaOH sodium hydroxide
Na₂SO₄ sodium sulfate
NH₃ ammonia
Pd(OAc)₂ palladium(II) acetate
POCl₃ phosphorous oxychloride
PPh₃ triphenylphosphine
TEA triethylamine
TFA trifluoroacetic acid
THF tetrahydrofuran
TLC thin layer chromatography

The following starting materials were purchased from the sources listed below:

2-Methyloxy-5-bromo pyridine Beijing Ouhe Technology Co., Ltd

3-Chloroperoxybenzic acid/Mcpba Beijing Ouhe Technology Co., Ltd

Piperidine Sinopharm Chemical Reagent Beijing Co., Ltd

Dimethyl malonate Beijing Ouhe Technology Co., Ltd

HATU Beijing Ouhe Technology Co., Ltd

n-Butyl lithium Sigma-Aldrich (Shanghai) Trading Co., Ltd

Methyl 3-amino-4-chlorobenzoate Alfa Aesar China (Tianjin) Co., Ltd

Methyl 3-aminobenzoate Alfa Aesar China (Tianjin) Co., Ltd

Methyl 3-amino-4-methylbenzoate Beijing Ouhe Technology Co., Ltd

Methyl 5-amino-2-methylbenzoate Beijing Ouhe Technology Co., Ltd

Methyl 5-amino-2-chlorobenzoate Beijing Ouhe Technology Co., Ltd

Dimethyl 1,3-acetonedicarboxylate Alfa Aesar China (Tianjin) Co., Ltd.

Triethyl orthoformate Beijing Ouhe Technology Co., Ltd

Phosphorus oxychloride Sinopharm Chemical Reagent Beijing Co., Ltd

Benzylamine Beijing Ouhe Technology Co., Ltd

1-Methylpiperazine Beijing Ouhe Technology Co., Ltd

Palladium(II) acetate Xian Kaili Technology Co., Ltd

Copper(I) iodide Alfa Aesar China (Tianjin) Co., Ltd.

L-Prolinamide Alfa Aesar China (Tianjin) Co., Ltd.

Sodium methoxide Sigma-Aldrich (Shanghai)

Lithium aluminum hydride Beijing Ouhe Technology Co., Ltd

Manganese(IV) oxide Beijing Ouhe Technology Co., Ltd

2-Amino-6-chloropyridine Oakwood

1-Methylpiperazine Aldrich

(2,2-Dimethyl-[1,3]-dioxolan-4-yl)-methylamine Aldrich

Dimethyl malonate Aldrich

HATU Chem-Impex

Methyl 3-aminobenzoate Alfa

Methyl 3-amino-4-methylbenzoate Alfa

Methyl 3-amino-4-chlorobenzoate TCI-US

Methyl 5-amino-2-methylbenzoate AKSCI

Methyl-5-amino-2-chlorobenzoate Oakwood

Sodium hexamethyldisilazane Alfa

### Example 1

### (2-Amino-2-phenyl-ethyl)-carbamic acid tert-butyl ester

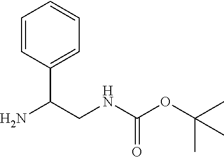

(2-Amino-2-phenyl-ethyl)-carbamic acid tert-butyl ester was prepared according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### (2-Hydroxy-2-phenyl-ethyl)-carbamic acid tert-butyl ester

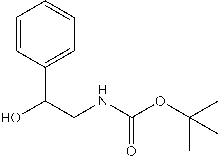

To a stirred solution of 2-amino-1-phenylethanol (20 g, 145.8 mmol) in THF (300 mL) was added the solution of Boc₂O (31.1 g, 153.1 mmol) in THF (100 mL) at 0° C. After addition, the mixture was stirred at room temperature for 0.5 hour. This mixture was concentrated to give the pure (2-hydroxy-2-phenyl-ethyl)-carbamic acid tert-butyl ester as a white solid. (Yield 34.4 g, 100%).

### Step B

### [2-(1,3-Dioxo-1,3-dihydro-isoindol-2-yl)-2-phenyl-ethyl]-carbamic acid tert-butyl ester

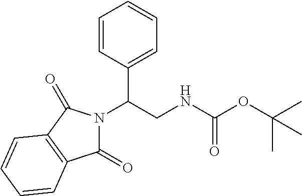

To a solution of (2-hydroxy-2-phenyl-ethyl)-carbamic acid tert-butyl ester (34.4 g, 145.0 mmol), phthalimide (21.3 g, 145 mmol), and PPh₃ (49.4 g, 188.5 mmol) was added drop-wise DEAD (32.8 g, 188.5 mmol) under stirring at 0° C. After addition, the mixture was stirred at room temperature for an additional 1 hour. The mixture was concentrated under reduced pressure. The residue was purified by column chromatography on silica gel (petroleum ether:ethyl acetate, 20:1 to 5:1) to give [2-(1,3-dioxo-1,3-dihydro-isoindol-2-yl)-2-phenyl-ethyl]-carbamic acid tert-butyl ester as a white solid. (Yield 39 g, 74%).

¹H NMR (300 MHz, CDCl₃): δ 7.88-7.80 (m, 2H), 7.74-7.68 (m, 2H), 7.49-7.47 (m, 2H), 7.38-7.26 (m, 3H), 5.56-5.50 (m, 1H), 4.83 (brs, 1H), 4.28-4.22 (m, 1H), 3.93-3.87 (m, 1H), 1.35 (s, 9H). LC-MS: [M-Boc+H]⁺ 267.

### Step C

### (2-Amino-2-phenyl-ethyl)-carbamic acid tert-butyl ester

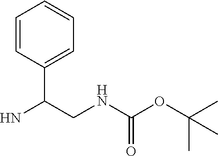

To a solution of [2-(1,3-dioxo-1,3-dihydro-isoindol-2-yl)-2-phenyl-ethyl]-carbamic acid tert-butyl ester (23 g, 63 mmol) in THF (180 mL) and MeOH (180 mL) was added 85% hydrazine hydrate (37 mL, 630 mmol) slowly. The resulting mixture was heated to 65° C. for 15 hours. The reaction mixture was cooled to room temperature, then concentrated to dryness. The residue was purified by column chromatography on silica gel (dichloromethane:MeOH, 100:1, 1% NH₃H₂O) to give (2-amino-2-phenyl-ethyl)-carbamic acid tert-butyl ester as a white solid. (Yield 7.4 g, 50%).

¹H NMR (300 MHz, CDCl₃): δ 7.35-7.24 (m, 5H), 4.81 (brs, 1H), 4.08-4.03 (m, 1H), 3.38-3.21 (m, 2H), 1.44 (s, 9H). LC-MS: [M+H]⁺ 237.

### Example 2

### (3-Amino-3-phenyl-propyl)-carbamic acid tert-butyl ester

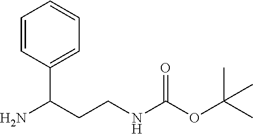

(3-Amino-3-phenyl-propyl)-carbamic acid tert-butyl ester was prepared according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### 3-Amino-1-phenyl-propan-1-ol

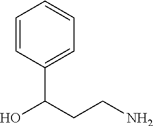

To a stirred suspension of LAH (20 g, 517 mmol) in dry THF (500 mL) was added a solution of 3-oxo-3-phenylpropanenitrile (30 g, 207 mmol) in dry THF (300 mL) drop-wise at 0° C. under nitrogen atmosphere. The mixture was warmed to 25° C. and then heated at 70° C. for 2 hours. After cooling to 0° C., a saturated solution of sodium hydroxide was added drop-wise and extracted with dichloromethane (200 mL). The organic solution was dried over anhydrous sodium sulfate and concentrated to dryness. The residue was purified by column chromatography (methanol:dichloromethane, 1:10) to afford 3-amino-1-phenyl-propan-1-ol. (Yield 30 g, crude).

LC-MS: [M+H]⁺ 152.

### Step B

### (3-Hydroxy-3-phenyl-propyl)-carbamic acid tert-butyl ester

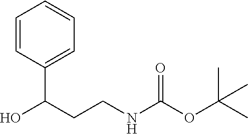

Et₃N (1.36 g, 14 mmol) was added to a solution of 3-amino-1-phenyl-propan-1-ol (1.7 g, 11.3 mmol) in THF (20 mL) under stirring. Boc₂O (3.0 g, 13.7 mmol) in THF (20 mL) was added dropwise to the solution at 0° C. Then the resulting mixture was warmed to room temperature and stirred for an additional 2 hours. The mixture was concentrated in vacuo. The residue was purified by column chromatography on silica gel (petroleum ether:ethyl acetate, 3:1) to give (3-hydroxy-3-phenyl-propyl)-carbamic acid tert-butyl ester. (Yield 1.7 g, 60%).

LC-MS: [M+Na]⁺ 274.

### Step C

### [3-(1,3-Dioxo-1,3-dihydro-isoindol-2-yl)-3-phenyl-propyl]-carbamic acid tert-butyl ester

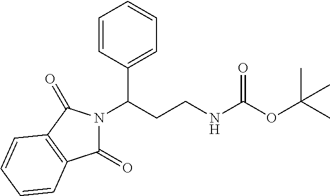

To a solution of (3-hydroxy-3-phenyl-propyl)-carbamic acid tert-butyl ester (10.4 g, 41.4 mmol), phthalimide (5.2 g, 36.6 mmol), and PPh₃ (14.6 g, 55.5 mmol) in THF (204 mL) was added dropwise DEAD (8.9 mL, 55 mmol) with stirring at 0° C. Then the resulting mixture was warmed to room temperature for an additional 2 hours. The mixture was concentrated in vacuo. The residue was purified by column chromatography on silica gel (petroleum ether:ethyl acetate, 3:1) to give [3-(1,3-dioxo-1,3-dihydro-isoindol-2-yl)-3-phenyl-propyl]-carbamic acid tert-butyl ester. (Yield 10.5 g, 66.8%).

¹H NMR (300 MHz, CDCl₃): δ 7.81-7.75 (m, 2H), 7.69-7.64 (m, 2H), 7.53-7.50 (m, 2H), 7.34-7.23 (m, 3H), 5.44-5.38 (m, 1H), 4.74 (brs, 1H), 3.29-3.07 (m, 2H), 2.83-2.75 (m, 1H), 2.51-2.42 (m, 1H), 1.42 (s, 9H). LC-MS: [M-Boc+H]⁺ , 281.

### Step D

### (3-Amino-3-phenyl-propyl)-carbamic acid tert-butyl ester

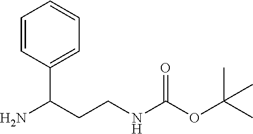

85% Hydrazine hydrate (5.1 mL, 74 mmol) was added to a solution of [3-(1,3-dioxo-1,3-dihydro-isoindol-2-yl)-3-phenyl-propyl]-carbamic acid tert-butyl ester (2.8 g, 7.4 mmol) in THF (25 mL) and MeOH (25 mL). The resulting mixture was heated to 65° C. for 6 hours. Then the precipitate was filtered, and the filtrate was concentrated in vacuo to give crude product which was purified by column chromatography on silica gel (dichloromethane:MeOH, 100:1, 1% NH₃H₂O) to give (3-amino-3-phenyl-propyl)-carbamic acid tert-butyl ester as an off-white solid. (Yield 1.7 g, 92%).

¹H NMR (300 MHz, CDCl₃): δ 7.31-7.18 (m, 5H), 6.82 (brs, 1H), 3.78-3.74 (m, 1H), 2.92 (brs, 2H), 1.82 (s, 2H), 1.63-1.61 (m, 2H), 1.37 (s, 9H). LC-MS: [M+H]⁺ 251.

### Example 3

### [2-Amino-2-(3-chloro-phenyl)-ethyl]carbamic acid tert-butyl ester

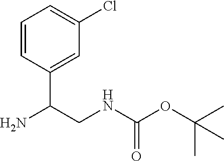

[2-Amino-2-(3-chloro-phenyl)-ethyl]-carbamic acid tert-butyl ester was prepared in an analogous process according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### (3-Chloro-phenyl)-hydroxy-acetonitrile

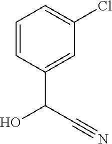

To a stirred suspension of KCN (5.04 g, 78 mmol) in methanol (20 mL) was added 3-chlorobenzaldehyde (7.0 g, 50 mmol) at 0° C. under nitrogen atmosphere. Then acetic acid (4.4 mL) was added dropwise at 0° C. After 30 minutes, the mixture was warmed to 15° C. and stirred for 5 hours. Then the reaction mixture was concentrated to dryness and extracted with ethyl acetate (200 mL). The organic solution was washed with water (3×25 mL), brine (25 mL), dried over anhydrous sodium sulfate and concentrated to dryness. The resulting residue was purified by column chromatography (ethyl acetate:petroleum ether, 1:15) to afford (3-chloro-phenyl)-hydroxy-acetonitrile. (Yield 8.2 g, 97%).

LC-MS: [M+Na]⁺ 190.

### Step B

### 2-Amino-1-(3-chloro-phenyl)-ethanol

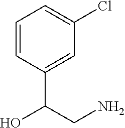

To a stirred suspension of LAH (2.36 g, 59 mmol) in dry THF (70 mL) was added a solution of (3-chloro-phenyl)-hydroxy-acetonitrile (4.0 g, 24 mmol) in dry THF (55 mL) dropwise at 0° C. under nitrogen atmosphere. The mixture was warmed to 25° C. and then heated at 60° C. for 2 hours. After cooling to 0° C., a saturated solution of sodium hydroxide was added dropwise and extracted with dichloromethane (200 mL). The organic solution was dried over anhydrous sodium sulfate and concentrated to dryness. The residue was purified by column chromatography (methanol:dichloromethane, 1:10) to afford 2-amino-1-(3-chloro-phenyl)-ethanol. (Yield 2.86 g, 70%).

LC-MS: [M+H]⁺ 172.

### Step C

### [2-(3-Chloro-phenyl)-2-hydroxy-ethyl]carbamic acid tert-butyl ester

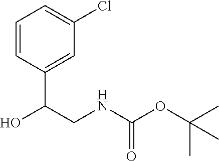

To a stirred solution of 2-amino-1-(3-chloro-phenyl)-ethanol (2.86 g, 16.7 mmol) in THF (100 mL) was added Boc₂O (4.3 g, 20 mmol). After 1 hour, the mixture was concentrated to dryness. The residue was purified by column chromatography (methanol:dichloromethane, 1:100) to afford [2-(3-chloro-phenyl)-2-hydroxy-ethyl]-carbamic acid tert-butyl ester. (Yield 3.9 g, 72%).

LC-MS: [M+Na]⁺ 294.

### Step D

### [2-(3-Chloro-phenyl)-2-(1,3-dioxo-1,3-dihydro-isoindol-2-yl)-ethyl]-carbamic acid tert-butyl ester

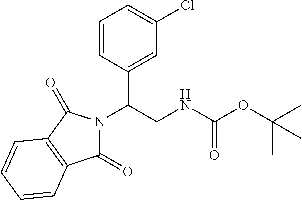

To a stirred solution of [2-(3-chloro-phenyl)-2-hydroxy-ethyl]-carbamic acid tert-butyl ester (20 g, 73.5 mmol), phthalimide (11.1 g, 73.5 mmol) and PPh₃ (25.1 g, 95.5 mmol) in THF (500 mL) was added DEAD (11.4 mL, 95.5 mmol) dropwise at −5 to 0° C. The reaction mixture was stirred at room temperature for 3 hours. Then the mixture was concentrated to dryness. The residue was purified by column chromatography (ethyl acetate:petroleum ether, 1:10) to afford [2-(3-chloro-phenyl)-2-(1,3-dioxo-1,3-dihydro-isoindol-2-yl)-ethyl]-carbamic acid tert-butyl ester. (Yield 20 g, 69%).

LC-MS: [M+H]⁺ 401.

### Step E

### [2-Amino-2-(3-chloro-phenyl)-ethyl]carbamic acid tert-butyl ester

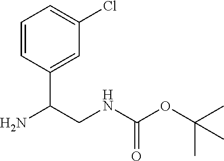

To a stirred solution of [2-(3-chloro-phenyl)-2-(1,3-dioxo-1,3-dihydro-isoindol-2-yl)-ethyl]-carbamic acid tert-butyl ester (2.5 g, 62 mmol) in THF (10 mL) and methanol (10 mL) was added hydrazine hydrate (3.1 g, 62 mmol). The mixture was heated to 55° C. for 1 hour. Then it was concentrated to dryness, dissolved in H₂O (5 mL) and extracted with ethyl acetate (50 mL). The organic mixture was concentrated and purified by column chromatography (methanol:dichloromethane, 1:100) to afford [2-amino-2-(3-chloro-phenyl)-ethyl]-carbamic acid tert-butyl ester. (Yield 1.325 g, 79%).

LC-MS: [M+H]⁺ 271.

### Example 4

### (3-Amino-3-thiophen-3-yl-propyl)-carbamic acid tert-butyl ester

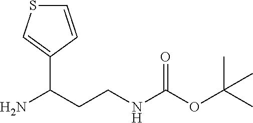

(3-Amino-3-thiophen-3-yl-propyl)-carbamic acid tert-butyl ester was prepared in an analogous process according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### 3-Amino-1-thiophen-3-yl-propan-1-ol

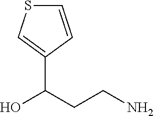

To a stirred suspension of LAH (1.45 g, 38.1 mmol) in dry THF (120 mL) was added a solution of 3-oxo-3-(thiophen-3-yl)propanenitrile (4.8 g, 31.8 mmol) in dry THF (40 mL) dropwise at 0° C. under nitrogen atmosphere. The mixture was warmed to 25° C. and then heated at 65° C. for 6 hours. After cooling to 0° C., a saturated solution of sodium hydroxide (2 mL) was added dropwise and the mixture was filtered. The filtrate was concentrated to dryness to give crude 3-amino-1-thiophen-3-yl-propan-1-ol which was used in next step without further purification. ¹H NMR (300 MHz, CDCl₃): δ 7.29-7.26 (m, 2H), 7.05 (dd, 1H, J₁=4.8 Hz, J₂=1.2 Hz), 5.04 (dd, 1H, J₁=8.1 Hz, J₂=3.0 Hz), 3.10-3.05 (m, 2H), 1.82-1.77 (m, 2H).

### Step B

### (3-Hydroxy-3-thiophen-3-yl-propyl)-carbamic acid tert-butyl ester

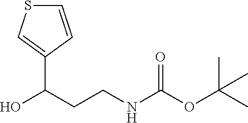

To a stirred solution of crude 3-amino-1-thiophen-3-yl-propan-1-ol (23 g) in THF (100 mL) was added Boc₂O (31.6 g, 146.3 mmol). The mixture was stirred at room temperature for 1 hour and then concentrated to dryness. The residue was purified by column chromatography (ethyl acetate:petroleum ether, 1:10) to afford (3-hydroxy-3-thiophen-3-yl-propyl)-carbamic acid tert-butyl ester. (Yield 21.5 g, 51% for two steps).

¹H NMR (300 MHz, CDCl₃): δ 8.08-8.06 (m, 1H), 7.55-7.53 (m, 1H), 7.34-7.30 (m, 1H), 5.10 (s, 1H), 3.52-3.48 (m, 2H), 3.13-3.09 (m, 2H), 1.42 (s, 9H). LC-MS: [M+Na]⁺ 280.

### Step C

### 3-(1,3-Dioxo-1,3-dihydro-isoindol-2-yl)-3-thiophen-3-yl-propyl]-carbamic acid tert-butyl ester

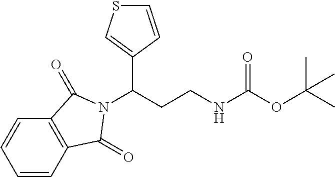

To a stirred solution of (3-hydroxy-3-thiophen-3-yl-propyl)-carbamic acid tert-butyl ester (21.5 g, 83.6 mmol), phthalimide (12.3 g, 83.6 mmol), and PPh₃ (28.5 g, 108.6 mmol) in THF (400 mL) was added DEAD (17.6 mL, 108.6 mmol) dropwise at 25° C. The mixture was stirred at room temperature for 14 hours, then concentrated. The residue was purified by column chromatography (ethyl acetate:petroleum ether, 1:6) to afford 3-(1,3-dioxo-1,3-dihydro-isoindol-2-yl)-3-thiophen-3-yl-propyl]-carbamic acid tert-butyl ester. (Yield 12 g, 38%).

¹H NMR (300 MHz, CDCl₃): δ 7.82-7.77 (m, 2H), 7.72-7.68 (m, 2H), 7.36 (d, 1H, J=1.8 Hz), 7.26-7.18 (m, 2H), 5.50 (dd, 1H, J₁=9.6 Hz, J₂=6 Hz), 4.65 (brs, 1H), 3.24-3.07 (m, 2H), 2.72-2.67 (m, 1H), 2.47-2.40 (m, 1H), 1.40 (s, 9H). LC-MS: [M+H-Boc]⁺ 287.

### Step D

### (3-Amino-3-thiophen-3-yl-propyl)-carbamic acid tert-butyl ester

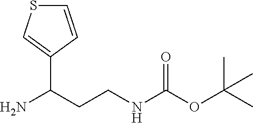

To a stirred solution of 3-(1,3-dioxo-1,3-dihydro-isoindol-2-yl)-3-thiophen-3-yl-propyl]-carbamic acid tert-butyl ester (12 g, 31.1 mmol) in methanol (150 mL) was added hydrazine hydrate (18 mL, 85% aqueous). The mixture was heated to reflux for 14 hours. After cooling to room temperature, the reaction mixture was filtered. The filtrate was concentrated and the residue was purified by column chromatography (methanol:dichloromethane, 1:50 to 1:20, 0.1% NH₃H₂O) to afford (3-amino-3-thiophen-3-yl-propyl)-carbamic acid tert-butyl ester. (Yield 7.6 g, 95%).

¹H NMR (300 MHz, CDCl₃): δ 7.49 (s, 1H), 7.25-7.08 (m, 2H), 6.82 (brs, 1H), 3.85 (t, 1H, J=6.0 Hz), 3.18-2.95 (m, 4H), 1.75-1.62 (m, 2H), 1.37 (s, 9H). LC-MS: [M+H]⁺ 257.

### Example 5

### (2-Amino-2-thiophen-3-yl-ethyl)-carbamic acid tert-butyl ester

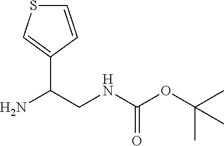

(2-Amino-2-thiophen-3-yl-ethyl)-carbamic acid tert-butyl ester was prepared in an analogous process according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### Hydroxy-thiophen-3-yl-acetonitrile

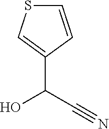

To a stirred suspension of KCN (18.6 g, 286 mmol) in methanol (100 mL) was added thiophene-3-carbaldehyde (20 mL, 178 mmol) at 0° C. under nitrogen atmosphere. Then acetic acid (4.4 mL) was added dropwise at 0° C. After 30 minutes, the mixture was warmed to 15° C. and stirred for 20 hours. NaHCO₃ (15 g) was added. The mixture was concentrated and extracted with ethyl acetate (200 mL). The organic mixture was washed with water (3×25 mL), brine (25 mL), dried over anhydrous sodium sulfate and concentrated to dryness. The residue was purified by column chromatography (ethyl acetate:petroleum ether, 1:10) to afford hydroxy-thiophen-3-yl-acetonitrile. (Yield 15 g, 60%).

LC-MS: [M+Na]⁺ 162.

### Step B

### 2-Amino-1-thiophen-3-yl-ethanol

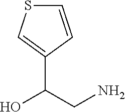

To a stirred suspension of LAH (8.7 g, 225 mmol) in dry THF (300 mL) was added a solution of hydroxy-thiophen-3-yl-acetonitrile (12.5 mL, 90 mmol) in dry THF (50 mL) dropwise at 0° C. under nitrogen atmosphere. Then the mixture was warmed to 25° C. and stirred overnight. After cooling to 10° C., H₂O (8.7 mL) was added to the solution, followed by NaOH solution (8.7 mL, 15%), then H₂O (26 mL). The reaction mixture was filtered and the filtration was concentrated to dryness to afford crude 2-amino-1-thiophen-3-yl-ethanol. (Yield 12.9 g, crude). LC-MS: [M+H]⁺ 144.

### Step C

### (2-Hydroxy-2-thiophen-3-yl-ethyl)-carbamic acid tert-butyl ester

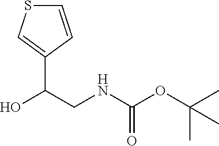

To a stirred solution of crude 2-amino-1-thiophen-3-yl-ethanol (12.9 g, crude) in THF (150 mL) was added Boc₂O (21.6 g, 99 mmol). After stirring for 1 hour, the mixture was concentrated to dryness which was purified by column chromatography (ethyl acetate:petroleum ether, 1:5) to afford (2-hydroxy-2-thiophen-3-yl-ethyl)-carbamic acid tert-butyl ester. (Yield 15.3 g, 70%).

LC-MS: [M+Na]⁺ 266.

### Step D

### [2-(1,3-Dioxo-1,3-dihydro-isoindol-2-yl)-2-thiophen-3-yl-ethyl]-carbamic acid tert-butyl ester

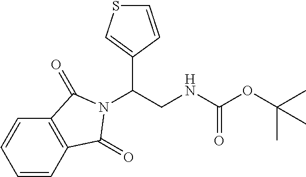

To a stirred solution of (2-hydroxy-2-thiophen-3-yl-ethyl)-carbamic acid tert-butyl ester (15.3 g, 63 mmol), pathalimide (9.5 g, 63 mmol), PPh₃ (21.4 g, 82 mmol) in THF (400 mL) was added DEAD (12.6 mL, 82 mmol) dropwise at 25° C. After 20 hours, the mixture was concentrated to dryness. The residue was purified by column chromatography (ethyl acetate:petroleum ether, 1:6) to afford [2-(1,3-dioxo-1,3-dihydro-isoindol-2-yl)-2-thiophen-3-yl-ethyl]-carbamic acid tert-butyl ester. (Yield 23 g, crude).

LC-MS: [M+Na]⁺ 395.

### Step E

### (2-Amino-2-thiophen-3-yl-ethyl)-carbamic acid tert-butyl ester

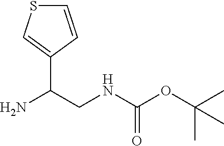

To a stirred solution of [2-(1,3-dioxo-1,3-dihydro-isoindol-2-yl)-2-thiophen-3-yl-ethyl]-carbamic acid tert-butyl ester (23 g, crude) in THF (100 mL) and methanol (100 mL) was added hydrazine hydrate (63 g, 1.26 mol). The mixture was heated to 60° C. for 2 hours and then cooled to 20° C. The reaction mixture was filtered and the filtration was concentrated to dryness. The residue was purified by column chromatography (methanol:dichloromethane, 1:50) to afford (2-amino-2-thiophen-3-yl-ethyl)-carbamic acid tert-butyl ester. (Yield 8.6 g, 57% for the two steps).

LC-MS: [M+H]⁺ 243.

### Example 6

### [3-Amino-3-(3-chloro-phenyl)-propyl]-carbamic acid tert-butyl ester

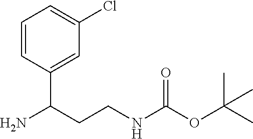

[3-Amino-3-(3-chloro-phenyl)-propyl]-carbamic acid tert-butyl ester was prepared in an analogous process according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### 3-Amino-1-(3-chloro-phenyl)-propan-1-ol

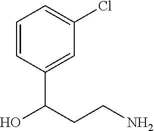

To a stirred suspension of LAH (16 g, 90 mmol) in dry THF (200 mL) was added a solution of 3-(3-chlorophenyl)-3-oxopropanenitrile (10.4 g, 270 mmol) in dry THF (200 mL) dropwise at 0° C. under nitrogen atmosphere. The mixture was warmed to 25° C. and then heated at 60° C. for 3 hours. After cooling to 0° C., a saturated solution of sodium hydroxide was added dropwise and extracted with ethyl acetate (200 mL). The solution was dried over anhydrous sodium sulfate and concentrated to dryness. The crude 3-amino-1-(3-chloro-phenyl)-propan-1-ol obtained was used in the next step without further purification. (Yield 14.5 g, crude).

LC-MS: [M+H]⁺ 186.

### Step B

### [3-(3-Chloro-phenyl)-3-hydroxy-propyl]carbamic acid tert-butyl ester

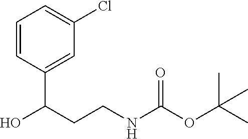

To a stirred solution of crude 3-amino-1-(3-chloro-phenyl)-propan-1-ol (29 g, 156 mmol) in THF (300 mL) was added Boc₂O (40.5 g, 187 mmol). After 0.5 hour, the mixture was concentrated to dryness. The residue was purified by column chromatography (ethyl acetate:petroleum ether, 1:20) to afford [3-(3-chloro-phenyl)-3-hydroxy-propyl]-carbamic acid tert-butyl ester. (Yield 23 g, 52%).

LC-MS: [M+Na]⁺ 308.

### Step C

### [3-(3-Chloro-phenyl)-3-(1,3-dioxo-1,3-dihydro-isoindol-2-yl)-propyl]-carbamic acid tert-butyl ester

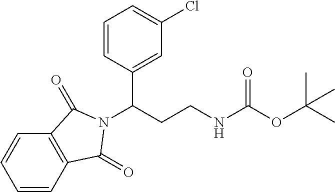

To a stirred solution of [3-(3-chloro-phenyl)-3-hydroxy-propyl]-carbamic acid tert-butyl ester (12 g, 42 mmol), phthalimide (6.2 g, 42 mmol), and PPh₃ (14.3 g, 55 mmol) in THF (150 mL) was added DEAD (9.0 mL, 55 mmol) dropwise at about 5° C. After 1 hour, the mixture was concentrated to dryness. The residue was purified by column chromatography (ethyl acetate:petroleum ether, 1:8) to afford [3-(3-chloro-phenyl)-3-(1,3-dioxo-1,3-dihydro-isoindol-2-yl)-propyl]-carbamic acid tert-butyl ester. (Yield 15.65 g, 90%).

LC-MS: [M+H]⁺ 415.

### Step D

### [3-Amino-3-(3-chloro-phenyl)-propyl]-carbamic acid tert-butyl ester

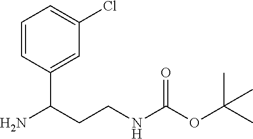

To a stirred solution of [3-(3-chloro-phenyl)-3-(1,3-dioxo-1,3-dihydro-isoindol-2-yl)-propyl]-carbamic acid tert-butyl ester (0.15 g, 0.36 mmol) in THF (2 mL) and methanol (2 mL) was added hydrazine hydrate (0.18 g, 3.6 mmol). The mixture was heated to 55° C. for 2 hours. Then the reaction mixture was concentrated and extracted with ethyl acetate (10 mL). The organic mixture was washed with water (3×1 mL), brine (1 mL), dried over anhydrous sodium sulfate and concentrated to dryness. The residue was purified by column chromatography (methanol:dichloromethane, 1:100) to afford [3-amino-3-(3-chloro-phenyl)-propyl]-carbamic acid tert-butyl ester. (Yield 0.061 g, 60%).

LC-MS: [M+H]⁺ 285.

### Example 7

### 4,6-Dihydroxy-nicotinic acid methyl ester

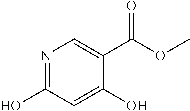

3-Oxo-pentanedioic acid dimethyl ester (100 g, 575 mmol), (EtO)₃CH (95 mL, 575 mmol), Ac₂O (108 mL) and HOAc (108 mL) were mixed and heated at 120° C. for 1.5 hours, then allowed to cool to 25° C. The volatiles were removed under reduced pressure while maintaining the water bath around 85° C. To the crude liquid was added aqueous NH₃ (25%, 150 mL) in portions with swirling in an ice bath and the stirring was continued for 1 hour. The mixture was acidified by the addition of 6 N HCl. The yellow precipitate was collected by filtration, boiled in toluene, cooled and filtered to give 4,6-dihydroxy-nicotinic acid methyl ester as an orange solid. (Yield 65 g, 67%).

¹H NMR (300 MHz, d₆-DMSO): δ 8.06 (s, 1H), 5.65 (s, 1H), 3.83 (s, 3H). LC-MS: [M+H]⁺ 170.

### Example 8

### 4,6-Dichloro-nicotinic acid methyl ester

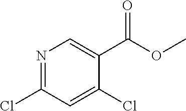

4,6-Dihydroxy-nicotinic acid methyl ester (3.9 g, 23 mmol) (from Example 7 supra) was added to POCl₃ (20 mL) at 0° C., then Et₃N (5 mL) was added to the mixture slowly. After addition, the mixture was heated at 120° C. for 2 hours, then allowed to cool to 0° C. The reaction solution was poured slowly and portion-wise into ice-water (100 mL). Solid K₂CO₃ was added to adjust pH to 8. The resulting precipitate was collected by filtration. The solid was dissolved in ethyl acetate (50 mL), dried over anhydrous Na₂SO₄, filtered and concentrated to afford 4,6-dichloro-nicotinic acid methyl ester. (Yield 4.6 g, 97.2%).

¹H NMR (301 MHz, CDCl₃) δ 8.85 (s, 1H), 7.47 (s, 1H), 3.97 (s, 3H). LC-MS: [M+H]⁺ 205.9.

### Example 9

### 4-Benzylamino-6-chloro-nicotinic acid methyl ester

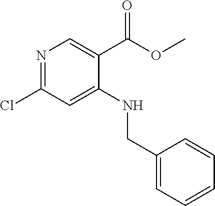

The mixture of 4,6-dichloro-nicotinic acid methyl ester (4.6 g, 22.3 mmol) (from Example 8 supra), benzylamine (2.63 g, 24.5 mmol) and Et₃N (6.75 g, 66.9 mmol) in DMF (10 mL) was stirred at room temperature for 3 hours. The reaction mixture was poured into water (250 mL) and extracted with ethyl acetate (3×200 mL). Combined organic layers was washed with water (100 mL), brine (100 mL), dried over anhydrous sodium sulfate, filtered and concentrated. The residue was purified by silica gel chromatography (eluting with petroleum ether/ethyl acetate, 20:1) to give 4-benzylamino-6-chloro-nicotinic acid methyl ester. (Yield 5.5 g, 89.1%).

¹H NMR (300 MHz, CDCl₃): δ 8.70 (s, 1H), 8.53 (s, 1H), 7.38-7.30 (m, 5H), 6.56 (s, 1H), 4.44 (d, 2H, J=5.6 Hz), 3.90 (s, 3H). LC-MS: [M+H]⁺ 277.

### Example 10

### 4-Benzylamino-6-methoxy-nicotinic acid

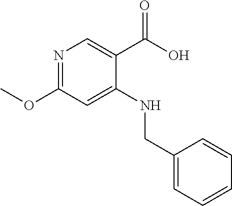

A mixture of 4-benzylamino-6-chloro-nicotinic acid methyl ester (10.5 g, 38.04 mmol) (from Example 9 supra) and sodium methoxide (20.5 g, 380.43 mmol) in DMSO (120 mL) was heated at 135° C. for 3 hours. TLC showed that compound 4-benzylamino-6-chloro-nicotinic acid methyl ester was consumed completely. It was cooled to room temperature, diluted with water (500 mL), extracted with dichloromethane (200 mL). The aqueous phase was acidified with 1 N HCl to pH 3-4. The precipitate was collected by filtration, washed with water, dried under reduced pressure to give 4-benzylamino-6-methoxy-nicotinic acid as a white powder. (Yield 8.3 g, 84%).

¹H NMR (300 MHz, d₆-DMSO) δ 8.48 (s, 2H), 7.30 (dt, 5H, J=6.4, 4.2 Hz), 5.86 (s, 1H), 4.46 (d, 2H, J=5.8 Hz), 3.77 (s, 3H). LC-MS: [M+H]⁺ 259.

### Example 11

### 4-Amino-6-methoxy-nicotinic acid methyl ester

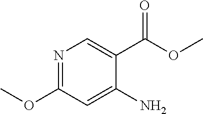

4-Benzylamino-6-methoxy-nicotinic acid (7.3 g, 28.29 mmol) (from Example 10 supra) was dissolved in conc. H₂SO₄ (37 mL) and the resulting solution was stirred at room temperature for 4 hours. Then it was added into MeOH (200 mL) at 0° C. and the solution was heated at reflux overnight. It was then cooled to 0° C., basified with solid NaHCO₃ and saturated aqueous NaHCO₃ (about 500 mL), extracted with dichloromethane (3×300 mL), washed with water (2×200 mL) and brine (200 mL), dried over anhydrous sodium sulfate, filtered and concentrated under reduced pressure to give 4-amino-6-methoxy-nicotinic acid methyl ester as a white powder. (Yield 4.24 g, 82.8%).

¹H NMR (300 MHz, CDCl₃) δ 8.63 (s, 1H), 6.03 (s, 2H), 5.88 (s, 1H), 3.92 (s, 3H), 3.87 (s, 3H).

LC-MS: [M+H]⁺ 183.

### Example 12

### (4-Amino-6-methoxy-pyridin-3-yl)-methanol

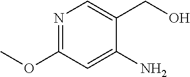

To a suspension of LiAlH₄ (2.57 g, 67.58 mmol) in THF (30 mL) was added a solution of 4-amino-6-methoxy-nicotinic acid methyl ester (4.92 g, 27.03 mmol) (from Example 11 supra) in THF (45 mL) at 10° C. under N₂. The resulting reaction mixture was warmed up to room temperature and stirred for 3 hours. TLC showed that little 4-amino-6-methoxy-nicotinic acid methyl ester remained in the reaction mixture. Then it was cooled to 0° C. and water (15 mL) was added dropwise. A lot of white solid was formed. The mixture was warmed to room temperature and stirred for 20 minutes. Then it was diluted with THF (100 mL), filtered and the filter cake was washed with THF. The filtrate was concentrated and co-evaporated with EtOH to give (4-amino-6-methoxy-pyridin-3-yl)-methanol as an off-white solid. (Yield 3.78 g, 91%). ¹H NMR (300 MHz, d₆-DMSO) δ 7.62 (s, 1H), 5.90 (s, 1H), 5.74 (s, 2H), 4.91 (t, 1H, J=5.4 Hz), 4.34 (d, 2H, J=5.4 Hz), 3.72 (s, 3H). LC-MS: [M+H]⁺ 155.

### Example 13

### 4-Amino-6-methoxy-pyridine-3-carbaldehyde

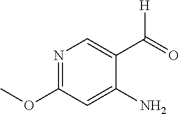

To a solution of (4-amino-6-methoxy-pyridin-3-yl)-methanol (3.78 g, 24.55 mmol) (from Example 12 supra) in THF (100 mL) was added MnO₂ (21.34 g, 245.5 mmol) at room temperature. The resulting reaction mixture was stirred overnight under N₂. TLC showed that little (4-amino-6-methoxy-pyridin-3-yl)-methanol remained in the reaction mixture. It was filtered and the filter cake was washed with THF. The filtrate was concentrated under reduced pressure to dryness to give 4-amino-6-methoxy-pyridine-3-carbaldehyde as a yellow solid. (Yield 3.73 g, 100%).

¹H NMR (300 MHz, d₆-DMSO) δ 9.76 (s, 1H), 8.31 (s, 1H), 7.43 (s, 2H), 5.95 (s, 1H), 3.83 (s, 3H). LC-MS: [M+H]⁺ 153.

### Example 14

### 7-Methoxy-2-oxo-1,2-dihydro-[1,6]naphthyridine-3-carboxylic acid methyl ester

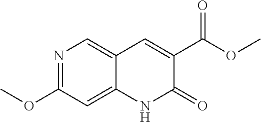

To a solution of 4-amino-6-methoxy-pyridine-3-carbaldehyde (3.668 g, 24.13 mmol) (from Example 13 supra) in MeOH (460 mL) was added CH₂(CO₂CH₃)₂ (8.82 g, 66.84 mmol), AcOH (145 mg, 2.41 mmol) and piperidine (5.74 g, 67.56 mmol) at room temperature. The resulting reaction mixture was heated to 60° C. for 18 hours under N₂. LC-MS showed that little 4-amino-6-methoxy-pyridine-3-carbaldehyde existed in the reaction mixture. Then it was cooled to room temperature and concentrated under reduced pressure. The residue was washed with ether (100 mL) and the precipitate was collected by filtration, dried under reduced pressure to give 7-methoxy-2-oxo-1,2-dihydro-[1,6]naphthyridine-3-carboxylic acid methyl ester as a white solid. (Yield 5.1 g, 90%).

¹H NMR (300 MHz, d₆-DMSO) δ 12.03 (s, 1H), 8.75 (s, 1H), 8.61 (s, 1H), 6.52 (s, 1H), 3.95 (s, 3H), 3.83 (s, 3H). LC-MS: [M+H]⁺ 235.

### Example 15

### 7-Methoxy-2-oxo-1,2-dihydro-[1,6]naphthyridine-3-carboxylic acid

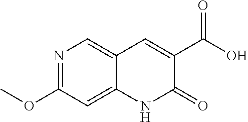

A mixture of 7-methoxy-2-oxo-1,2-dihydro-[1,6]naphthyridine-3-carboxylic acid methyl ester (5.1 g, 21.79 mmol) (from Example 14 supra), NaOH (2.62 g, 65.38 mmol) in MeOH (70 mL) and water (70 mL) was heated at 65° C. for 18 hours. A lot of white solid was formed during reaction. Then the reaction mixture was cooled to room temperature, diluted with water and concentrated under reduced pressure to remove most of the MeOH. The resulting mixture was acidified with 1 N aqueous HCl to ph 3-4. White precipitate formed was collected by filtration, washed with water and dried under reduced pressure to give 7-methoxy-2-oxo-1,2-dihydro-[1,6]naphthyridine-3-carboxylic acid as a white solid. (Yield 4.7 g, 98%).

¹H NMR (300 MHz, d₆-DMSO) δ 13.41 (s, 2H), 8.92 (s, 2H), 6.61 (s, 1H), 3.96 (s, 3H). LC-MS: [M+H]⁺ 221.

### Example 16

### 4-Benzylamino-6-(4-methyl-piperazin-1-yl)-nicotinic acid methyl ester

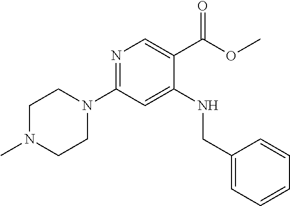

The mixture of 4-benzylamino-6-chloro-nicotinic acid methyl ester (14.46 g, 52.39. mmol) (from Example 9 supra), 1-methylpiperazine (9.43 g, 94.3 mmol), Pd(OAc)₂ (868 mg), CuI (868 mg), K₂CO₃ (18 g, 130.53 mmol), L-proline (868 mg) in 1-methylpyrrolidin-2-one (30 mL) was stirred at 100° C. for 36 hours. It was diluted with water and extracted with CH₂Cl₂ (3×50 mL). After the solvent was removed, the residue was purified by flash chromatography over silica gel (eluting with CH₂Cl₂/MeOH, 40/1 to 8/1) to give 4-benzylamino-6-(4-methyl-piperazin-1-yl)-nicotinic acid methyl ester (14.2 g, 80%). LC-MS: [M+H]⁺ 341.

### Example 17

### [4-Benzylamino-6-(4-methyl-piperazin-1-yl)-pyridin-3-yl]-methanol

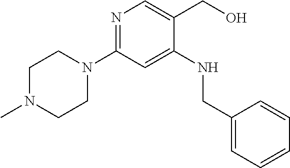

4-Benzylamino-6-(4-methyl-piperazin-1-yl)-nicotinic acid methyl ester (3.47 g, 10.2 mmol) (from Example 16 supra) was dissolved in THF (20 mL). LiAlH₄ (0.7 g, 18.36 mmol) was added to the mixture in small batches at 0° C. After addition, the mixture was stirred at room temperature under nitrogen overnight. At that time, Na₂SO₄.10H₂O (1.4 g) was added and the mixture was filtered and washed with THF (500 mL). The filtrate was concentrated to give [4-benzylamino-6-(4-methyl-piperazin-1-yl)-pyridin-3-yl]-methanol as white solid. (Yield 3.22 g, 100%).

¹H NMR (300 MHz, d₆-DMSO): δ 7.59 (s, 1H), 7.39-7.24 (m, 5H), 6.23 (t, 1H, J=5.4 Hz), 5.79 (s, 2H), 4.93 (t, 1H, J=5.4 Hz), 4.40-4.37 (m, 5H), 3.34-3.29 (m, 4H), 2.17 (s, 3H). LC-MS: [M+H]⁺ 313.

### Example 18

### [4-Amino-6-(4-methyl-piperazin-1-yl)-pyridin-3-yl]-methanol

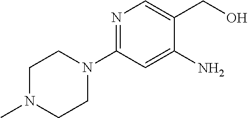

The mixture of [4-benzylamino-6-(4-methyl-piperazin-1-yl)-pyridin-3-yl]-methanol (3.22 g, 10.31 mmol) (from Example 17 supra), ammonium formate (3.25 g, 51.54 mmol), 10% Pd/C (3.22 g) in MeOH (30 mL) was heated at 80° C. for 16 hours. The mixture was filtered and the filtrate was concentrated and purified by silica gel chromatography, eluting with CH₂Cl₂/MeOH/NH₃.H₂O=300/5/1.5 to 300/20/1.5 V/V/V to give [4-amino-6-(4-methyl-piperazin-1-yl)-pyridin-3-yl]-methanol. (Yield 206 mg, 9%).

¹H NMR (300 MHz, d₆-DMSO): δ 7.59 (s, 1H), 5.93 (s, 1H), 5.50 (s, 2H), 4.28 (d, 2H, J=5.4 Hz), 3.32-3.29 (m, 2H), 2.36-2.33 (m, 4H), 2.19 (s, 3H). LC-MS: [M+H]⁺ 223.

### Example 19

### 4-Amino-6-(4-methyl-piperazin-1-yl)-pyridine-3-carbaldehyde

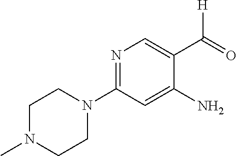

The mixture of [4-amino-6-(4-methyl-piperazin-1-yl)-pyridin-3-yl]-methanol (206 mg, 0.93 mmol) (from Example 18 supra), MnO₂ (807 mg, 9.28 mmol) in CH₂Cl₂ (20 mL) was stirred at room temperature overnight. Then the mixture was filtered. The filtrate was concentrated to give 4-amino-6-(4-methyl-piperazin-1-yl)-pyridine-3-carbaldehyde which was used in the next reaction without purification. (Yield 180 mg, 88%).

LC-MS: [M+H]⁺ 221.

### Example 20

### 7-(4-Methyl-piperazin-1-yl)-2-oxo-1,2-dihydro-[1,6]naphthyridine-3-carboxylic acid methyl ester

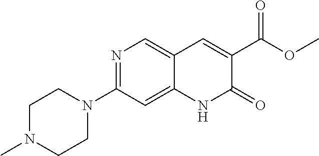

The mixture of 4-amino-6-(4-methyl-piperazin-1-yl)-pyridine-3-carbaldehyde (180 mg, 0.82 mmol) (from Example 19 supra), CH₂(COOMe)₂ (300 mg, 2.27 mmol), AcOH (5 mg, 0.082 mmol) and piperidine (195 mg, 2.29 mmol) in MeOH (20 mL) was heated at 60° C. for 18 hours. After cooling, the mixture was concentrated to give 7-(4-methyl-piperazin-1-yl)-2-oxo-1,2-dihydro-[1,6]naphthyridine-3-carboxylic acid methyl ester which was used without further purification. (Yield 247 mg, 100%).

¹H NMR (300 MHz, d₆-DMSO): δ 8.60 (s, 1H), 8.46 (s, 1H), 6.29 (s, 1H), 3.57 (t, 4H, J=4.2 Hz), 2.75 (brs, 3H), 2.40 (t, 4H, J=4.5 Hz), 2.22 (s, 3H,). LC-MS: [M+H]⁺ 302.9.

### Example 21

### 7-(4-Methyl-piperazin-1-yl)-2-oxo-1,2-dihydro-[1,6]naphthyridine-3-carboxylic acid

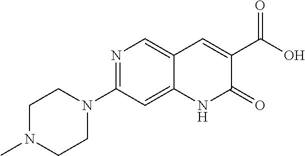

A mixture of 7-(4-methyl-piperazin-1-yl)-2-oxo-1,2-dihydro-[1,6]naphthyridine-3-carboxylic acid methyl ester (232 mg, 0.77 mmol) (from Example 20 supra), NaOH (92 mg, 2.31 mmol) in MeOH (3 mL) and water (3 mL) was heated at 65° C. for 18 hours. After cooling, mixture was diluted with water and concentrated under reduced pressure. Resulting solution was extracted with ethyl acetate (3×5 mL). And water layer was acidified with 2N aqueous HCl, precipitate formed was collected by filtration and washed with water to give 7-(4-methyl-piperazin-1-yl)-2-oxo-1,2-dihydro-[1,6]naphthyridine-3-carboxylic acid. (Yield 166 mg, 75%).

### Example 22

### 4-Amino-6-chloro-nicotinic acid methyl ester

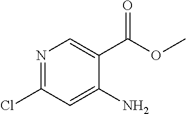

A solution of 4-benzylamino-6-chloro-nicotinic acid methyl ester (5.5 g, 19.8 mmol) (from Example 9 supra) in conc. H₂SO₄ (24 mL) was left standing for 15 minutes at 0° C. Then the dark solution was poured into ice-water (200 mL) while stirring. The solution was neutralized to pH4 using K₂CO₃. The precipitate was filtered off and the pH of the filtrate was adjusted to 10 with K₂CO₃, and the solution was extracted with ethyl acetate (300 mL). The extracts were combined and washed with water (100 mL), brine (50 mL) and dried over anhydrous sodium sulfate. The drying agent was removed by filtration and the solvent was evaporated to give 4-amino-6-chloro-nicotinic acid methyl ester which was used directly in the next step without further purification. (Yield 3.8 g, 103%, crude).

¹H NMR (300 MHz, CDCl₃): δ 8.69 (s, 1H), 6.57 (s, 1H), 3.90 (s, 3H), 1.62 (brs, 2H). LC-MS: [M+H]⁺ 186.9.

### Example 23

### (4-Amino-6-chloro-pyridin-3-yl)-methanol

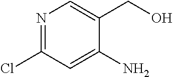

To a stirred suspension of LiAlH₄ (1.0 g, 26.3 mmol) in dry THF (500 mL) at 0° C. under nitrogen was added dropwise a solution of 4-amino-6-chloro-nicotinic acid methyl ester (2.6 g, 13.9 mmol) (from Example 22 supra) in THF (100 mL). After the addition, the reaction mixture was stirred at this temperature for 1 hour. The reaction was quenched slowly with water (1.5 mL) and filtered. The organic solution was evaporated to give (4-amino-6-chloro-pyridin-3-yl)-methanol as a white solid which was used directly in the next step. (Yield 2.2 g, 100%).

LC-MS: [M+H]⁺ 159.0.

### Example 24

### 4-Amino-6-chloro-pyridine-3-carbaldehyde

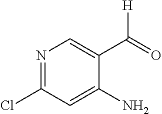

To a solution of (4-amino-6-chloro-pyridin-3-yl)-methanol (0.335 g, 2.1 mmol) (from Example 23 supra) in dichloromethane (100 mL) at room temperature was added MnO₂ (1.9 g, 21.8 mmol). The reaction mixture was stirred at this temperature for 16 hours then filtered. Solvent was evaporated to give 4-amino-6-chloro-pyridine-3-carbaldehyde as a white solid which was used directly in the next step. (Yield 0.275 g, 83.9%).

¹H NMR (300 MHz, CDCl₃): δ 9.90 (s, 1H), 8.38 (s, 1H), 6.58 (s, 1H), 1.57 (s, 2H). LC-MS: [M+H]⁺ 157.1.

### Example 25

### 7-Chloro-2-oxo-1,2-dihydro-[1,6]naphthyridine-3-carboxylic acid methyl ester

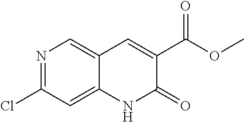

To a solution of 4-amino-6-chloro-pyridine-3-carbaldehyde (1.6 g, 10.2 mmol) (from Example 24 supra) in DMSO (10 mL) at room temperature, was added dimethyl malonate (6.4 g, 48.4 mmol) and DL-proline (1.5 g, 13 mmol) successively. The reaction mixture was stirred at room temperature for 16 hours and then at 65° C. for 2 hours. The mixture was poured into water (80 mL), and extracted with dichloromethane (4×100 mL). The organic phase was washed with water (3.×100 mL), brine (50 mL) and dried to give a crude product. It was purified by chromatography (silica gel, 200-300 mesh, eluting with a mixture of petroleum ether and ethyl acetate (3:2, v/v)) to give 7-chloro-2-oxo-1,2-dihydro-[1,6]naphthyridine-3-carboxylic acid methyl ester. (Yield 2.0 g, 82.3%).

¹H NMR (300 MHz, DMSO): δ 12.41 (s, 1H), 8.86 (s, 1H), 8.64 (s, 1H), 7.22 (s, 1H), 3.83 (s, 3H). LC-MS: [M+H]⁺ 239.0.

### Example 26

### 7-[(2,2-Dimethyl-[1,3]dioxolan-4-ylmethyl)-amino]-2-oxo-1,2-dihydro-[1,6]naphthyridine-3-carboxylic acid methyl ester

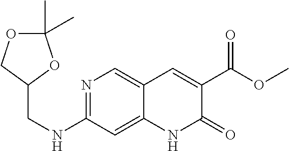

To a stirred solution of 7-chloro-2-oxo-1,2-dihydro-[1,6]naphthyridine-3-carboxylic acid methyl ester (0.515 g, 2.15 mmol) (from Example 25 supra) in DMSO (8 mL) was added triethylamine (0.633 g, 6.26 mmol) and C-(2,2-dimethyl-[1,3]dioxolan-4-yl)-methylamine (0.552 g, 4.2 mmol) at room temperature under nitrogen. Then the reaction mixture was stirred at 140° C. for two hours under nitrogen. The mixture was cooled down and poured into water (35 mL), then extracted with dichloromethane (3×100 mL). The organic phase was washed with water (3×80 mL), brine (80 mL) and dried to give 7-[(2,2-dimethyl-[1,3]dioxolan-4-ylmethyl)-amino]-2-oxo-1,2-dihydro-[1,6]naphthyridine-3-carboxylic acid methyl ester as a solid which was used directly in the next step without further purification. (Yield 0.61 g, 85.2%).

LC-MS: [M+H]⁺ 334.0.

### Example 27

### 7-[(2,2-Dimethyl-[1,3]dioxolan-4-ylmethyl)-amino]-2-oxo-1,2-dihydro-[1,6]naphthyridine-3-carboxylic acid

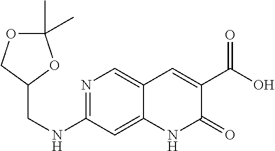

To a stirred solution of 7-[(2,2-dimethyl-[1,3]dioxolan-4-ylmethyl)-amino]-2-oxo-1,2-dihydro-[1,6]naphthyridine-3-carboxylic acid methyl ester (180 mg, 0.54 mmol) (from Example 26 supra) in methanol (40 mL) was added a solution of sodium hydroxide (50 mg, 12.5 mmol) in water (6 mL) at room temperature. After the addition, the solution was stirred at this temperature for 16 hours. The solution was evaporated at reduced pressure and the residue was diluted with water (20 mL), then extracted with dichloromethane (3×20 mL) to give 7-[(2,2-dimethyl-[1,3]dioxolan-4-ylmethyl)-amino]-2-oxo-1,2-dihydro-[1,6]naphthyridine-3-carboxylic acid as a solid which was used directly in the next step without further purification. (Yield 150 mg, 87.2%). LC-MS: [M+H]⁺ 320.0.

### Example 28

### 3-[(7-Methoxy-2-oxo-1,2-dihydro-[1,6]naphthyridine-3-carbonyl)-amino]-4-methyl-benzoic acid methyl ester

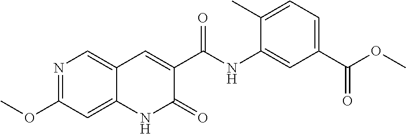

3-[(7-Methoxy-2-oxo-1,2-dihydro-[1,6]naphthyridine-3-carbonyl)-amino]-4-methyl-benzoic acid methyl ester was prepared from 7-methoxy-2-oxo-1,2-dihydro-[1,6]naphthyridine-3-carboxylic acid (from Example 15 supra) (2.4 g, 10.9 mmol) and 3-amino-4-methyl-benzoic acid methyl ester by following the method in Example 30. (Yield 3.2 g, 80%).

¹H NMR (300 MHz, d₆-DMSO) δ 12.12 (s, 1H), 8.97 (s, 2H), 8.89 (s, 1H), 7.64 (dd, 1H, J=7.8, 1.7 Hz), 7.41 (d, 1H, J=8.0 Hz), 6.61 (s, 1H), 3.95 (s, 3H), 3.86 (s, 3H), 2.42 (s, 3H). LC-MS: [M+H]⁺ 368.

### Example 29

### 3-[(7-Methoxy-2-oxo-1,2-dihydro-[1,6]naphthyridine-3-carbonyl)-amino]-4-methyl-benzoic acid

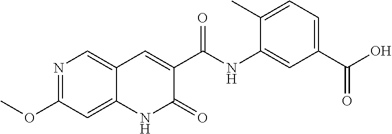

3-[(7-Methoxy-2-oxo-1,2-dihydro-[1,6]naphthyridine-3-carbonyl)-amino]-4-methyl-benzoic acid was prepared from 3-[(7-methoxy-2-oxo-1,2-dihydro-[1,6]naphthyridine-3-carbonyl)-amino]-4-methyl-benzoic acid methyl ester (from Example 28 supra) (2.5 g, 6.8 mmol) by following the method in Example 31. (Yield 2.0 g, 83%).

¹H NMR (300 MHz, d₆-DMSO) δ 11.80 (s, 1H), 8.99 (s, 1H), 8.91 (d, 2H, J=5.1 Hz), 7.62 (d, 1H, J=7.6 Hz), 7.37 (d, 1H, J=7.9 Hz), 6.63 (s, 1H), 3.95 (s, 3H), 2.40 (s, 3H). LC-MS: [M+H]⁺ 354.

### Example 30

### 4-Chloro-3-[(7-methoxy-2-oxo-1,2-dihydro-[1,6]naphthyridine-3-carbonyl)-amino]-benzoic acid methyl ester

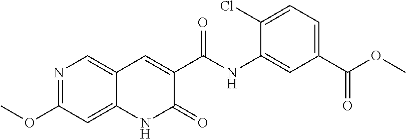

Triethylamine (1.16 g, 11.5 mmol) was added to a mixture of 7-methoxy-2-oxo-1,2-dihydro-[1,6]naphthyridine-3-carboxylic acid (from Example 15 supra) (2.3 g, 10.45 mmol) and HATU (4.37 g, 11.5 mmol) in dry DMF (30 mL). The resulting mixture was stirred at room temperature until clear solution was obtained. 3-Amino-4-chloro-benzoic acid methyl ester (2.32 g, 12.55 mmol) was added and the resulting mixture was stirred for another 20 hours. The reaction mixture was diluted with water (300 mL), aqueous saturated NaHCO₃ (60 mL) and ethyl acetate (150 mL). After thorough mixing, off white precipitate was collected by filtration, washed water, ethyl acetate and MeOH, and dried under reduced pressure to give 4-chloro-3-[(7-methoxy-2-oxo-1,2-dihydro-[1,6]naphthyridine-3-carbonyl)-amino]-benzoic acid methyl ester as an off-white solid. (Yield 2.9 g, 71.8%).

¹H NMR (300 MHz, d₆-DMSO) δ 12.42 (s, 1H), 9.18 (s, 1H), 9.01 (s, 1H), 8.91 (s, 1H), 7.70 (d, 2H, J=1.1 Hz), 6.61 (s, 1H), 3.95 (s, 3H), 3.89 (s, 3H). LC-MS: [M+H]⁺ 388.

### Example 31

### 4-Chloro-3-[(7-methoxy-2-oxo-1,2-dihydro-[1,6]naphthyridine-3-carbonyl)-amino]-benzoic acid

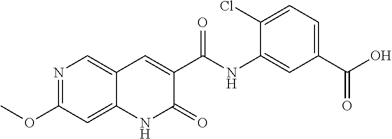

A mixture of 4-chloro-3-[(7-methoxy-2-oxo-1,2-dihydro-[1,6]naphthyridine-3-carbonyl)-amino]-benzoic acid methyl ester (from Example 30 supra) (2.5 g, 6.46 mmol) in MeOH (30 mL) and 4 N aqueous of NaOH (45 mL) was stirred at room temperature overnight. ¹H NMR showed that the methyl ester was consumed completely. Reaction mixture was then diluted with water (300 mL), acidified with 1 N aqueous HCl to pH 3-4. Pale yellow precipitate formed was collected by filtration, washed with water and dried under reduced pressure to give 4-chloro-3-[(7-methoxy-2-oxo-1,2-dihydro-[1,6]naphthyridine-3-carbonyl)-amino]-benzoic acid as a pale yellow solid. (2 g, 83%).

¹H NMR (300 MHz, d₆-DMSO) δ 12.55 (s, 1H), 12.33 (s, 1H), 9.15 (s, 1H), 9.01 (s, 1H), 8.92 (s, 1H), 7.67 (s, 2H), 6.63 (s, 1H), 3.95 (s, 3H). LC-MS: [M+H]⁺ 374.

### Example 32

### 7-Methoxy-2-oxo-1,2-dihydro-[1,6]naphthyridine-3-carboxylic acid (5-benzylcarbamoyl-2-chloro-phenyl)-amide

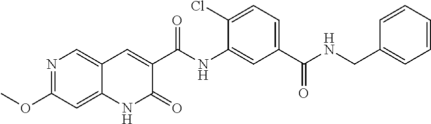

Triethylamine (42.4 mg, 0.42 mmol) was added to a mixture of 4-chloro-3-[(7-methoxy-2-oxo-1,2-dihydro-[1,6]naphthyridine-3-carbonyl)-amino]-benzoic acid (from Example 31 supra) (80 mg, 0.21 mmol) and HATU (97.6 mg, 0.26 mmol) in dry DMF (7 mL). The resulting solution was stirred at room temperature for 30 minutes with formation of white solid. Benzylamine (34.2 mg, 0.32 mmol) was added and the resulting solution was stirred for another 20 hours with formation of yellow solid. The reaction mixture was diluted with water (70 mL), aqueous saturated NaHCO₃ (20 mL) and ethyl acetate (30 mL). After thoroughly mixing, the precipitate was collected by filtration, washed with water, ethyl acetate and purified by refluxing with MeOH (25 mL) for 30 min. The precipitate was collected by filtration, dried under reduced pressure to give 7-methoxy-2-oxo-1,2-dihydro-[1,6]naphthyridine-3-carboxylic acid (5-benzylcarbamoyl-2-chloro-phenyl)-amide as a red-white solid. (Yield 50 mg, 50.5%).

¹H NMR (300 MHz, d₆-DMSO) δ 12.58 (s, 1H), 12.32 (s, 1H), 9.17 (t, 1H, J=5.9 Hz), 9.01 (d, 3H, J=27.2 Hz), 7.68 (s, 2H), 7.38-7.25 (m, 4H), 6.66 (s, 1H), 4.51 (d, 2H, J=5.9 Hz), 3.98 (s, 3H). LC-MS: [M+H]⁺ 462.8.

### Example 33

### 7-Methoxy-2-oxo-1,2-dihydro-[1,6]naphthyridine-3-carboxylic acid (5-benzylcarbamoyl-2-methyl-phenyl)-amide

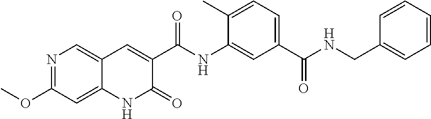

7-Methoxy-2-oxo-1,2-dihydro-[1,6]naphthyridine-3-carboxylic acid (5-benzylcarbamoyl-2-methyl-phenyl)-amide was prepared from 3-[(7-methoxy-2-oxo-1,2-dihydro-[1,6]naphthyridine-3-carbonyl)-amino]-4-methyl-benzoic acid (from Example 29 supra) (80 mg, 0.22 mmol) and benzylamine by following the method in Example 32. (Yield 60 mg, 60%).

¹H NMR (300 MHz, d₆-DMSO) δ 12.58 (s, 1H), 11.77 (s, 1H), 9.15-8.92 (m, 3H), 8.77 (s, 1H), 7.61 (dd, 1H, J=7.8 Hz, 1.3 Hz), 7.47-7.18 (m, 6H), 6.66 (s, 1H), 4.50 (d, 2H, J=5.9 Hz), 3.98 (s, 3H), 2.42 (s, 3H). LC-MS: [M+1]⁺ 442.9.

### Example 34

### 7-Methoxy-2-oxo-1,2-dihydro-[1,6]naphthyridine-3-carboxylic acid [5-(2-chloro-benzylcarbamoyl)-2-methyl-phenyl]-amide

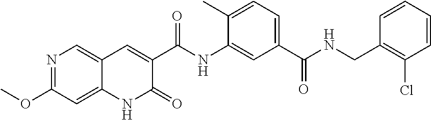

7-Methoxy-2-oxo-1,2-dihydro-[1,6]naphthyridine-3-carboxylic acid [5-(2-chloro-benzylcarbamoyl)-2-methyl-phenyl]-amide was prepared from 3-[(7-methoxy-2-oxo-1,2-dihydro-[1,6]naphthyridine-3-carbonyl)-amino]-4-methyl-benzoic acid (from Example 29 supra) (80 mg, 0.22 mmol) and 2-chloro-benzylamine by following the method in Example 32. (Yield 75 mg, 69%).

¹H NMR (300 MHz, d₆-DMSO) δ 12.57 (s, 1H), 11.79 (s, 1H), 9.14-8.92 (m, 3H), 8.80 (s, 1H), 7.72-7.59 (m, 1H), 7.57-7.26 (m, 5H), 6.66 (s, 1H), 4.57 (d, 2H, J=5.7 Hz), 3.98 (s, 3H), 2.43 (s, 3H). LC-MS: [M+H]⁺ 476.9.

### Example 35

### 7-Methoxy-2-oxo-1,2-dihydro-[1,6]naphthyridine-3-carboxylic acid [5-(3-chloro-benzylcarbamoyl)-2-methyl-phenyl]-amide

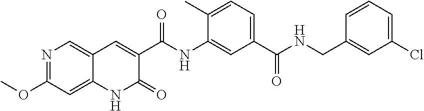

7-Methoxy-2-oxo-1,2-dihydro-[1,6]naphthyridine-3-carboxylic acid [5-(3-chloro-benzylcarbamoyl)-2-methyl-phenyl]-amide was prepared from 3-[(7-methoxy-2-oxo-1,2-dihydro-[1,6]naphthyridine-3-carbonyl)-amino]-4-methyl-benzoic acid (from Example 29 supra) (80 mg, 0.22 mmol) and 3-chloro-benzylamine by following the method in Example 32. (Yield 55 mg, 51%).

¹H NMR (300 MHz, d₆-DMSO) δ 12.57 (s, 1H), 11.78 (s, 1H), 9.02 (t, 3H, J=15.3 Hz), 8.78 (s, 1H), 7.61 (d, 1H, J=7.8 Hz), 7.53-7.22 (m, 5H), 6.66 (s, 1H), 4.50 (d, 2H, J=5.8 Hz), 3.98 (s, 3H), 2.42 (s, 3H). LC-MS: [M+H]⁺ 476.9.

### Example 36

### 7-Methoxy-2-oxo-1,2-dihydro-[1,6]naphthyridine-3-carboxylic acid [5-(4-chloro-benzylcarbamoyl)-2-methyl-phenyl]-amide

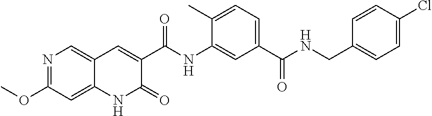

7-Methoxy-2-oxo-1,2-dihydro-[1,6]naphthyridine-3-carboxylic acid [5-(4-chloro-benzylcarbamoyl)-2-methyl-phenyl]-amide was prepared from 3-[(7-methoxy-2-oxo-1,2-dihydro-[1,6]naphthyridine-3-carbonyl)-amino]-4-methyl-benzoic acid (from Example 29 supra) (80 mg, 0.22 mmol) and 4-chloro-benzylamine by following the method in Example 32. (Yield 65 mg, 60%).

¹H NMR (300 MHz, d₆-DMSO) δ 12.58 (s, 1H), 11.78 (s, 1H), 9.02 (t, 3H, J=15.0 Hz), 8.76 (s, 1H), 7.66-7.55 (m, 1H), 7.39 (q, 5H, J=8.6 Hz), 6.66 (s, 1H), 4.48 (d, 2H, J=5.8 Hz), 3.98 (s, 3H), 2.42 (s, 3H). LC-MS: [M+H]⁺ 476.9.

### Example 37

### 7-Methoxy-2-oxo-1,2-dihydro-[1,6]naphthyridine-3-carboxylic acid {2-methyl-5-[(thiophen-2-ylmethyl)-carbamoyl]-phenyl}-amide

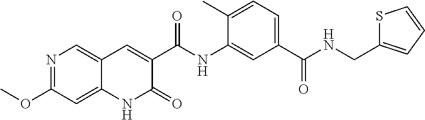

7-Methoxy-2-oxo-1,2-dihydro-[1,6]naphthyridine-3-carboxylic acid {2-methyl-5-[(thiophen-2-ylmethyl)-carbamoyl]-phenyl}-amide was prepared from 3-[(7-methoxy-2-oxo-1,2-dihydro-[1,6]naphthyridine-3-carbonyl)-amino]-4-methyl-benzoic acid (from Example 29 supra) (80 mg, 0.22 mmol) and thiophen-2-yl-methylamine by following the method in Example 32. (Yield 25 mg, 24%).

¹H NMR (300 MHz, d₆-DMSO) δ 12.58 (s, 1H), 11.77 (s, 1H), 9.21-8.90 (m, 3H), 8.76 (d, 1H, J=1.4 Hz), 7.58 (dd, 1H, J=7.9 Hz, 1.6 Hz), 7.47-7.29 (m, 2H), 7.10-6.89 (m, 2H), 6.66 (s, 1H), 4.65 (d, 2H, J=5.8 Hz), 3.98 (s, 3H), 2.41 (s, 3H). LC-MS: [M+H]⁺ 448.8.

### Example 38

### 7-Methoxy-2-oxo-1,2-dihydro-[1,6]naphthyridine-3-carboxylic acid {2-methyl-5-[(thiophen-3-ylmethyl)-carbamoyl]-phenyl}-amide

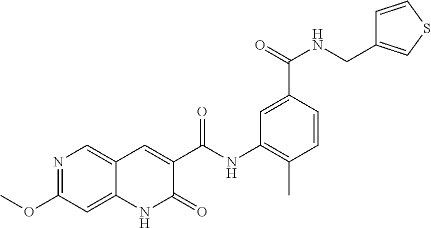

7-Methoxy-2-oxo-1,2-dihydro-[1,6]naphthyridine-3-carboxylic acid {2-methyl-5-[(thiophen-3-ylmethyl)-carbamoyl]-phenyl}-amide was prepared from 3-[(7-methoxy-2-oxo-1,2-dihydro-[1,6]naphthyridine-3-carbonyl)-amino]-4-methyl-benzoic acid (from Example 29 supra) (80 mg, 0.22 mmol) and thiophen-3-yl-methylamine by following the method in Example 32. (Yield 70 mg, 69%).

¹H NMR (300 MHz, d₆-DMSO) δ 12.56 (s, 1H), 11.76 (s, 1H), 8.96 (t, 3H, J=15.0 Hz), 8.74 (s, 1H), 7.65-7.43 (m, 2H), 7.41-7.26 (m, 2H), 7.10 (d, 1H, J=4.9 Hz), 6.64 (s, 1H), 4.46 (d, 2H, J=5.8 Hz), 3.96 (s, 3H), 2.39 (s, 3H). LC-MS: [M+H]⁺ 448.7.

### Example 39

### 7-Methoxy-2-oxo-1,2-dihydro-[1,6]naphthyridine-3-carboxylic acid [2-chloro-5-(2-chloro-benzylcarbamoyl)-phenyl]-amide

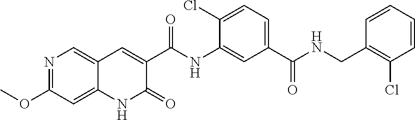

7-Methoxy-2-oxo-1,2-dihydro-[1,6]naphthyridine-3-carboxylic acid [2-chloro-5-(2-chloro-benzylcarbamoyl)-phenyl]-amide was prepared from 4-chloro-3-[(7-methoxy-2-oxo-1,2-dihydro-[1,6]naphthyridine-3-carbonyl)-amino]-benzoic acid (from Example 31 supra) (80 mg, 0.21 mmol) and 2-chloro-benzylamine by following the method in Example 32. (Yield 55 mg, 52%). ¹H NMR (300 MHz, d₆-DMSO) δ 12.46 (s, 1H), 12.32 (s, 1H), 9.16 (t, 1H, J=5.7 Hz), 9.06 (d, 2H, J=4.3 Hz), 8.96 (s, 1H), 7.70 (s, 2H), 7.53-7.44 (m, 1H), 7.43-7.27 (m, 3H), 6.65 (s, 1H), 4.56 (d, 2H, J=5.6 Hz), 3.97 (s, 3H). LC-MS: [M+H]⁺ 496.8.

### Example 40

### 7-Methoxy-2-oxo-1,2-dihydro-[1,6]naphthyridine-3-carboxylic acid [2-chloro-5-(3-chloro-benzylcarbamoyl)-phenyl]-amide

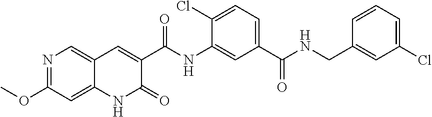

7-Methoxy-2-oxo-1,2-dihydro-[1,6]naphthyridine-3-carboxylic acid [2-chloro-5-(3-chloro-benzylcarbamoyl)-phenyl]-amide was prepared from 4-chloro-3-[(7-methoxy-2-oxo-1,2-dihydro-[1,6]naphthyridine-3-carbonyl)-amino]-benzoic acid (from Example 31 supra) (80 mg, 0.21 mmol) and 3-chloro-benzylamine by following the method in Example 32. (Yield 50 mg, 47%). ¹H NMR (300 MHz, d₆-DMSO) δ 12.56 (s, 1H), 12.31 (s, 1H), 9.19 (t, 1H, J=5.8 Hz), 9.05 (s, 2H), 8.96 (s, 1H), 7.68 (s, 2H), 7.47-7.26 (m, 4H), 6.65 (s, 1H), 4.49 (d, 2H, J=5.8 Hz), 3.97 (s, 3H). LC-MS: [M+H]⁺ 496.8.

### Example 41

### 7-Methoxy-2-oxo-1,2-dihydro-[1,6]naphthyridine-3-carboxylic acid [2-chloro-5-(4-chloro-benzylcarbamoyl)-phenyl]-amide

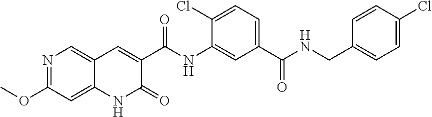

7-Methoxy-2-oxo-1,2-dihydro-[1,6]naphthyridine-3-carboxylic acid [2-chloro-5-(4-chloro-benzylcarbamoyl)-phenyl]-amide was prepared from 4-chloro-3-[(7-methoxy-2-oxo-1,2-dihydro-[1,6]naphthyridine-3-carbonyl)-amino]-benzoic acid (from Example 31 supra) (80 mg, 0.21 mmol) and 4-chloro-benzylamine by following the method in Example 32. (Yield 70 mg, 66%). ¹H NMR (300 MHz, d₆-DMSO) δ 12.59 (s, 1H), 12.32 (s, 1H), 9.20 (t, 1H, J=5.8 Hz), 9.06 (s, 2H), 8.97 (s, 1H), 7.73-7.63 (m, 2H), 7.40 (q, 4H, J=8.5 Hz), 6.66 (s, 1H), 4.49 (d, 2H, J=5.8 Hz), 3.99 (s, 3H). LC-MS: [M+H]⁺ 496.8.

### Example 42

### 7-Methoxy-2-oxo-1,2-dihydro-[1,6]naphthyridine-3-carboxylic acid {2-chloro-5-[(thiophen-2-ylmethyl)-carbamoyl]-phenyl}-amide

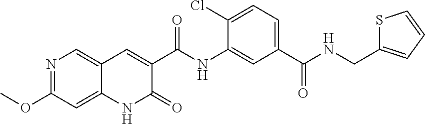

7-Methoxy-2-oxo-1,2-dihydro-[1,6]naphthyridine-3-carboxylic acid {2-chloro-5-[(thiophen-2-ylmethyl)-carbamoyl]-phenyl}-amide was prepared from 4-chloro-3-[(7-methoxy-2-oxo-1,2-dihydro-[1,6]naphthyridine-3-carbonyl)-amino]-benzoic acid (from Example 31 supra) (80 mg, 0.21 mmol) and thiophen-2-yl-methylamine by following the method in Example 32. (Yield 45 mg, 45%). ¹H NMR (300 MHz, d₆-DMSO) δ 12.58 (s, 1H), 12.32 (s, 1H), 9.26 (t, 1H, J=5.8 Hz), 9.03 (dd, 3H, J=14.4 Hz, 12.7 Hz), 7.65 (dt, 2H, J=8.4 Hz), 7.50-7.33 (m, 1H), 7.14-6.93 (m, 2H), 6.66 (s, 1H), 4.66 (d, 2H, J=5.7 Hz), 3.98 (s, 3H).

LC-MS: [M+H]⁺ 468.8.

### Example 43

### 7-Methoxy-2-oxo-1,2-dihydro-[1,6]naphthyridine-3-carboxylic acid {2-chloro-5-[(thiophen-3-ylmethyl)-carbamoyl]-phenyl}-amide

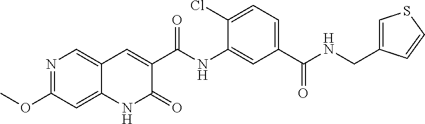

7-Methoxy-2-oxo-1,2-dihydro-[1,6]naphthyridine-3-carboxylic acid {2-chloro-5-[(thiophen-3-ylmethyl)-carbamoyl]-phenyl}-amide was prepared from 4-chloro-3-[(7-methoxy-2-oxo-1,2-dihydro-[1,6]naphthyridine-3-carbonyl)-amino]-benzoic acid (from Example 31 supra) (80 mg, 0.21 mmol) and thiophen-3-yl-methylamine by following the method in Example 32. (Yield 80 mg, 80%). ¹H NMR (300 MHz, d₆-DMSO) δ 12.56 (s, 1H), 12.31 (s, 1H), 9.22-8.89 (m, 4H), 7.79-7.59 (m, 2H), 7.57-7.45 (m, 1H), 7.36 (s, 1H), 7.12 (d, 1H, J=4.9 Hz), 6.66 (s, 1H), 4.50 (d, 2H, J=5.7 Hz), 3.98 (s, 3H). LC-MS: [M+H]⁺ 468.8.

### Example 44

### (2-{4-Chloro-3-[(7-methoxy-2-oxo-1,2-dihydro-[1,6]naphthyridine-3-carbonyl)-amino]-benzoylamino}-2-phenyl-ethyl)-carbamic acid tert-butyl ester

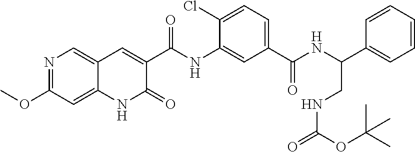

(2-{4-Chloro-3-[(7-methoxy-2-oxo-1,2-dihydro-[1,6]naphthyridine-3-carbonyl)-amino]-benzoylamino}-2-phenyl-ethyl)-carbamic acid tert-butyl ester was prepared from 4-chloro-3-[(7-methoxy-2-oxo-1,2-dihydro-[1,6]naphthyridine-3-carbonyl)-amino]-benzoic acid (from Example 31 supra) (100 mg, 0.27 mmol) and (2-amino-2-phenyl-ethyl)-carbamic acid tert-butyl ester (from Example 1 supra) by following the method in Example 32. (Yield 100 mg, 63%).

LC-MS: [M-100+1]⁺ 492.

### Example 45

### 7-Methoxy-2-oxo-1,2-dihydro-[1,6]naphthyridine-3-carboxylic acid [5-(2-amino-1-phenyl-ethylcarbamoyl)-2-chloro-phenyl]-amide; hydrochloride

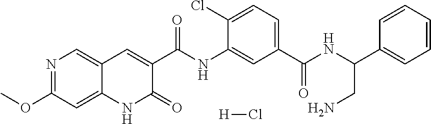

A mixture of (2-{4-chloro-3-[(7-methoxy-2-oxo-1,2-dihydro-[1,6]naphthyridine-3-carbonyl)-amino]-benzoylamino}-2-phenyl-ethyl)-carbamic acid tert-butyl ester (from Example 44 supra) (100 mg, 0.17 mmol) in 5 N HCl (15 mL) in dioxane was stirred at room temperature for 20 hours. Then it was concentrated under reduced pressure and the residue was diluted with ethyl acetate (20 mL). The precipitate was collected by filtration. This crude product was purified by refluxing in MeOH (25 mL) for 30 minutes and the precipitate was collected by filtration, dried under reduced pressure to give 7-methoxy-2-oxo-1,2-dihydro-[1,6]naphthyridine-3-carboxylic acid [5-(2-amino-1-phenyl-ethylcarbamoyl)-2-chloro-phenyl]-amide hydrochloride as a pale yellow solid. (Yield 55 mg, 57.7%).

¹H NMR (300 MHz, d₆-DMSO) δ 12.32 (s, 1H), 9.16 (d, 1H, J=8.3 Hz), 9.09-8.93 (m, 3H), 8.14 (s, 2H), 7.79 (dd, 2H, J=32.4 Hz, 8.4 Hz), 7.58-7.28 (m, 5H), 6.68 (s, 1H), 5.39 (t, 1H, J=7.4 Hz), 3.98 (s, 3H), 3.44-3.36 (m, 1H), 3.29-3.17 (m, 1H). LC-MS: [M+H]⁺ 491.7.

### Example 46

### 3-({7-[(2,2-Dimethyl-[1,3]dioxolan-4-ylmethyl)-amino]-2-oxo-1,2-dihydro-[1,6]naphthyridine-3-carbonyl}-amino)-benzoic acid methyl ester

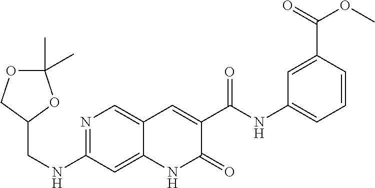

To a stirred solution of 7-[(2,2-dimethyl-[1,3]dioxolan-4-ylmethyl)-amino]-2-oxo-1,2-dihydro-[1,6]naphthyridine-3-carboxylic acid (110 mg, 0.344 mmol) (from Example 27 supra) and methyl 3-aminobenzoate (62 mg, 0.41 mmol) in THF (20 mL) was added HATU (157 mg, 0.413 mmol) and DIEA (133 mg, 1.03 mmol) at room temperature under nitrogen. The reaction mixture was stirred at this temperature for sixteen hours. The solvent was removed under reduced pressure and the residue was treated with methanol (20 mL), then stirred at room temperature for 20 minutes, filtered and washed with methanol (10 mL), ethyl acetate (10 mL), dichloromethane (20 mL) and dried to give 3-({7-[(2,2-dimethyl-[1,3]dioxolan-4-ylmethyl)-amino]-2-oxo-1,2-dihydro-[1,6]naphthyridine-3-carbonyl}-amino)-benzoic acid methyl ester as a white solid which was used directly in the next step without further purification. (Yield 110 mg, 45%). LC-MS: [M+H]⁺ 453.1.

### Example 47

### 3-{[7-(2,3-Dihydroxy-propylamino)-2-oxo-1,2-dihydro-[1,6]naphthyridine-3-carbonyl]-amino}-benzoic acid methyl ester; hydrochloride

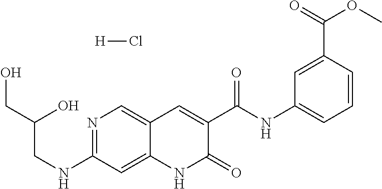

To a stirred solution of 3-({7-[(2,2-dimethyl-[1,3]dioxolan-4-ylmethyl)-amino]-2-oxo-1,2-dihydro-[1,6]naphthyridine-3-carbonyl}-amino)-benzoic acid methyl ester (10 mg, 0.022 mmol) (from Example 46 supra) in methanol (10 mL) was added conc. HCl (four drops) at room temperature. The reaction mixture was stirred at this temperature for 16 hours. The solvent was removed under reduced pressure and the residue was treated with dichloromethane (5 mL), methanol (5 mL), and then filtered to give crude product as a solid. It was purified by prep-HPLC to give 3-{[7-(2,3-dihydroxy-propylamino)-2-oxo-1,2-dihydro-[1,6]naphthyridine-3-carbonyl]-amino}-benzoic acid methyl ester; hydrochloride as a yellow solid. (Yield 8 mg, 81.0%).

¹H NMR (300 MHz, DMSO+D₂O): δ 8.67 (s, 1H), 8.56 (s, 1H), 8.28 (s, 1H), 7.74 (d, 1H, J=9.3 Hz), 7.65 (d, 1H, J=7.5 Hz), 7.46 (t, 1H, J=7.9 Hz), 6.24 (s, 1H), 3.86 (s, 3H), 3.67-3.63 (m, 1H), 3.41-3.35 (d, 3H), 3.22-3.15 (m, 1H). LC-MS: [M+H]⁺ 413.0.

### Example 48

### 3-({7-[(2,2-Dimethyl-[1,3]dioxolan-4-ylmethyl)-amino]-2-oxo-1,2-dihydro-[1,6]naphthyridine-3-carbonyl}-amino)-4-methyl-benzoic acid methyl ester

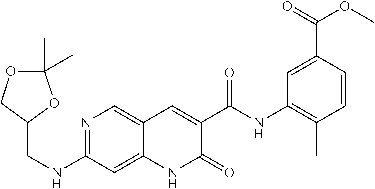

3-({7-[(2,2-Dimethyl-[1,3]dioxolan-4-ylmethyl)-amino]-2-oxo-1,2-dihydro-[1,6]naphthyridine-3-carbonyl}-amino)-4-methyl-benzoic acid methyl ester was prepared from 7-[(2,2-dimethyl-[1,3]dioxolan-4-ylmethyl)-amino]-2-oxo-1,2-dihydro-[1,6]naphthyridine-3-carboxylic acid (from Example 27 supra) (130 mg, 0.41 mmol) and 3-amino-4-methyl-benzoic acid methyl ester by following the method in Example 46. (Yield 150 mg, 80.4%).

LC-MS: [M+H]⁺ 467.1.

### Example 49

### 3-{[7-(2,3-Dihydroxy-propylamino)-2-oxo-1,2-dihydro-[1,6]naphthyridine-3-carbonyl]-amino}-4-methyl-benzoic acid methyl ester; hydrochloride

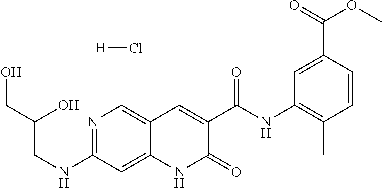

3-{[7-(2,3-Dihydroxy-propylamino)-2-oxo-1,2-dihydro-[1,6]naphthyridine-3-carbonyl]-amino}-4-methyl-benzoic acid methyl ester; hydrochloride was prepared from 3-({7-[(2,2-dimethyl-[1,3]dioxolan-4-ylmethyl)-amino]-2-oxo-1,2-dihydro-[1,6]naphthyridine-3-carbonyl}-amino)-4-methyl-benzoic acid methyl ester (from Example 48 supra) (0.15 g, 0.32 mmol) by following the method in Example 47. (Yield 0.055 g, 37.1%).

¹H NMR (300 MHz, DMSO+D₂O): δ 8.89-8.88 (m, 1H), 8.77 (s, 1H), 8.67 (s, 1H), 7.60 (dd, 1H, J₁=9.9 Hz, J₂=1.5 Hz), 7.39 (d, 1H, J=8.1 Hz), 6.36 (s, 1H), 3.82 (s, 3H), 3.70-3.67 (m, 1H), 3.41-3.68 (m, 3H), 3.27-3.20 (m, 1H). LC-MS: [M+H]⁺ 427.1.

### Example 50

### [2-{4-Chloro-3-[(7-methoxy-2-oxo-1,2-dihydro-[1,6]naphthyridine-3-carbonyl)-amino]-benzoylamino}-2-(3-chloro-phenyl)-ethyl]-carbamic acid tert-butyl ester

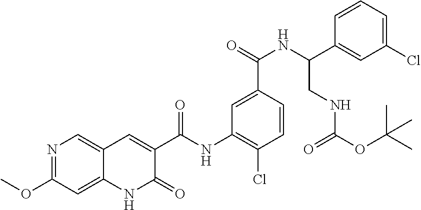

[2-{4-Chloro-3-[(7-methoxy-2-oxo-1,2-dihydro-[1,6]naphthyridine-3-carbonyl)-amino]-benzoylamino}-2-(3-chloro-phenyl)-ethyl]-carbamic acid tert-butyl ester was prepared from 4-chloro-3-[(7-methoxy-2-oxo-1,2-dihydro-[1,6]naphthyridine-3-carbonyl)-amino]-benzoic acid (from Example 31 supra) (100 mg, 0.27 mmol) and [2-amino-2-(3-chloro-phenyl)-ethyl]-carbamic acid tert-butyl ester (from Example 3 supra) by following the method in Example 32. (Yield 85 mg, 51%). LC-MS: [M-100+H]⁺ 526.

### Example 51

### 7-Methoxy-2-oxo-1,2-dihydro-[1,6]naphthyridine-3-carboxylic acid {5-[2-amino-1-(3-chloro-phenyl)-ethylcarbamoyl]-2-chloro-phenyl}-amide; hydrochloride

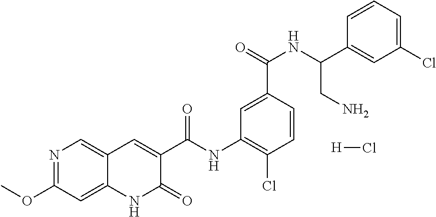

7-Methoxy-2-oxo-1,2-dihydro-[1,6]naphthyridine-3-carboxylic acid {5-[2-amino-1-(3-chloro-phenyl)-ethylcarbamoyl]-2-chloro-phenyl}-amide; hydrochloride was prepared from [2-{4-chloro-3-[(7-methoxy-2-oxo-1,2-dihydro-[1,6]naphthyridine-3-carbonyl)-amino]-benzoylamino}-2-(3-chloro-phenyl)-ethyl]-carbamic acid tert-butyl ester (from Example 50 supra) (85 mg, 0.16 mmol) by following the method in Example 45. (Yield 33 mg, 40.6%).

¹H NMR (300 MHz, d₆-DMSO) δ 12.33 (s, 1H), 9.20 (d, 1H, J=8.2 Hz), 9.03 (dd, 3H, J=12.9 Hz, 10.9 Hz), 8.15 (s, 2H), 7.92-7.70 (m, 2H), 7.59-7.33 (m, 4H), 6.68 (s, 1H), 5.46-5.27 (m, 1H), 3.98 (s, 3H), 3.26 (dd, 2H, J=12.8 Hz, 4.3 Hz). LC-MS: [M+H]⁺ 525.7.

### Example 52

### (3-{4-Chloro-3-[(7-methoxy-2-oxo-1,2-dihydro-[1,6]naphthyridine-3-carbonyl)-amino]-benzoylamino}-3-thiophen-3-yl-propyl)-carbamic acid tert-butyl ester

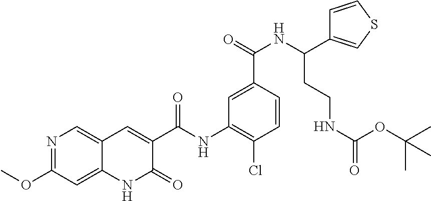

(3-{4-Chloro-3-[(7-methoxy-2-oxo-1,2-dihydro-[1,6]naphthyridine-3-carbonyl)-amino]-benzoylamino}-3-thiophen-3-yl-propyl)-carbamic acid tert-butyl ester was prepared from 4-chloro-3-[(7-methoxy-2-oxo-1,2-dihydro-[1,6]naphthyridine-3-carbonyl)-amino]-benzoic acid (from Example 31 supra) (100 mg, 0.27 mmol) and (3-amino-3-thiophen-3-yl-propyl)-carbamic acid tert-butyl ester (from Example 4 supra) by following the method in Example 32. (Yield 110 mg, 67%). LC-MS: [M-100+1]⁺ 512.

### Example 53

### 7-Methoxy-2-oxo-1,2-dihydro-[1,6]naphthyridine-3-carboxylic acid [5-(3-amino-1-thiophen-3-yl-propylcarbamoyl)-2-chloro-phenyl]-amide

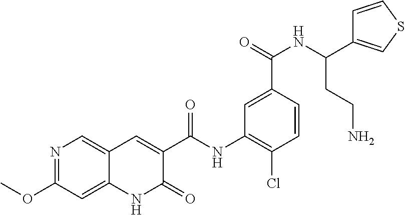

7-Methoxy-2-oxo-1,2-dihydro-[1,6]naphthyridine-3-carboxylic acid [5-(3-amino-1-thiophen-3-yl-propylcarbamoyl)-2-chloro-phenyl]-amide was prepared from (3-{4-chloro-3-[(7-methoxy-2-oxo-1,2-dihydro-[1,6]naphthyridine-3-carbonyl)-amino]-benzoylamino}-3-thiophen-3-yl-propyl)-carbamic acid tert-butyl ester (from Example 52 supra) (110 mg, 0.18 mmol) by following the method in Example 45. The crude product obtained was purified by prep-HPLC (Instrument: Gilson 281, Column. Gemini 5u C18 250×21.5 mm, Mobile Phase: CH₃CN/H₂O (0.1% TFA), 25/75 to 90/10, flow rate 20 mL/min). The resulting solution of product was evaporated, suspended in saturated aqueous NaHCO₃ (10 mL) and stirred for 30 minutes. The solid was collected by filtration and dried to give 7-methoxy-2-oxo-1,2-dihydro-[1,6]naphthyridine-3-carboxylic acid [5-(3-amino-1-thiophen-3-yl-propylcarbamoyl)-2-chloro-phenyl]-amide. (Yield 13 mg, 14%).

¹H NMR (300 MHz, d₆-DMSO) δ 12.76 (s, 1H), 12.42 (s, 1H), 9.28-8.98 (m, 4H), 8.13 (s, 3H), 7.89-7.50 (m, 4H), 7.32 (d, 1H, J=4.9 Hz), 6.79 (s, 1H), 5.41 (q, 1H, J=7.5 Hz), 4.07 (s, 3H), 3.06-2.72 (m, 2H), 2.30 (m, 2H). LC-MS: [M+H]⁺ 512.1.

### Example 54

### (2-{3-[(7-Methoxy-2-oxo-1,2-dihydro-[1,6]naphthyridine-3-carbonyl)-amino]-4-methyl-benzoylamino}-2-phenyl-ethyl)-carbamic acid tert-butyl ester

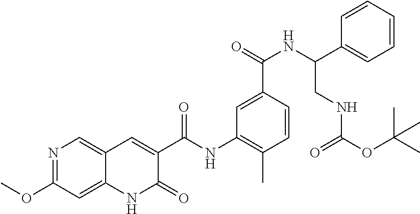

(2-{3-[(7-Methoxy-2-oxo-1,2-dihydro-[1,6]naphthyridine-3-carbonyl)-amino]-4-methyl-benzoylamino}-2-phenyl-ethyl)-carbamic acid tert-butyl ester was prepared from 3-[(7-methoxy-2-oxo-1,2-dihydro-[1,6]naphthyridine-3-carbonyl)-amino]-4-methyl-benzoic acid (from Example 29 supra) (100 mg, 0.28 mmol) and (2-amino-2-phenyl-ethyl)-carbamic acid tert-butyl ester (from Example 1 supra) by following the method in Example 32. (Yield 70 mg, 43%).

LC-MS: [M+H]⁺ 572.

### Example 55

### 7-Methoxy-2-oxo-1,2-dihydro-[1,6]naphthyridine-3-carboxylic acid [5-(2-amino-1-phenyl-ethylcarbamoyl)-2-methyl-phenyl]-amide; hydrochloride

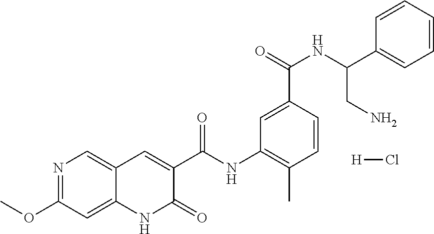

7-Methoxy-2-oxo-1,2-dihydro-[1,6]naphthyridine-3-carboxylic acid [5-(2-amino-1-phenyl-ethylcarbamoyl)-2-methyl-phenyl]-amide was prepared from (2-{3-[(7-methoxy-2-oxo-1,2-dihydro-[1,6]naphthyridine-3-carbonyl)-amino]-4-methyl-benzoylamino}-2-phenyl-ethyl)-carbamic acid tert-butyl ester (from Example 54 supra) (70 mg, 0.12 mmol) by following the method in Example 45. (Yield 16 mg, 24%).

¹H NMR (300 MHz, d₆-DMSO) δ 11.74 (s, 1H), 9.06-8.83 (m, 3H), 8.71 (s, 1H), 8.11 (s, 2H), 7.72 (d, 1H, J=7.9 Hz), 7.54-7.19 (m, 6H), 6.65 (s, 1H), 5.44-5.24 (m, 1H), 3.94 (s, 3H), 3.37 (s, 1H), 3.17 (dd, 1H, J=12.8 Hz, 3.8 Hz), 2.38 (s, 3H). LC-MS: [M+H]⁺ 471.8.

### Example 56

### (2-(3-Chloro-phenyl)-2-{3-[(7-methoxy-2-oxo-1,2-dihydro-[1,6]naphthyridine-3-carbonyl)-amino]-4-methyl-benzoylamino}-ethyl)-carbamic acid tert-butyl ester

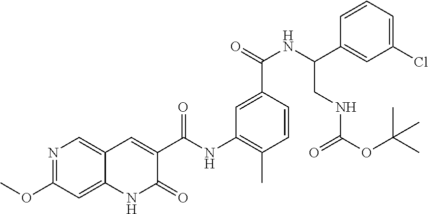

(2-(3-Chloro-phenyl)-2-{3-[(7-methoxy-2-oxo-1,2-dihydro-[1,6]naphthyridine-3-carbonyl)-amino]-4-methyl-benzoylamino}-ethyl)-carbamic acid tert-butyl ester was prepared from 3-[(7-methoxy-2-oxo-1,2-dihydro-[1,6]naphthyridine-3-carbonyl)-amino]-4-methyl-benzoic acid (from Example 29 supra) (100 mg, 0.28 mmol) and [2-amino-2-(3-chloro-phenyl)-ethyl]-carbamic acid tert-butyl ester (from Example 3 supra) by following the method in Example 32. (Yield 160 mg, 93.6%).

LC-MS: [M-100+1]⁺ 506.

### Example 57

### 7-Methoxy-2-oxo-1,2-dihydro-[1,6]naphthyridine-3-carboxylic acid {5-[2-amino-1-(3-chloro-phenyl)-ethylcarbamoyl]-2-methyl-phenyl}-amide; hydrochloride

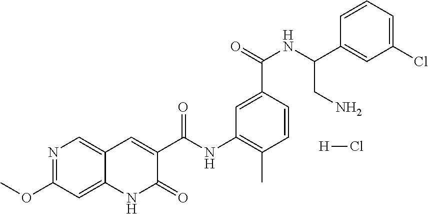

7-Methoxy-2-oxo-1,2-dihydro-[1,6]naphthyridine-3-carboxylic acid {5-[2-amino-1-(3-chloro-phenyl)-ethylcarbamoyl]-2-methyl-phenyl}-amide was prepared from (2-(3-chloro-phenyl)-2-{3-[(7-methoxy-2-oxo-1,2-dihydro-[1,6]naphthyridine-3-carbonyl)-amino]-4-methyl-benzoylamino}-ethyl)-carbamic acid tert-butyl ester (from Example 56 supra) (160 mg, 0.26 mmol) by following the method in Example 45. (Yield 100 mg, 65.5%).

¹H NMR (300 MHz, d₆-DMSO) δ 12.67 (s, 1H), 11.77 (s, 1H), 9.14 (d, 1H, J=8.0 Hz), 8.97 (d, 2H, J=20.1 Hz), 8.73 (s, 1H), 8.30 (s, 3H), 7.81 (d, 1H, J=7.9 Hz), 7.42-7.40 (m, 5H), 6.66 (d, 4H, J=21.2 Hz), 5.46-5.30 (m, 1H), 3.96 (s, 3H), 3.48-3.11 (m, 2H), 2.40 (s, 3H). LC-MS: [M+H]⁺ 505.7.

### Example 58

### (2-{3-[(7-Methoxy-2-oxo-1,2-dihydro-[1,6]naphthyridine-3-carbonyl)-amino]-4-methyl-benzoylamino}-2-thiophen-3-yl-ethyl)-carbamic acid tert-butyl ester

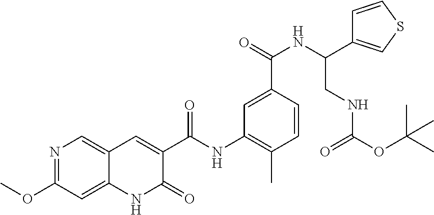

(2-{3-[(7-Methoxy-2-oxo-1,2-dihydro-[1,6]naphthyridine-3-carbonyl)-amino]-4-methyl-benzoylamino}-2-thiophen-3-yl-ethyl)-carbamic acid tert-butyl ester was prepared from 3-[(7-methoxy-2-oxo-1,2-dihydro-[1,6]naphthyridine-3-carbonyl)-amino]-4-methyl-benzoic acid (from Example 29 supra) (100 mg, 0.28 mmol) and (2-amino-2-thiophen-3-yl-ethyl)-carbamic acid tert-butyl ester (from Example 5 supra) by following the method in Example 32. (Yield 100 mg, 61%). LC-MS: [M-100+1]⁺ 478.

### Example 59

### 7-Methoxy-2-oxo-1,2-dihydro-[1,6]naphthyridine-3-carboxylic acid [5-(2-amino-1-thiophen-3-yl-ethylcarbamoyl)-2-methyl-phenyl]-amide; hydrochloride

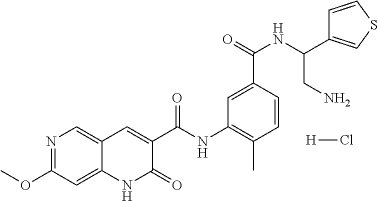

7-Methoxy-2-oxo-1,2-dihydro-[1,6]naphthyridine-3-carboxylic acid [5-(2-amino-1-thiophen-3-yl-ethylcarbamoyl)-2-methyl-phenyl]-amide; hydrochloride was prepared from (2-{3-[(7-methoxy-2-oxo-1,2-dihydro-[1,6]naphthyridine-3-carbonyl)-amino]-4-methyl-benzoylamino}-2-thiophen-3-yl-ethyl)-carbamic acid tert-butyl ester (from Example 58 supra) (100 mg, 0.17 mmol) by following the method in Example 45. (Yield 47 mg, 49.4%).

¹H NMR (300 MHz, d₆-DMSO) δ 12.66 (s, 1H), 11.77 (s, 1H), 9.08-8.88 (m, 3H), 8.74 (s, 1H), 8.20 (s, 3H), 7.74 (dd, 1H, J=7.9 Hz, 1.2 Hz), 7.63-7.33 (m, 3H), 7.20 (d, 1H, J=4.2 Hz), 6.70 (s, 1H), 5.49 (m, 4.5, 1H), 3.96 (s, 3H), 3.31 (m, 2H), 2.40 (s, 3H). LC-MS: [M+H]⁺ 477.8.

### Example 60

### (3-{3-[(7-Methoxy-2-oxo-1,2-dihydro-[1,6]naphthyridine-3-carbonyl)-amino]-4-methyl-benzoylamino}-3-phenyl-propyl)-carbamic acid tert-butyl ester

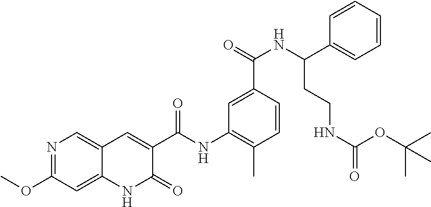

(3-{3-[(7-Methoxy-2-oxo-1,2-dihydro-[1,6]naphthyridine-3-carbonyl)-amino]-4-methyl-benzoylamino}-3-phenyl-propyl)-carbamic acid tert-butyl ester was prepared from 3-[(7-methoxy-2-oxo-1,2-dihydro-[1,6]naphthyridine-3-carbonyl)-amino]-4-methyl-benzoic acid (from Example 29 supra) (100 mg, 0.28 mmol) and (3-amino-3-phenyl-propyl)-carbamic acid tert-butyl ester (from Example 2 supra) by following the method in Example 32. (Yield 120 mg, 72.7%). LC-MS: [M+H]⁺ 586.

### Example 61

### 7-Methoxy-2-oxo-1,2-dihydro-[1,6]naphthyridine-3-carboxylic acid [5-(3-amino-1-phenyl-propylcarbamoyl)-2-methyl-phenyl]-amide; hydrochloride

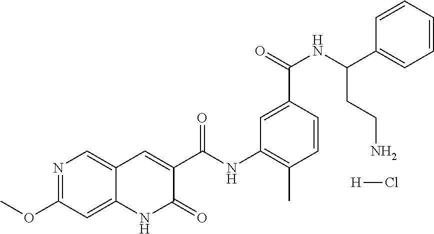

7-Methoxy-2-oxo-1,2-dihydro-[1,6]naphthyridine-3-carboxylic acid [5-(3-amino-1-phenyl-propylcarbamoyl)-2-methyl-phenyl]-amide; hydrochloride was prepared from (3-{3-[(7-methoxy-2-oxo-1,2-dihydro-[1,6]naphthyridine-3-carbonyl)-amino]-4-methyl-benzoylamino}-3-phenyl-propyl)-carbamic acid tert-butyl ester (from Example 60 supra) (120 mg, 0.20 mmol) by following the method in Example 45. (Yield 80 mg, 70%).

¹H NMR (300 MHz, d₆-DMSO) δ 12.63 (s, 1H), 11.74 (s, 1H), 8.94 (t, 3H, J=14.6 Hz), 8.69 (s, 1H), 8.03 (s, 3H), 7.61 (d, 1H, J=7.9 Hz), 7.48-7.14 (m, 6H), 6.68 (s, 1H), 5.14 (q, 1H, J=8.1 Hz), 3.94 (s, 3H), 2.77 (m, 3H), 2.37 (s, 3H), 2.30-2.01 (m, 2H). LC-MS: [M+H]⁺ 485.9.

### Example 62

### (3-{3-[(7-Methoxy-2-oxo-1,2-dihydro-[1,6]naphthyridine-3-carbonyl)-amino]-4-methyl-benzoylamino}-3-thiophen-3-yl-propyl)-carbamic acid tert-butyl ester

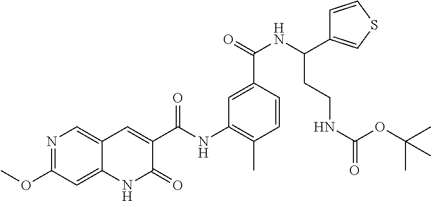

(3-{3-[(7-Methoxy-2-oxo-1,2-dihydro-[1,6]naphthyridine-3-carbonyl)-amino]-4-methyl-benzoylamino}-3-thiophen-3-yl-propyl)-carbamic acid tert-butyl ester was prepared from 3-[(7-methoxy-2-oxo-1,2-dihydro-[1,6]naphthyridine-3-carbonyl)-amino]-4-methyl-benzoic acid (from Example 29 supra) (100 mg, 0.28 mmol) and (3-amino-3-thiophen-3-yl-propyl)-carbamic acid tert-butyl ester (from Example 4 supra) by following the method in Example 32. (Yield 100 mg, 60%). LC-MS: [M+H]⁺ 592.

### Example 63

### 7-Methoxy-2-oxo-1,2-dihydro-[1,6]naphthyridine-3-carboxylic acid [5-(3-amino-1-thiophen-3-yl-propylcarbamoyl)-2-methyl-phenyl]-amide

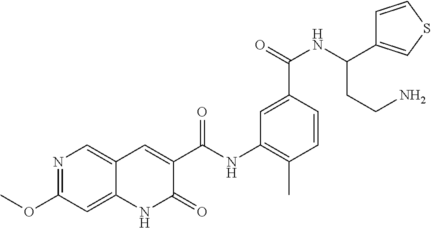

7-Methoxy-2-oxo-1,2-dihydro-[1,6]naphthyridine-3-carboxylic acid [5-(3-amino-1-thiophen-3-yl-propylcarbamoyl)-2-methyl-phenyl]-amide was prepared from (3-{3-[(7-methoxy-2-oxo-1,2-dihydro-[1,6]naphthyridine-3-carbonyl)-amino]-4-methyl-benzoylamino}-3-thiophen-3-yl-propyl)-carbamic acid tert-butyl ester (from Example 62 supra) (100 mg, 0.17 mmol) by following the method in Example 45. The crude product obtained was purified by prep-HPLC (Instrument: Gilson 281, Column. Gemini 5u C18 250×21.5 mm, Mobile Phase: CH₃CN/H₂O (0.1% TFA), 28/72 to 90/10, flow rate 20 mL/min). The resulting solution of product was evaporated, suspended in saturated aqueous NaHCO₃ (10 mL) and stirred for 30 minutes. The solid was collected by filtration and dried to give 7-methoxy-2-oxo-1,2-dihydro-[1,6]naphthyridine-3-carboxylic acid [5-(3-amino-1-thiophen-3-yl-propylcarbamoyl)-2-methyl-phenyl]-amide. (Yield 10 mg, 12%).

¹H NMR (300 MHz, d₆-DMSO) δ 12.04 (d, 1H, J=16.5 Hz), 9.05-8.78 (m, 2H), 8.77-8.62 (m, 1H), 7.63-7.44 (m, 2H), 7.35 (d, 2H, J=7.9 Hz), 7.17 (t, 1H, J=4.5 Hz), 6.61 (s, 1H), 5.27 (m, 1H), 3.95 (s, 3H), 2.98 (m, 1H), 2.62 (m, 1H), 2.39 (s, 3H), 1.94 (m, 2H). LC-MS: [M+H]⁺ 492.1.

### Example 64

### (3-(3-Chloro-phenyl)-3-{3-[(7-methoxy-2-oxo-1,2-dihydro-[1,6]naphthyridine-3-carbonyl)-amino]-4-methyl-benzoylamino}-propyl)-carbamic acid tert-butyl ester

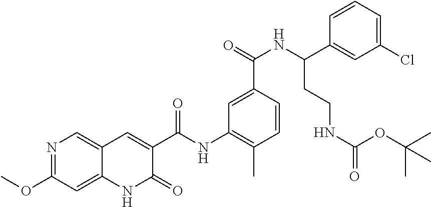

(3-(3-Chloro-phenyl)-3-{3-[(7-methoxy-2-oxo-1,2-dihydro-[1,6]naphthyridine-3-carbonyl)-amino]-4-methyl-benzoylamino}-propyl)-carbamic acid tert-butyl ester was prepared from 3-[(7-methoxy-2-oxo-1,2-dihydro-[1,6]naphthyridine-3-carbonyl)-amino]-4-methyl-benzoic acid (from Example 29 supra) (100 mg, 0.28 mmol) and [3-amino-3-(3-chloro-phenyl)-propyl]-carbamic acid tert-butyl ester (from Example 6 supra) by following the method in Example 32. (Yield 100 mg, 57%). LC-MS: [M-100+1]⁺ 520.

### Example 65

### 7-Methoxy-2-oxo-1,2-dihydro-[1,6]naphthyridine-3-carboxylic acid {5-[3-amino-1-(3-chloro-phenyl)-propylcarbamoyl]-2-methyl-phenyl}-amide; hydrochloride

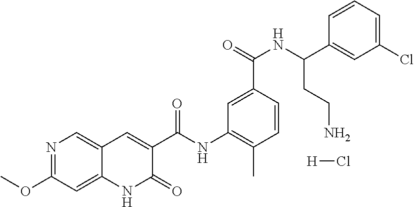

7-Methoxy-2-oxo-1,2-dihydro-[1,6]naphthyridine-3-carboxylic acid {5-[3-amino-1-(3-chloro-phenyl)-propylcarbamoyl]-2-methyl-phenyl}-amide; hydrochloride was prepared from (3-(3-chloro-phenyl)-3-{3-[(7-methoxy-2-oxo-1,2-dihydro-[1,6]naphthyridine-3-carbonyl)-amino]-4-methyl-benzoylamino}-propyl)-carbamic acid tert-butyl ester (from Example 64 supra) (100 mg, 0.16 mmol) by following the method in Example 45. (Yield 30 mg, 31.6%).

¹H NMR (300 MHz, d₆-DMSO) δ 12.64 (s, 1H), 11.78 (s, 1H), 9.11-8.88 (m, 3H), 8.72 (d, 1H, J=1.7 Hz), 8.01 (s, 3H), 7.72-7.50 (m, 2H), 7.46-7.29 (m, 4H), 6.68 (s, 1H), 5.17 (m, 1H), 3.96 (s, 3H), 2.79 (m, 2H), 2.40 (s, 3H), 2.28-2.03 (m, 2H). LC-MS: [M+H]⁺ 518.1.

### Example 66

### [3-{4-Chloro-3-[(7-methoxy-2-oxo-1,2-dihydro-[1,6]naphthyridine-3-carbonyl)-amino]-benzoylamino}-3-(3-chloro-phenyl)-propyl]-carbamic acid tert-butyl ester

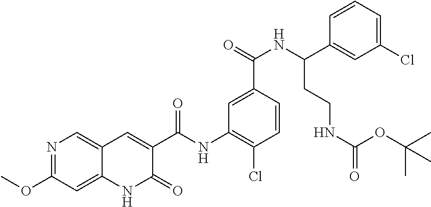

[3-{4-Chloro-3-[(7-methoxy-2-oxo-1,2-dihydro-[1,6]naphthyridine-3-carbonyl)-amino]-benzoylamino}-3-(3-chloro-phenyl)-propyl]-carbamic acid tert-butyl ester was prepared from 4-chloro-3-[(7-methoxy-2-oxo-1,2-dihydro-[1,6]naphthyridine-3-carbonyl)-amino]-benzoic acid (from Example 31 supra) (100 mg, 0.27 mmol) and [3-amino-3-(3-chloro-phenyl)-propyl]-carbamic acid tert-butyl ester (from Example 6 supra) by following the method in Example 32. (Yield 100 mg, 58%).

LC-MS: [M-100+1]⁺ 540.

### Example 67

### 7-Methoxy-2-oxo-1,2-dihydro-[1,6]naphthyridine-3-carboxylic acid {5-[3-amino-1-(3-chloro-phenyl)-propylcarbamoyl]-2-chloro-phenyl}-amide; hydrochloride

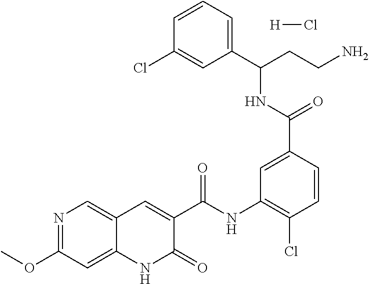

7-Methoxy-2-oxo-1,2-dihydro-[1,6]naphthyridine-3-carboxylic acid {5-[3-amino-1-(3-chloro-phenyl)-propylcarbamoyl]-2-chloro-phenyl}-amide; hydrochloride was prepared from [3-{4-chloro-3-[(7-methoxy-2-oxo-1,2-dihydro-[1,6]naphthyridine-3-carbonyl)-amino]-benzoylamino}-3-(3-chloro-phenyl)-propyl]-carbamic acid tert-butyl ester (from Example 66 supra) (100 mg, 0.16 mmol) by following the method in Example 45. (Yield 60 mg, 62%).

¹H NMR (300 MHz, d₆-DMSO) δ 12.65 (s, 1H), 12.32 (s, 1H), 9.15 (d, 1H, J=8.1 Hz), 9.00 (dd, 3H, J=13.9 Hz, 12.0 Hz), 8.04 (s, 3H), 7.71 (dt, 2H, J=12.7 Hz, 5.1 Hz), 7.54 (s, 1H), 7.48-7.29 (m, 3H), 6.68 (s, 1H), 5.15 (m, 1H), 3.96 (s, 3H), 2.80 (m, 2H), 2.16 (m, 2H). LC-MS: [M+H]⁺ 540.1.

### Example 68

### (3-{4-Chloro-3-[(7-methoxy-2-oxo-1,2-dihydro-[1,6]naphthyridine-3-carbonyl)-amino]-benzoylamino}-3-phenyl-propyl)-carbamic acid tert-butyl ester

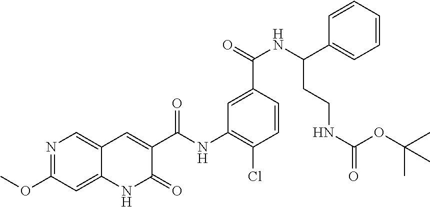

(3-{4-Chloro-3-[(7-methoxy-2-oxo-1,2-dihydro-[1,6]naphthyridine-3-carbonyl)-amino]-benzoylamino}-3-phenyl-propyl)-carbamic acid tert-butyl ester was prepared from 4-chloro-3-[(7-methoxy-2-oxo-1,2-dihydro-[1,6]naphthyridine-3-carbonyl)-amino]-benzoic acid (from Example 31 supra) (100 mg, 0.27 mmol) and (3-amino-3-phenyl-propyl)-carbamic acid tert-butyl ester (from Example 2 supra) by following the method in Example 32. (Yield 120 mg, 74%). LC-MS: [M+H]⁺ 606.

### Example 69

### 7-Methoxy-2-oxo-1,2-dihydro-[1,6]naphthyridine-3-carboxylic acid [5-(3-amino-1-phenyl-propylcarbamoyl)-2-chloro-phenyl]-amide; hydrochloride

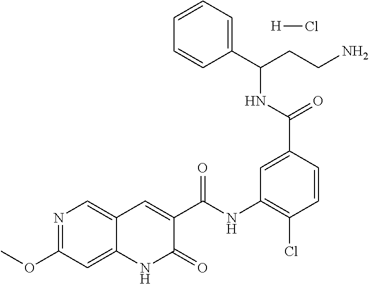

7-Methoxy-2-oxo-1,2-dihydro-[1,6]naphthyridine-3-carboxylic acid [5-(3-amino-1-phenyl-propylcarbamoyl)-2-chloro-phenyl]-amide; hydrochloride was prepared from (3-{4-chloro-3-[(7-methoxy-2-oxo-1,2-dihydro-[1,6]naphthyridine-3-carbonyl)-amino]-benzoylamino}-3-phenyl-propyl)-carbamic acid tert-butyl ester (from Example 68 supra) (120 mg, 0.20 mmol) by following the method in Example 45. (Yield 34 mg, 29.6%).

¹H NMR (300 MHz, d₆-DMSO) δ 12.61 (s, 1H), 12.30 (s, 1H), 9.15-8.87 (m, 4H), 7.91 (s, 3H), 7.68 (s, 2H), 7.36 (m, 5H), 6.66 (s, 1H), 5.16 (m, 1H), 3.96 (d, 3H, J=0.5 Hz), 2.79 (m, 2H), 2.16 (m, 2H). LC-MS: [M+H]⁺ 506.1.

### Example 70

### (2-{4-Chloro-3-[(7-methoxy-2-oxo-1,2-dihydro-[1,6]naphthyridine-3-carbonyl)-amino]-benzoylamino}-2-thiophen-3-yl-ethyl)-carbamic acid tert-butyl ester

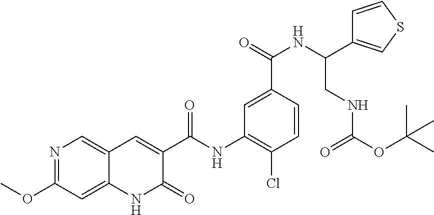

(2-{4-Chloro-3-[(7-methoxy-2-oxo-1,2-dihydro-[1,6]naphthyridine-3-carbonyl)-amino]-benzoylamino}-2-thiophen-3-yl-ethyl)-carbamic acid tert-butyl ester was prepared from 4-chloro-3-[(7-methoxy-2-oxo-1,2-dihydro-[1,6]naphthyridine-3-carbonyl)-amino]-benzoic acid (from Example 31 supra) (100 mg, 0.27 mmol) and (2-amino-2-thiophen-3-yl-ethyl)-carbamic acid tert-butyl ester (from Example 5 supra) by following the method in Example 32. (Yield 100 mg, 62.5%). LC-MS: [M-100+H]⁺ 498.

### Example 71

### 7-Methoxy-2-oxo-1,2-dihydro-[1,6]naphthyridine-3-carboxylic acid [5-(2-amino-1-thiophen-3-yl-ethylcarbamoyl)-2-chloro-phenyl]-amide; hydrochloride

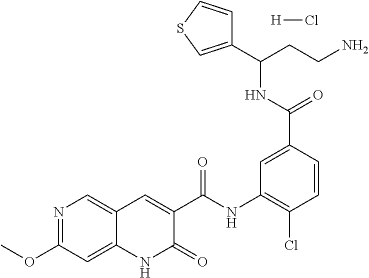

7-Methoxy-2-oxo-1,2-dihydro-[1,6]naphthyridine-3-carboxylic acid [5-(2-amino-1-thiophen-3-yl-ethylcarbamoyl)-2-chloro-phenyl]-amide; hydrochloride was prepared from (2-{4-chloro-3-[(7-methoxy-2-oxo-1,2-dihydro-[1,6]naphthyridine-3-carbonyl)-amino]-benzoylamino}-2-thiophen-3-yl-ethyl)-carbamic acid tert-butyl ester (from Example 70 supra) (100 mg, 0.17 mmol) by following the method in Example 45. (Yield 60 mg, 63%).

¹H NMR (301 MHz, d₆-DMSO+D₂O) δ 9.03 (s, 2H), 8.93 (s, 1H), 7.73 (q, 2H, J=8.5 Hz), 7.55 (dd, 2H, J=15.0 Hz, 10.6 Hz), 7.19 (d, 1H, J=4.9 Hz), 6.66 (s, 1H), 5.47 (t, 1H, J=7.2 Hz), 3.95 (s, 3H), 3.31 (d, J=7.4 Hz, 2H). LC-MS: [M+H]⁺ 498.1.

### Example 72

### 3-{[7-(4-Methyl-piperazin-1-yl)-2-oxo-1,2-dihydro-[1,6]naphthyridine-3-carbonyl]-amino}-benzoic acid methyl ester

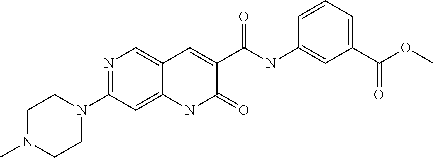

Et₃N (39 mg, 0.382 mmol) was added to a mixture of 7-(4-methyl-piperazin-1-yl)-2-oxo-1,2-dihydro-[1,6]naphthyridine-3-carboxylic acid (100 mg, 0.347 mmol) (from Example 21 supra) and HATU (145 mg, 0.382 mmol) in dry DMF (4 mL) at room temperature. The resultant mixture was stirred until clear solution was obtained. 3-Amino-benzoic acid methyl ester (63 mg, 0.416 mmol) was added. The mixture was stirred for another 20 hours with formation of precipitate. Water (10 mL), aqueous saturated sodium bicarbonate solution (10 mL), and ethyl acetate (25 mL) were added. After thorough stirring, white precipitate was collected by filtration, washed with water and ethyl acetate and dried under reduced pressure to give 3-{[7-(4-methyl-piperazin-1-yl)-2-oxo-1,2-dihydro-[1,6]naphthyridine-3-carbonyl]-amino}-benzoic acid methyl ester. (Yield 88 mg, 60%).

¹H NMR (300 MHz, d₆-DMSO): δ 12.24 (s, 1H), 11.98 (s, 1H), 8.77 (d, 2H, J=10.5 Hz), 8.34 (s, 1H), 7.88 (d, 1H, J=5.1 Hz), 7.68 (d, 1H, J=6.8 Hz), 7.52-7.47 (m, 1H), 6.39 (s, 1H), 3.86 (s, 3H), 3.59 (s, 4H), 2.20 (s, 4H), 1.97 (s, 3H). LC-MS: [M+H]⁺ 421.8.

### Example 73

### 4-Chloro-3-{[7-(4-methyl-piperazin-1-yl)-2-oxo-1,2-dihydro-[1,6]naphthyridine-3-carbonyl]-amino}-benzoic acid methyl ester

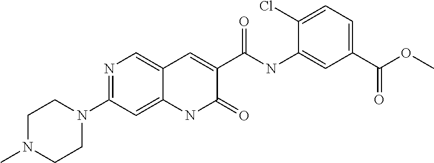

4-Chloro-3-{[7-(4-methyl-piperazin-1-yl)-2-oxo-1,2-dihydro-[1,6]naphthyridine-3-carbonyl]-amino}-benzoic acid methyl ester was prepared from 7-(4-methyl-piperazin-1-yl)-2-oxo-1,2-dihydro-[1,6]naphthyridine-3-carboxylic acid (from Example 21 supra) (50 mg, 0.17 mmol) and 3-amino-4-chloro-benzoic acid methyl ester by following the method in Example 72. (Yield 20 mg, 25%).

¹H NMR (300 MHz, d₆-DMSO): δ 12.49 (s, 1H), 9.21 (s, 1H), 8.86 (s, 1H), 8.79 (s, 1H), 7.70 (s, 2H), 6.42 (s, 1H), 3.89 (s, 3H), 3.63 (s, 4H), 2.42 (s, 4H), 2.23 (s, 3H). LC-MS: [M+H]⁺ 455.8.

### Example 74

### 4-Methyl-3-{[7-(4-methyl-piperazin-1-yl)-2-oxo-1,2-dihydro-[1,6]naphthyridine-3-carbonyl]-amino}-benzoic acid methyl ester

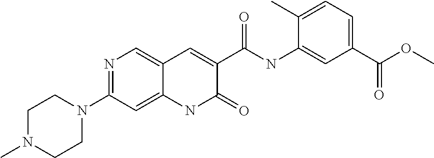

4-Methyl-3-{[7-(4-methyl-piperazin-1-yl)-2-oxo-1,2-dihydro-[1,6]naphthyridine-3-carbonyl]-amino}-benzoic acid methyl ester was prepared from 7-(4-methyl-piperazin-1-yl)-2-oxo-1,2-dihydro-[1,6]naphthyridine-3-carboxylic acid (from Example 21 supra) (66 mg, 0.23 mmol) and 3-amino-4-methyl-benzoic acid methyl ester by following the method in Example 72. (Yield 69 mg, 69%).

¹H NMR (300 MHz, d₆-DMSO): δ 12.23 (s, 1H), 11.94 (s, 1H), 8.98 (s, 1H), 8.85 (s, 1H), 8.79 (s, 1H), 7.65-7.62 (m, 1H), 7.40 (d, 1H, J=8.1 Hz), 6.42 (s, 1H), 3.86 (s, 3H), 3.62 (s, 4H), 2.42 (s, 7H), 2.23 (s, 3H). LC-MS: [M+H]⁺ 435.7.

### Example 75

### (6-Chloro-3-formyl-pyridin-2-yl)-carbamic acid tert-butyl ester

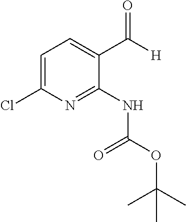

(6-Chloro-3-formyl-pyridin-2-yl)-carbamic acid tert-butyl ester was prepared according to the literature procedure of Siegel, S. et al. WO2009/007029, Jan. 15, 2009.

### Step A

### (6-Chloro-pyridin-2-yl)-carbamic acid tert-butyl ester

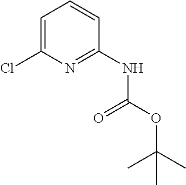

Sodium hexamethyl disilazane (63.03 g, 343 mmol) was added portion-wise to a stirred solution of 2-chloro-6-aminopyridine (20.0 g, 156.25 mmol) in THF (100 mL) at 0° C. under argon. After 5 minutes of stirring at the same temperature, di-tertiary-butyl dicarbonate (36.77 mL, 171 mmol) was added drop-wise into the reaction mixture. After 15 minutes of additional stirring at the same temperature, the reaction temperature was brought to room temperature and stirred until starting material was consumed completely (1 hour, monitored by silica gel TLC using ethyl acetate-hexanes, 1:9 as mobile phase). THF was distilled off under reduced pressure, the obtained residue was taken up in ethyl acetate (300 mL), washed with an aqueous solution of 0.5 M HCl (100 mL), water (2×75 mL), dried over anhydrous sodium sulfate, filtered and concentrated on the rotary evaporator to obtain a gummy mass, which was purified over silica gel column chromatography (eluant:EtOAc-hexanes, 1:19) to give (6-chloro-pyridin-2-yl)-carbamic acid tert-butyl ester as a white solid. (Yield 31.0 g, 87.4%).

### Step B

### (6-Chloro-3-formyl-pyridin-2-yl)-carbamic acid tert-butyl ester

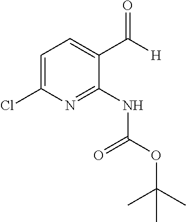

(6-Chloro-pyridin-2-yl)-carbamic acid tert-butyl ester (5.0 g, 21.92 mmol) and 1,2-bis(di-ethylamino)ethane (6.37 g, 54.82 mmol) were dissolved in THF (100 mL) at room temperature under nitrogen and the reaction mixture was cooled to −78° C. To this cooled solution, was added n-butyl lithium (1.8 M in hexanes, 21.92 mL, 54.82 mmol) drop-wise. After completion of addition of n-BuLi, reaction temperature was slowly warmed to −10° C. and held at −10° C. for 2 hours. It was then re-cooled again to −78° C. and DMF (3.39 mL, 43.84 mmol) was added. After completion of addition of DMF, the reaction mixture was again warmed to room temperature slowly and stirring was continued at that temperature until the completion of reaction (1 h, as monitored by silica gel TLC, using ethyl acetate-hexanes 2:8 as mobile phase). Reaction mixture was quenched with aqueous HCl (1N; 30 mL) at −10° C. until the reaction medium was acidic (pH 2-3) and was extracted with ethyl acetate (100 mL). The organic part was separated, washed with water (2×50 mL), brine (50 mL), dried over anhydrous sodium sulfate, filtered and concentrated on the rotary evaporator to give (6-chloro-3-formyl-pyridin-2-yl)-carbamic acid tert-butyl ester as a solid. The obtained product is pure enough to proceed to the next reaction without further purification. (Yield 6.0 g, 99%).

### Example 76

### 2-Amino-6-methoxy-pyridine-3-carbaldehyde

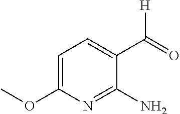

### Step A

### (3-Formyl-6-methoxy-pyridin-2-yl)-carbamic acid tert-butyl ester

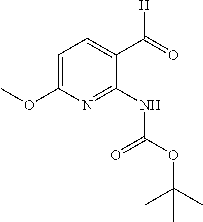

To a stirred solution of (6-chloro-3-formyl-pyridin-2-yl)-carbamic acid tert-butyl ester (25.2 g, 98.43 mmol) (from Example 75 supra) in methanol (504 mL) was added sodium methoxide (8.505 g, 157.5 mmol) at room temperature under nitrogen and the reaction mixture was stirred overnight at the same temperature. Silica gel TLC analysis confirmed the completion of reaction (mobile phase:ethyl acetate-hexanes 3:7). Methanol was distilled off under reduced pressure and the crude organic mixture was taken up in EtOAc (500 mL). The organic part was washed with water (2×100 mL), dried over sodium sulfate, filtered and concentrated under reduced pressure to give crude product, which was purified over silica gel column chromatography (eluant:EtOAc-Hexanes 1:19) to afford (3-formyl-6-methoxy-pyridin-2-yl)-carbamic acid tert-butyl ester as a yellow solid. (Yield 16.68 g, 67.2%).

LC-MS (ES⁺) [M+H]⁺ 253.

### Step B

### 2-Amino-6-methoxy-pyridine-3-carbaldehyde

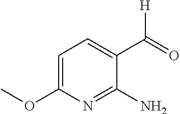

A solution of HCl in dioxane (3.5 M solution; 600 mL) was added to (3-formyl-6-methoxy-pyridin-2-yl)-carbamic acid tert-butyl ester (20 g, 79.36 mmol) at room temperature under nitrogen and the solution was stirred overnight at the same temperature. Silica gel TLC analysis confirmed the completion of reaction (mobile phase:ethyl acetate-hexanes 2:8). Reaction mixture was concentrated under reduced pressure and the obtained gummy mass was triturated with diethyl ether to obtain crude 2-amino-6-methoxy-pyridine-3-carbaldehyde as an off-white solid. (Yield 14.13 g). LC-MS (ES⁺) [M+H]⁺ 153.

### Example 77

### 7-Methoxy-2-oxo-1,2-dihydro-1,8-naphthyridine-3-carboxylic acid methyl ester

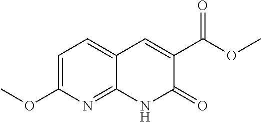

Dimethyl malonate (21.5 mL, 188.28 mmol), piperidine (19.3 mL, 195.24 mmol) and acetic acid (0.40 mL, 6.97 mmol) were added simultaneously to a stirred solution of crude 2-amino-6-methoxy-pyridine-3-carbaldehyde (10.6 g, 69.73 mmol) (from Example 76 supra) in methanol (328 mL) at room temperature and the reaction mixture was heated at 60° C. for 18 hours. Silica gel TLC analysis confirmed the completion of reaction (mobile phase: methanol-dichloromethane 1:9). Reaction temperature was cooled to room temperature and solvents were distilled off under reduced pressure to obtain a crude 7-methoxy-2-oxo-1,2-dihydro-[1,8]naphthyridine-3-carboxylic acid methyl ester, which was directly used for the next step reaction without further purification. (Yield 21 g, crude).

LC-MS (ES⁺) [M+H]⁺ 235.

### Example 78

### 7-Methoxy-2-oxo-1,2-dihydro-[1,8]naphthyridine-3-carboxylic acid

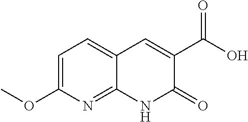

A mixture of crude 7-methoxy-2-oxo-1,2-dihydro-[1,8]naphthyridine-3-carboxylic acid methyl ester (21 g, 89.74 mmol) (from Example 77 supra) and sodium hydroxide (10.76 g, 269.23 mmol) in methanol-water mixture (1:1; 370 mL) was heated at 65° C. for 18 hours. After cooling to room temperature, the reaction mixture was concentrated under reduced pressure. The resulting material was acidified (pH ˜2-3) with the drop-wise addition of an aqueous solution of 2N HCl, during which time a white precipitate was formed and was collected by filtration. The collected material was washed with water and dried in air to give 7-methoxy-2-oxo-1,2-dihydro-[1,8]naphthyridine-3-carboxylic acid as a pale yellow solid. (Yield 9.2 g, 46.6%).

LC-MS (ES⁺) [M+H]⁺ 221.

### Example 79

### 2-Amino-6-(4-methyl-piperazin-1-yl)-pyridine-3-carbaldehyde

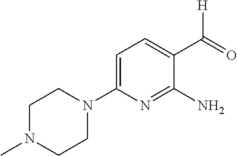

### Step A

### [3-Formyl-6-(4-methyl-piperazin-1-yl)-pyridin-2-yl]-carbamic acid tert-butyl ester

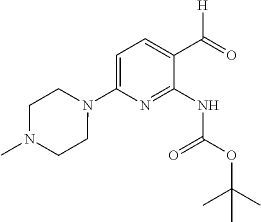

To a stirred solution of (6-chloro-3-formyl-pyridin-2-yl)-carbamic acid tert-butyl ester (6.0 g, 23.43 mmol) (from Example 75 supra) in DMSO (100 mL) were added 1-methylpiperizine (3.05 g, 30.40 mmol) and potassium carbonate (6.47 g, 46.8 mmol) at room temperature and the reaction mixture was stirred at 80° C. for 3 hours. (Silica gel TLC analysis using MeOH-dichloromethane 1:9). Reaction mixture was quenched with water (500 mL) and was extracted with dichloromethane (3×100 mL). Combined organic parts were washed with water (100 mL), brine (100 mL), dried over sodium sulfate and evaporated under reduced pressure to give [3-formyl-6-(4-methyl-piperazin-1-yl)-pyridin-2-yl]-carbamic acid tert-butyl ester as a yellow solid. (Yield 4.0 g, 53.3%).

LC-MS (ES⁺) [M+H]⁺ 321.

### Step B

### 2-Amino-6-(4-methyl-piperazin-1-yl)-pyridine-3-carbaldehyde

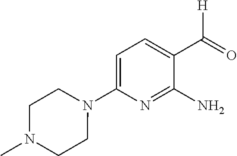

A solution of HCl in dioxane (3.5 M solution; 400 mL) was added to [3-formyl-6-(4-methyl-piperazin-1-yl)-pyridin-2-yl]-carbamic acid tert-butyl ester (10 g, 31.0 mmol) at room temperature under nitrogen and stirred overnight at the same temperature. Silica gel TLC analysis confirmed the completion of reaction (MeOH-dichloromethane 1:9). Reaction mixture was concentrated under reduced pressure to give crude 2-amino-6-(4-methyl-piperazin-1-yl)-pyridine-3-carbaldehyde. (Yield 9.2 g). LC-MS (ES⁺) [M+H]⁺ 221.

### Example 80

### 7-(4-Methyl-piperazin-1-yl)-2-oxo-1,2-dihydro-[1,8]naphthyridine-3-carboxylic acid methyl ester

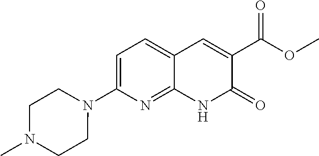

Dimethyl malonate (9.93 mL, 86.97 mmol), piperidine (9.31 mL, 94.08 mmol) and acetic acid (0.20 mL, 3.55 mmol) were added simultaneously to a stirred solution of crude 2-amino-6-(4-methyl-piperazin-1-yl)-pyridine-3-carbaldehyde (6.9 g, 31.36 mmol) (from Example 79 supra) in methanol (100 mL) at room temperature and the reaction mixture was heated at 60° C. for 18 hours. Silica gel TLC analysis confirmed the completion of reaction (MeOH-dichloromethane 1:9). Mixture was cooled to room temperature and solvents were distilled off under reduced pressure to obtain crude 7-(4-methyl-piperazin-1-yl)-2-oxo-1,2-dihydro-[1,8]naphthyridine-3-carboxylic acid methyl ester which was directly used in the next step without further purification. (Yield 11.0 g). LC-MS (ES⁺) [M+H]⁺ 303.

### Example 81

### 7-(4-Methyl-piperazin-1-yl)-2-oxo-1,2-dihydro-[1,8]naphthyridine-3-carboxylic acid; hydrochloride

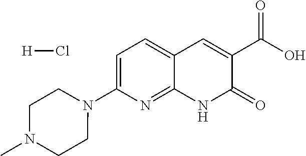

A mixture of crude 7-(4-methyl-piperazin-1-yl)-2-oxo-1,2-dihydro-[1,8]naphthyridine-3-carboxylic acid methyl ester (22.0 g, 72.8 mmol) (from Example 80 supra) and sodium hydroxide (8.741 g, 218.54 mmol) in methanol-water mixture (1:1; 220 mL) was heated at 65° C. for 18 hours. Reaction went to completion during the time (silica gel TLC analysis, MeOH-dichloromethane 1:9). After cooling to room temperature, the reaction mixture was concentrated under reduced pressure, the obtained material was diluted with water (100 mL) and was extracted with dichloromethane (2×100 mL) [to get rid of unwanted organic impurities]. Organic part was discarded and the aqueous layer was acidified with an aqueous solution of HCl (2N, to pH ˜2-3), during which a white precipitate was formed, which was collected by filtration. The residue was washed with water and dried in air to give 7-(4-methyl-piperazin-1-yl)-2-oxo-1,2-dihydro-[1,8]naphthyridine-3-carboxylic acid; hydrochloride as a yellow solid. (Yield 10.0 g, 47.8%). LC-MS (ES⁺) [M+H]⁺ 289.

### Example 82

### 2-Amino-6-chloro-pyridine-3-carbaldehyde

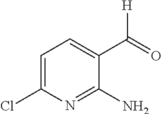

2-Amino-6-chloro-pyridine-3-carbaldehyde was prepared from (6-chloro-3-formyl-pyridin-2-yl)-carbamic acid tert-butyl ester (4.0 g, 15.62 mmol) (from Example 75 supra) in 4 M solution of HCl in dioxane (108 mL, 437 mmol) by following method in Example 76 as an off-white solid. (Yield 3 g, quantitative). LC-MS [M+H]⁺ 157.

### Example 83

### 7-Chloro-2-oxo-1,2-dihydro-[1,8]naphthyridine-3-carboxylic acid methyl ester

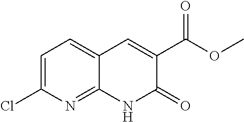

To a cold (0° C.) solution of 2-amino-6-chloro-pyridine-3-carbaldehyde was prepared from (6-chloro-3-formyl-pyridin-2-yl)-carbamic acid tert-butyl ester (3.0 g, 19.23 mmol) (from Example 82 supra) in dichloromethane (90 mL) were added dry pyridine (4.66 mL, 57.69 mmol) and methyl malonyl chloride (6.66 mL, 61.53 mmol) at room temperature under nitrogen and stirred overnight at the same temperature. Silica gel TLC analysis confirmed the completion of reaction. The reaction mixture was quenched with the addition of an aqueous solution of saturated NaHCO₃ (50 mL) and was extracted with dichloromethane (3×50 mL). Collected organic parts were dried over sodium sulfate, filtered and concentrated under reduced pressure to give a crude material, which was purified by column chromatography, using ethyl acetate-hexanes 2:3 as eluent, to give 7-chloro-2-oxo-1,2-dihydro-[1,8]naphthyridine-3-carboxylic acid methyl ester as a pale yellow solid. (Yield 2.9 g, 63.4%).

LC-MS [M+H]⁺ 239.

### Example 84

### 7-[(2,2-Dimethyl-[1,3]dioxolan-4-ylmethyl)-amino]-2-oxo-1,2-dihydro-[1,8]naphthyridine-3-carboxylic acid methyl ester

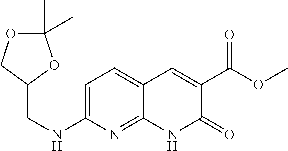

To a stirred solution of 7-chloro-2-oxo-1,2-dihydro-[1,8]naphthyridine-3-carboxylic acid methyl ester (3.0 g, 12.6 mmol) (from Example 83 supra) in DMSO (60 mL) was added triethylamine (3.49 mL, 25.2 mmol) and (2,2-dimethyl-[1,3]dioxolan-4-yl)-methylamine (2.15 g, 16.38 mmol) at room temperature and reaction mixture was heated at 140° C. until starting material was consumed completely (2 hour; as monitored by silica gel TLC analysis). Reaction mixture was cooled to room temperature, poured into cold water and was extracted with dichloromethane (3×50 mL). Separated organic parts were combined, dried over sodium sulfate, filtered and concentrated to give a crude gummy mass, which was purified by column chromatography, using methanol-dichloromethane 1:49 as eluent, to afford 7-[(2,2-dimethyl-[1,3]dioxolan-4-ylmethyl)-amino]-2-oxo-1,2-dihydro-[1,8]naphthyridine-3-carboxylic acid methyl ester as a pale yellow solid. (Yield 2.3 g, 54.8%).

LC-MS (ES⁺) [M+H]⁺ 334.

### Example 85

### 7-(2,3-Dihydroxy-propylamino)-2-oxo-1,2-dihydro-[1,8]naphthyridine-3-carboxylic acid

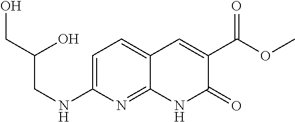

7-(2,3-Dihydroxy-propylamino)-2-oxo-1,2-dihydro-[1,8]naphthyridine-3-carboxylic acid was prepared from 7-[(2,2-dimethyl-[1,3]dioxolan-4-ylmethyl)-amino]-2-oxo-1,2-dihydro-[1,8]naphthyridine-3-carboxylic acid methyl ester (1.0 g, 3 mmol) (from Example 84 supra) and sodium hydroxide (0.36 g, 9 mmol) in methanol-water mixture (1:1, 25 mL) by the method in Example 78 as a white solid. (Yield 0.67 g, 79.96%). LC-MS (ES⁺) [M+H]⁺ 280.

### Example 86

### 3-{[7-(4-Methyl-piperazin-1-yl)-2-oxo-1,2-dihydro-[1,8]naphthyridine-3-carbonyl]-amino}-benzoic acid methyl ester

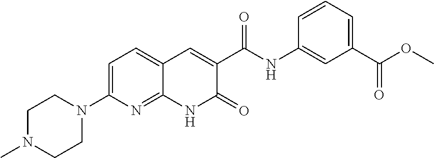

To a stirred solution of 7-(4-methyl-piperazin-1-yl)-2-oxo-1,2-dihydro-[1,8]naphthyridine-3-carboxylic acid; hydrochloride (0.15 g, 0.46 mmol) (from Example 81 supra) and HATU (0.238 g, 0.62 mmol) in dry DMF (8 mL) was added triethylamine (0.2 mL, 1.3 mmol) at room temperature. After 15 minutes stirring at the same temperature, 3-amino-benzoic acid methyl ester (0.0945 g, 0.62 mmol) was added to this mixture. Stirring was continued for another 20 hours, during which time the reaction went for completion with the formation of precipitate. Water (50 mL), aqueous solution of saturated sodium bicarbonate (10 mL) and ethyl acetate (25 mL) were added. After thorough mixing, white precipitate was collected by filtration, washed with water and ethyl acetate and dried under reduced pressure to give 3-{[7-(4-Methyl-piperazin-1-yl)-2-oxo-1,2-dihydro-[1,8]naphthyridine-3-carbonyl]-amino}-benzoic acid methyl ester. (Yield 0.20 g, 91.2%). LC-MS (ES⁺) [M+H]⁺ 422.

### Example 87

### 4-Methyl-3-{[7-(4-methyl-piperazin-1-yl)-2-oxo-1,2-dihydro-[1,8]naphthyridine-3-carbonyl]-amino}-benzoic acid methyl ester

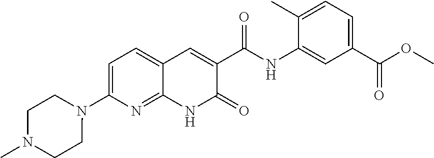

4-Methyl-3-{[7-(4-methyl-piperazin-1-yl)-2-oxo-1,2-dihydro-[1,8]naphthyridine-3-carbonyl]-amino}-benzoic acid methyl ester was prepared by the method in Example 86 from 7-(4-methyl-piperazin-1-yl)-2-oxo-1,2-dihydro-[1,8]naphthyridine-3-carboxylic acid; hydrochloride (0.1 g, 0.3 mmol) (from Example 81 supra), 3-amino-4-methyl-benzoic acid methyl ester (0.071 g, 0.43 mmol), HATU (0.163 g, 0.43 mmol) and triethylamine (0.12 mL, 0.89 mmol) in dry DMF (5 mL). (Yield 0.06 g, 40%).

LC-MS (ES⁺) [M+H]⁺ 436.

### Example 88

### 4-Chloro-3-{[7-(4-methyl-piperazin-1-yl)-2-oxo-1,2-dihydro-[1,8]naphthyridine-3-carbonyl]-amino}-benzoic acid methyl ester

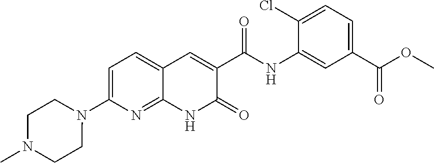

4-Chloro-3-{[7-(4-methyl-piperazin-1-yl)-2-oxo-1,2-dihydro-[1,8]naphthyridine-3-carbonyl]-amino}-benzoic acid methyl ester was prepared by the method in Example 86 from 7-(4-methyl-piperazin-1-yl)-2-oxo-1,2-dihydro-[1,8]naphthyridine-3-carboxylic acid; hydrochloride (0.1 g, 0.3 mmol) (from Example 81 supra), 3-amino-4-chloro-benzoic acid methyl ester (0.079 g, 0.43 mmol), HATU (0.163 g, 0.43 mmol) and triethylamine (0.12 mL, 0.89 mmol) in dry DMF (5 mL). (Yield 0.070 g, 44.3%).

LC-MS (ES⁺) [M+H]⁺ 456.

### Example 89

### 3-[(7-Methoxy-2-oxo-1,2-dihydro-[1,8]naphthyridine-3-carbonyl)-amino]-4-methyl-benzoic acid methyl ester

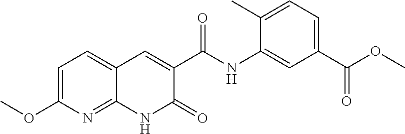

3-[(7-Methoxy-2-oxo-1,2-dihydro-[1,8]naphthyridine-3-carbonyl)-amino]-4-methyl-benzoic acid methyl ester was prepared by the method in Example 86 from 7-methoxy-2-oxo-1,2-dihydro-[1,8]naphthyridine-3-carboxylic acid (0.2 g, 0.91 mmol) (from Example 78 supra), 3-amino-4-methyl-benzoic acid methyl ester (0.18 g, 1.09 mmol), HATU (0.0.415 g, 1.09 mmol) and triethylamine (0.3 mL, 2.27 mmol) in dry DMF (8 mL). (Yield 0.203 g, 60.8%).

LC-MS (ES⁺) [M+H]⁺ 368.

### Example 90

### 4-Chloro-3-[(7-methoxy-2-oxo-1,2-dihydro-[1,8]naphthyridine-3-carbonyl)-amino]-benzoic acid methyl ester

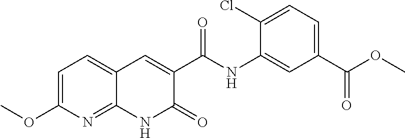

4-Chloro-3-[(7-methoxy-2-oxo-1,2-dihydro-[1,8]naphthyridine-3-carbonyl)-amino]-benzoic acid methyl ester was prepared by the method in Example 86 from 7-methoxy-2-oxo-1,2-dihydro-[1,8]naphthyridine-3-carboxylic acid (0.2 g, 0.91 mmol) (from Example 78 supra), 3-amino-4-chloro-benzoic acid methyl ester (0.202 g, 1.09 mmol), HATU (0.0.415 g, 1.09 mmol) and triethylamine (0.3 mL, 2.27 mmol) in dry DMF (8 mL). (Yield 0.127 g, 36.1%).

LC-MS (ES⁺) [M+H]⁺ 388.

### Example 91

### 3-[(7-Methoxy-2-oxo-1,2-dihydro-[1,6]naphthyridine-3-carbonyl)-amino]-benzoic acid methyl ester

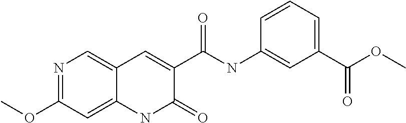

3-[(7-Methoxy-2-oxo-1,2-dihydro-[1,6]naphthyridine-3-carbonyl)-amino]-benzoic acid methyl ester was prepared from 7-methoxy-2-oxo-1,2-dihydro-[1,6]naphthyridine-3-carboxylic acid (from Example 15 supra) (44 mg, 0.21 mmol) and 3-amino-benzoic acid methyl ester by following the method in Example 30. (Yield 30 mg, 42%).

¹H NMR (300 MHz, d₆-DMSO) δ 11.94 (s, 1H), 8.97 (s, 1H), 8.91 (s, 1H), 8.38 (s, 1H), 7.92 (d, 1H, J=9.3 Hz), 7.72 (d, 1H, J=7.8 Hz), 7.55-7.51 (m, 1H), 6.62 (s, 1H), 3.95 (s, 3H), 3.88 (s, 3H). LC-MS: [M+H]⁺ 354.

### Example 92

### 4-Chloro-3-(7-methoxy-2-oxo-1,2-dihydro-1,8-naphthyridine-3-carboxamido)benzoic acid

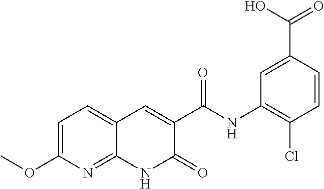

### Step A

### Methyl 4-chloro-3-(7-methoxy-2-oxo-1,2-dihydro-1,8-naphthyridine-3-carboxamido)benzoate

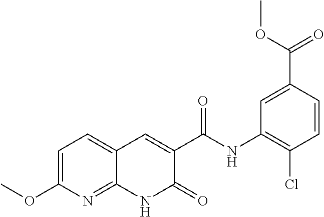

Triethylamine (0.71 mL, 5.09 mmol) was added to a mixture of 7-methoxy-2-oxo-1,2-dihydro-1,8-naphthyridine-3-carboxylic acid (from Example 78 supra) (1.0 g, 4.54 mmol) and HATU (1.93 g, 5.09 mmol) in DMF (10 mL). Mixture was stirred at room temperature for 30 minutes. Methyl 3-amino-4-chlorobenzoate (1.01 g, 5.45 mmol) was added. Mixture was stirred at room temperature for 18 hours and then partitioned between ethyl acetate and water. The precipitate was collected by filtration, washed with water and ethyl acetate and dried in vacuum oven to give methyl 4-chloro-3-(7-methoxy-2-oxo-1,2-dihydro-1,8-naphthyridine-3-carboxamido)benzoate. (Yield 1.4 g, 79.5%).

LR-MS: [M+H]⁺ 388.

### Step B

### 4-Chloro-3-(7-methoxy-2-oxo-1,2-dihydro-1,8-naphthyridine-3-carboxamido)benzoic acid

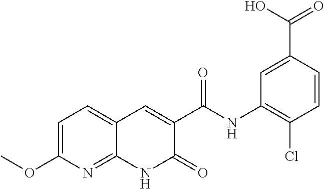

A solution of methyl 4-chloro-3-(7-methoxy-2-oxo-1,2-dihydro-1,8-naphthyridine-3-carboxamido)benzoate (1.38 g, 3.56 mmol) in methanol (100 mL) was added 2N NaOH solution (15.0 mL). Mixture was heated at reflux for 18 hours. After cooling, mixture was concentrated under reduced pressure to remove most of the methanol. The residue was diluted with water (20 mL) and acidified with 1N hydrochloric acid solution to pH 2. Brown precipitate formed was collected by filtration and washed with water and dried in vacuum oven to give 4-chloro-3-(7-methoxy-2-oxo-1,2-dihydro-1,8-naphthyridine-3-carboxamido) benzoic acid as brown powder. (Yield 1.21 g, 91.0%).

### Example 93

### 3-(7-Methoxy-2-oxo-1,2-dihydro-1,8-naphthyridine-3-carboxamido)-4-methylbenzoic acid

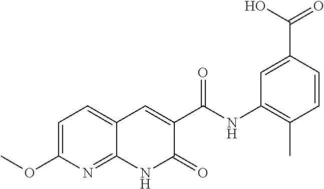

### Step A

### Methyl 3-(7-methoxy-2-oxo-1,2-dihydro-1,8-naphthyridine-3-carboxamido)-4-methylbenzoate

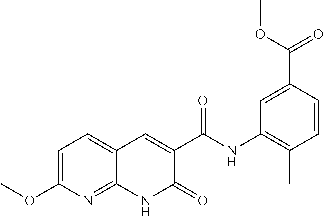

Triethylamine (0.71 mL, 5.09 mmol) was added to a mixture of 7-methoxy-2-oxo-1,2-dihydro-1,8-naphthyridine-3-carboxylic acid (from Example 78 supra) (1.0 g, 4.54 mmol) and HATU (1.93 g, 5.09 mmol) in DMF (10 mL). The mixture was stirred at room temperature for 30 minutes. Methyl 3-amino-4-methylbenzoate (0.90 g, 5.45 mmol) was added. The mixture was stirred at room temperature for 18 hours and then partitioned between ethyl acetate and water. Precipitate was collected by filtration, washed with water and ethyl acetate and dried in vacuum oven to give methyl 3-(7-methoxy-2-oxo-1,2-dihydro-1,8-naphthyridine-3-carboxamido)-4-methylbenzoate. (Yield 1.36 g, 81.5%).

### Step B

### 3-(7-Methoxy-2-oxo-1,2-dihydro-1,8-naphthyridine-3-carboxamido)-4-methylbenzoic acid

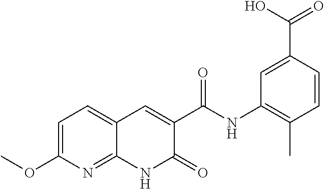

A solution of methyl 3-(7-methoxy-2-oxo-1,2-dihydro-1,8-naphthyridine-3-carboxamido)-4-methylbenzoate (1.33 g, 3.62 mmol) in methanol (100 mL) was added 2N NaOH solution (15.0 ml). Mixture was heated at 50° C. for 18 hours. After cooling, the mixture was concentrated under reduced pressure to remove most of the methanol. The residue was diluted with water (20 mL) and acidified with 1N hydrochloric acid solution to pH 2. Yellow precipitate formed was collected by filtration and washed with water and dried in vacuum oven to give 3-(7-methoxy-2-oxo-1,2-dihydro-1,8-naphthyridine-3-carboxamido)-4-methylbenzoic acid. (Yield 1.22 g, 95.4%).

HR-MS (ES⁺) m/z Calculated for C₁₈H₁₆N₃O₅ ([M+H]⁺): 354.1085. Found: 354.1085.

### Example 94

### N-(5-(Benzylcarbamoyl)-2-chlorophenyl)-7-methoxy-2-oxo-1,2-dihydro-1,8-naphthyridine-3-carboxamide

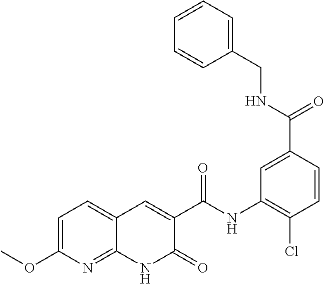

Triethylamine (0.021 ml, 0.15 mmol) was added to a mixture of 4-chloro-3-(7-methoxy-2-oxo-1,2-dihydro-1,8-naphthyridine-3-carboxamido)benzoic acid (from Example 92 supra) (0.05 g, 0.13 mmol) and HATU (0.057 g, 0.15 mmol) in DMF (2 mL). Mixture was stirred at room temperature for 30 minutes. Benzylamine (0.017 g, 0.16 mmol) was added. Mixture was stirred at room temperature for 18 hours and then partitioned between ethyl acetate and water. The aqueous phase was extracted with ethyl acetate (3×). The combined organic phase was washed with water and brine, dried (magnesium sulfate) and concentrated. The residue was collected by filtration, washed with ethyl acetate and water and dried in vacuum oven to give N-(5-(benzylcarbamoyl)-2-chlorophenyl)-7-methoxy-2-oxo-1,2-dihydro-1,8-naphthyridine-3-carboxamide. (Yield 0.051 g, 82.4%).

HR-MS (ES⁺) m/z Calculated for C₂₄H₁₉ClN₄NaO₄ ([M+Na]⁺): 485.0987. Found: 485.0988.

### Example 95

### N-(2-Chloro-5-(3-chlorobenzylcarbamoyl)phenyl)-7-methoxy-2-oxo-1,2-dihydro-1,8-naphthyridine-3-carboxamide

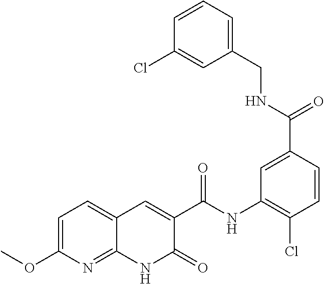

Triethylamine (0.021 mL, 0.15 mmol) was added to a mixture of 4-chloro-3-(7-methoxy-2-oxo-1,2-dihydro-1,8-naphthyridine-3-carboxamido)benzoic acid (from Example 92 supra) (0.05 g, 0.13 mmol) and HATU (0.057 g, 0.15 mmol) in DMF (2 mL). Mixture was stirred at room temperature for 30 minutes. 3-Chlorobenzylamine (0.023 g, 0.16 mmol) was added. Mixture was stirred at room temperature for 18 hours and then partitioned between ethyl acetate and water. The aqueous phase was extracted with ethyl acetate (3×). The combined organic phase was washed with water and brine, dried (magnesium sulfate) and concentrated. The residue was collected by filtration, washed with ethyl acetate and water and dried in vacuum oven to give N-(2-chloro-5-(3-chlorobenzylcarbamoyl)phenyl)-7-methoxy-2-oxo-1,2-dihydro-1,8-naphthyridine-3-carboxamide. (Yield 0.052 g, 78.2%).

HR-MS (ES⁺) m/z Calculated for C₂₄H₁₈Cl₂N₄NaO₄ ([M+Na]⁺): 519.0597. Found: 519.0597.

### Example 96

### tert-Butyl 3-(4-chloro-3-(7-methoxy-2-oxo-1,2-dihydro-1,8-naphthyridine-3-carboxamido)benzamido)-3-phenylpropylcarbamate

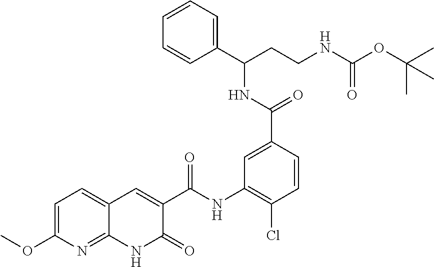

Triethylamine (0.042 mL, 0.30 mmol) was added to a mixture of 4-chloro-3-(7-methoxy-2-oxo-1,2-dihydro-1,8-naphthyridine-3-carboxamido)benzoic acid (from Example 92 supra) (0.10 g, 0.27 mmol) and HATU (0.11 g, 0.30 mmol) in DMF (4 mL). Mixture was stirred at room temperature for 30 minutes. (3-Amino-3-phenyl-propyl)-carbamic acid tert-butyl ester (from Example 2 supra) (0.080 g, 0.32 mmol) was added. Mixture was stirred for 18 hours and then partitioned between ethyl acetate and water. The aqueous phase was extracted with ethyl acetate (3×). The combined organic phase was washed with water and brine, dried (magnesium sulfate) and concentrated. The residue was collected by filtration, washed with ethyl acetate and water and dried in vacuum oven to give tert-butyl 3-(4-chloro-3-(7-methoxy-2-oxo-1,2-dihydro-1,8-naphthyridine-3-carboxamido) benzamido)-3-phenylpropylcarbamate. (Yield 0.11 g, 67.8%).

HR-MS (ES⁺) m/z Calculated for C₃₁H₃₂ClN₅NaO₆ ([M+Na]⁺): 628.1933. Found: 628.1955.

### Example 97

### tert-Butyl 3-(3-(7-methoxy-2-oxo-1,2-dihydro-1,8-naphthyridine-3-carboxamido)-4-methylbenzamido)-3-phenylpropylcarbamate

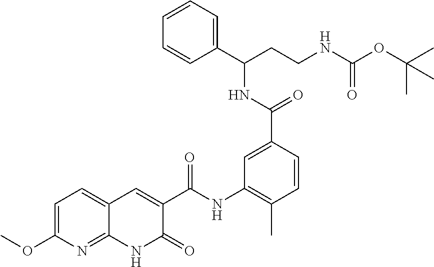

Triethylamine (0.044 mL, 0.32 mmol) was added to a mixture of 3-(7-methoxy-2-oxo-1,2-dihydro-1,8-naphthyridine-3-carboxamido)-4-methylbenzoic acid (from Example 93 supra) (0.10 g, 0.28 mmol) and HATU (0.12 g, 0.32 mmol) in DMF (4 mL). The resultant mixture was stirred at room temperature for 30 minutes. (3-Amino-3-phenyl-propyl)-carbamic acid tert-butyl ester (from Example 2 supra) (0.085 g, 0.34 mmol) was added. The mixture was stirred at room temperature for 18 hours and then partitioned between ethyl acetate and water. The aqueous phase was extracted with ethyl acetate (3×). The combined organic phase was washed with water and brine, dried (magnesium sulfate) and concentrated. The residue was collected by filtration, washed with ethyl acetate and water and dried in vacuum oven to give tert-butyl 3-(3-(7-methoxy-2-oxo-1,2-dihydro-1,8-naphthyridine-3-carboxamido)-4-methylbenzamido)-3-phenylpropylcarbamate. (Yield 0.089 g, 53.7%).

HR-MS (ES⁺) m/z Calculated for C₃₂H₃₅N₅NaO₆ ([M+Na]⁺): 608.2479. Found: 608.2477.

### Example 98

### N-(5-(Benzylcarbamoyl)-2-methylphenyl)-7-methoxy-2-oxo-1,2-dihydro-1,8-naphthyridine-3-carboxamide

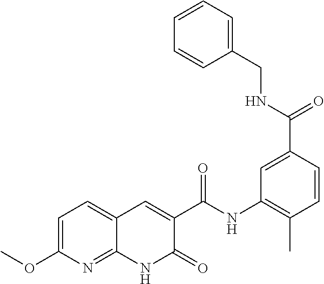

Triethylamine (0.022 mL, 0.16 mmol) was added to a mixture of 3-(7-methoxy-2-oxo-1,2-dihydro-1,8-naphthyridine-3-carboxamido)-4-methylbenzoic acid (from Example 93 supra) (0.05 g, 0.14 mmol) and HATU (0.06 g, 0.16 mmol) in DMF (2 mL). Mixture was stirred at room temperature for 30 minutes. Benzylamine (0.018 g, 0.17 mmol) was added. Mixture was stirred at room temperature for 18 hours and then partitioned between ethyl acetate and water. The aqueous phase was extracted with ethyl acetate (3×). The combined organic phase was washed with water and brine, dried (magnesium sulfate) and concentrated. The residue was collected by filtration, washed with ethyl acetate and water and dried in vacuum oven to give N-(5-(benzylcarbamoyl)-2-methylphenyl)-7-methoxy-2-oxo-1,2-dihydro-1,8-naphthyridine-3-carboxamide. (Yield 0.048 g, 76.7%).

HR-MS (ES⁺) m/z Calculated for C₂₅H₂₂N₄NaO₄ ([M+Na]⁺): 465.1533. Found: 465.1534.

### Example 99

### N-(5-(3-Chlorobenzylcarbamoyl)-2-methylphenyl)-7-methoxy-2-oxo-1,2-dihydro-1,8-naphthyridine-3-carboxamide

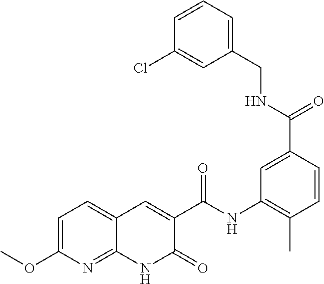

Triethylamine (0.022 mL, 0.16 mmol) was added to a mixture of 3-(7-methoxy-2-oxo-1,2-dihydro-1,8-naphthyridine-3-carboxamido)-4-methylbenzoic acid (from Example 93 supra) (0.05 g, 0.14 mmol) and HATU (0.06 g, 0.16 mmol) in DMF (2 mL). The resultant mixture was stirred at room temperature for 30 minutes. 3-Chlorobenzylamine (0.024 g, 0.17 mmol) was added. The mixture was stirred at room temperature for 18 hours and then partitioned between ethyl acetate and water. The aqueous phase was extracted with ethyl acetate (3×). The combined organic phase was washed with water and brine, dried (magnesium sulfate) and concentrated. The residue was collected by filtration, washed with ethyl acetate and water and dried in vacuum oven to give N-(5-(3-chlorobenzylcarbamoyl)-2-methylphenyl)-7-methoxy-2-oxo-1,2-dihydro-1,8-naphthyridine-3-carboxamide. (Yield 0.052 g, 77.1%).

HR-MS (ES⁺) m/z Calculated for C₂₅H₂₁ClN₄NaO₄ ([M+Na]⁺): 499.1143. Found: 499.1143.

### Example 100

### N-(5-(3-Amino-1-phenylpropylcarbamoyl)-2-methylphenyl)-7-methoxy-2-oxo-1,2-dihydro-1,8-naphthyridine-3-carboxamide

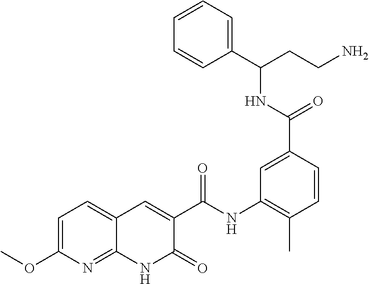

To a solution of tert-butyl 3-(3-(7-methoxy-2-oxo-1,2-dihydro-1,8-naphthyridine-3-carbox-amido)-4-methylbenzamido)-3-phenylpropylcarbamate (from Example 97 supra) (0.079 g, 0.13 mmol) in dichloromethane (5 mL) was added trifluoroacetic acid (1.0 mL). The mixture was stirred at room temperature for 18 hours. The mixture was concentrated and then partitioned between dichoromethane and saturated sodium carbonate solution. The precipitate was collected by filtration and washed with dichloromethane, methanol and water and dried in vacuum oven to give N-(5-(3-amino-1-phenylpropylcarbamoyl)-2-methylphenyl)-7-methoxy-2-oxo-1,2-dihydro-1,8-naphthyridine-3-carboxamide. (Yield 0.045 g, 69.1%).

HR-MS (ES⁺) m/z Calculated for C₂₇H₂₈N₅O₄ ([M+H]⁺): 486.2136. Found: 486.2138.

### Example 101

### N-(5-(3-Amino-1-phenylpropylcarbamoyl)-2-chlorophenyl)-7-methoxy-2-oxo-1,2-dihydro-1,8-naphthyridine-3-carboxamide

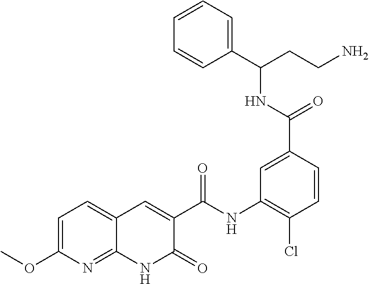

To a solution of tert-butyl 3-(4-chloro-3-(7-methoxy-2-oxo-1,2-dihydro-1,8-naphthyridine-3-carboxamido)benzamido)-3-phenylpropylcarbamate (from Example 96 supra) (0.09 g, 0.15 mmol) in dichloromethane (5 mL) was added trifluoroacetic acid (1.0 mL). The mixture was stirred at room temperature for 18 hours. The mixture was concentrated and then partitioned between dichoromethane and saturated sodium carbonate solution. The precipitate was collected by filtration and washed with dichloromethane, methanol and water and dried in vacuum oven to give N-(5-(3-amino-1-phenylpropylcarbamoyl)-2-chlorophenyl)-7-methoxy-2-oxo-1,2-dihydro-1,8-naphthyridine-3-carboxamide. (Yield 0.055 g, 72.3%).

HR-MS (ES⁺) m/z Calculated for C₂₆H₂₅ClN₅O₄ ([M+H]⁺): 506.1590. Found: 506.1589.

The pharmacological properties of the compounds of this invention may be confirmed by a number of pharmacological assays. The exemplified antiproliferative activity assays which follow have been carried out with compounds according to the invention.

If test compounds were assessed in multiple runs of the same assay, the activities reported in Table I are the averages of the results obtained from the multiple runs of the assay.

### Example 102

### DYRK1B Kinase TR-FRET (IMAP-Tb) Assay

### Assay Principle

The kinase TR-FRET (IMAP-Tb) assay uses a fluorescence labeled substrate peptide in the kinase reaction. Upon phosphorylation by the kinase, phosphopeptide is produced, which will be detected by the binding solution provided in IMAP TR-FRET binding kit. After the completion of the kinase reaction, the reaction will be stopped by adding the binding solution containing terbium tracer. This tracer is immobilized on the surface of the IMAP beads, which also contain metal ions on the beads that bind to the phospho-groups of the products. Thus the phosphorylated product of the reaction can enter into close proximity to the tracer, producing resonance energy transfer. Due to the long lifetime of terbium (Tb) fluorescence the detection can be run in time resolved mode, which virtually eliminates fluorescence interference from assay components or compounds.

The TR-FRET signal measurement from this assay, given as an IC₅₀ measurement, is a measure of a test compound's ability to interfere with the phosphorylation of the peptide substrate, that is it inhibits the phosphorylation of the substrate peptide by DYRK1B, and is thus a measure of the test compound's ability to inhibit the activity of DYRK1B. IC₅₀ is the amount of test compound that inhibits 50% of the activity of DYRK1B in this assay. In some cases where the IC₅₀ values were not determined, then the % inhibition at 10 μM test compound concentration may be reported instead. The results of this assay for sample compounds of the invention are provided in Table I below.

### Materials and Reagents

1. Human DYRK1B: from Invitrogen. Part #PR8350B (former PV4649)
2. Substrate Peptide: in-house synthesized: RRRFRPASPLRGPPK
3. IMAP TR-FRET IPP Explorer Kit: from Molecular Devices. Part #R8157
4. Kinase Assay Buffer (KAB): 10 mM HEPES pH 7.0, 50 mM NaCl, 5 mM MgCl₂, 1 mM DTT, 1 mM NaVO₄, 200 μg/mL BSA (0.02%)
5. Assay Plate: Remp polypropylene clear 384-well microplate. Cat#23490-102
6. Detection Plate: Costa black 384-well microplate. Cat #3710
   Assay Procedure: This assay was performed as follows:
1. Transfer 1.5 μL of 20× compound solution to each well of an assay plate.
2. Add to each well 22.5 μL of KAB Buffer.
3. Add to each well 3 μL of the solution of DYRK1B and ATP. The final concentration of DYRK1B is 1.25 nM and ATP concentration is 70 μM (3 times of Km of ATP, which is 23.3 μM)
4. Add to each well 3 μL of the substrate peptide. The assay concentration is 1.0 μM
5. Incubate the assay plates at 37° C. for 60 minutes.
6. Add 18 μL of Detection Solution (1:800 diluted Progress bead stock, 1:400 diluted Tb stock, 80% Buffer A, and 20% Buffer B) into each well of detection plates.
7. Transfer 6 μL of assay solution from the assay plate to the detection plate.
8. Shake detection plates for 30 minutes.
9. Read plates in Envision with wavelength set at excitation 340 nm for Tb, emission 490 nm, and excitation 520 nm.
10. Calculation:

TR-FRET Signal=(Reading at 520 nM/Reading at 490 nM)×2000000

### Example 103

### DYRK1A Kinase TR-FRET (IMAP-Tb) Assay

### Assay Principle

The kinase TR-FRET (IMAP-Tb) assay uses a fluorescence labeled substrate peptide in the kinase reaction. Upon phosphorylation by the kinase, phosphopeptide is produced, which will be detected by the binding solution provided in IMAP TR-FRET binding kit. After the completion of the kinase reaction, the reaction will be stopped by adding the binding solution containing terbium tracer. This tracer is immobilized on the surface of the IMAP beads, which also contain metal ions on the beads that bind to the phospho-groups of the products. Thus the phosphorylated product of the reaction can enter into close proximity to the tracer, producing resonance energy transfer. Due to the long lifetime of terbium (Tb) fluorescence the detection can be run in time resolved mode, which virtually eliminates fluorescence interference from assay components or compounds.

The TR-FRET signal measurement from this assay, given as an IC₅₀ measurement, is a measure of a test compound's ability to interfere with the phosphorylation of the peptide substrate, that is it inhibits the phosphorylation of the substrate peptide by DYRK1A, and is thus a measure of the test compound's ability to inhibit the activity of DYRK1A. IC₅₀ is the amount of test compound that inhibits 50% of the activity of DYRK1A in this assay. In some cases where the IC₅₀ values were not determined, then the % inhibition at 10 μM test compound concentration may be reported instead. The results of this assay for sample compounds of the invention are provided in Table I below.

### Materials and Reagents

1. Human DYRK1A: from Invitrogen. Part #PV3997
2. Substrate Peptide: RRRFRPASPLRGPPK
3. IMAP TR-FRET IPP Explorer Kit: from Molecular Devices. Part #R8157
4. Kinase Assay Buffer (KAB): 10 mM HEPES pH 7.0, 50 mM NaCl, 5 mM MgCl₂, 1 mM DTT, 1 mM NaVO₄, 200 μg/mL BSA (0.02%)
5. Assay Plate: Remp polypropylene clear 384-well microplate. Cat#23490-102
6. Detection Plate: Costa black 384-well microplate. Cat #3710
   Assay Procedure: This assay was performed as follows:
1. Transfer 1.5 μL of 20× compound solution to each well of an assay plate.
2. Add to each well 22.5 μL of KAB Buffer.
3. Add to each well 3 μL of the solution of DYRK1A and ATP. The final concentration of DYRK1A is 1.25 nM and ATP concentration is 70 μM (3 times of Km of ATP, which is 23.3 μM)
4. Add to each well 3 μL of the substrate peptide. The assay concentration is 1.0 μM
5. Incubate the assay plates at 37° C. for 60 minutes.
6. Add 18 μL of Detection Solution (1:800 diluted Progress bead stock, 1:400 diluted Tb stock, 80% Buffer A, and 20% Buffer B) into each well of detection plates.
7. Transfer 6 μL of assay solution from the assay plate to the detection plate.
8. Shake detection plates for 30 minutes.
9. Read plates in Envision with wavelength set at excitation 340 nm for Tb, emission 490 nm, and excitation 520 nm.
10. Calculation:

TR-FRET Signal=(Reading at 520 nM/Reading at 490 nM)×2000000

### Example 104

### SW620 Cell Viability Assay

1. Cell Plate Preparation: SW620 human colon cancer cells (known to express DYRK1B), obtained from ATCC, were seeded into 96-well plates at 3×10³ cells/well in 50 μL of media.
Harvested the required number of cells (counts & viability determined by Guava Viacount).
Centrifuge cells to pellet and removed supernant.
Resuspended in growth media (50 μL/well) & pipetted thoroughly to break up clumps.
Setup for ˜100 wells/plate, therefore, V_{T}=5 mL/plate @ 1.2×10⁵ cells/mL.
   1A. Some cells (S—) were allowed to attach for 24 hrs, serum starved for 48 hrs, and then followed with drug treatments.
   Test compound solution were prepared in regular corresponding media supplemented with serum.
   2. Test Compound Preparation: Test compounds were solubilized in either DMSO or media and prepared at various stock concentrations.
All compounds were incubated at 37° C. for 30 minutes and vortexed.
(5 mM stocks of test compound were prepared for non-soluble compounds)

### 3. Drug Plate Preparation:

The 10 mM test compound stock was diluted to a concentration of 100× the final Cₘₐₓ concentration.
Then the test compound stock is diluted 50-fold in media and/or second compound for a final Cₘₐₓ concentration in the test compound plate.
The Cₘₐₓ in the test compound plate (2% DMSO) is 2-fold higher than the final Cₘₐₓ in the cell plate (60 uL titrations were 1:3).
   4. Viability Assay: This assay was performed as follows:
Transfer 50 μL of test compound solution per well from the test compound plate onto the cell plate prepared in Step #1.
Mix the plate with treated cells by pipetting up and down three times with 200 μL multi-channel pipette.
Incubate the cells in 5% CO₂ incubator @ 37° C. for 4 days.
Run the CellTiter-Glo™ Luminescent Cell Viability Assay.

The results of this assay, given as EC₅₀ values, indicate the concentration of test compound that inhibits tumor cell proliferation by 50%. The results of this assay for sample compounds of the invention are provided in Table I below.

TABLE 1
Kinase enzyme and cellular activity

| | Enzyme | Enzyme | Cellular |
|---|---|---|---|
| | IC₅₀ | IC₅₀ | EC₅₀ |
| | (μM) | (μM) | (μM) |
| Example | DYRK1B | DYRK1A | SW620 |
| 28 | >10 | 24% | |
| 30 | 0.119 | 0.135 | >10 |
| 33 | 0.484 | 2.149 | 1.469 |
| 34 | 28% | 45% | |
| 35 | 35% | 45% | |
| 36 | >10 | >10 | |
| 37 | 0.917 | 0.588 | 0.47 |
| 32 | 19% | 23% | |
| 39 | 7.216 | 0.63 | |
| 40 | 10 | 0.051 | |
| 41 | >10 | >10 | |
| 42 | 41% | 27% | |
| 43 | 0.041 | 0.028 | >10 |
| 45 | 0.014 | 0.0115 | 0.1769 |
| 49 | 0.537 | 0.3 | >10 |
| 38 | 0.424 | 0.266 | >10 |
| 51 | 0.119 | 0.0198 | 0.373 |
| 53 | 0.0138 | 0.0046 | 0.0524 |
| 55 | 0.0915 | 0.0506 | 0.68 |
| 57 | 0.146 | 0.0512 | 0.496 |
| 59 | 0.0308 | 0.0113 | 0.299 |
| 61 | 0.155 | 0.034 | 2.269 |
| 63 | 0.023 | 0.0046 | 0.2045 |
| 65 | 0.21 | 0.0974 | 0.915 |
| 67 | 0.0854 | 0.00952 | 0.103 |
| 69 | 0.0137 | 0.0124 | 0.256 |
| 71 | <0.0046 | 0.0046 | 0.137 |
| 73 | 0.96 | 0.425 | >10 |
| 74 | 3.55 | 0.96 | |
| 87 | 1.309 | 0.915 | |
| 88 | 0.513 | 0.451 | 1.53 |
| 89 | 47% | 0.125 | |
| 90 | 0.168 | >10 | >10 |
| 94 | 0.079 | 0.05 | |
| 95 | 0.047 | 0.019 | 1.24 |
| 98 | 0.69 | 0.79 | |
| 99 | 0.235 | 0.123 | >10 |
| 100 | 0.0158 | 0.0162 | |
| 101 | 0.0527 | 0.0392 | 0.0273 |

What is claimed:

1. A compound of formula I 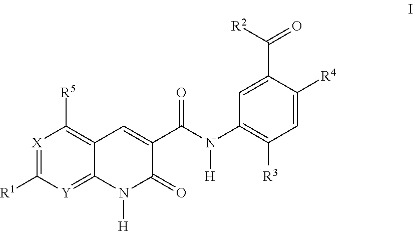
wherein
X and Y are each independently selected from C and N, provided that when one is C and the other is N;
R¹ is selected from the group
(a) H,
(b) C₁₋₄ alkyl,
(c) C₁₋₄ alkyl substituted with up to 3 groups selected from cycloalkyl, heterocycle, OR⁶, NR⁶R⁷, and CN,
(d) OR⁶,
(e) NR⁶R⁷,
(f) heterocycle that is attached to the rest of the molecule via a heteroatom
(g) heterocycle substituted with up to three groups selected from C₁₋₄ alkyl, OR⁸, NR⁸R⁹ and CN, and
(h) SR⁶;
R² is selected from the group
(a) NR¹⁰R¹¹, and
(b) OR¹²;
R³ is selected from the group
(a) CH₃,
(b) F,
(c) Cl, and
(d) Br;
R⁴ is selected from the group
(a) H, and
(b) F;
R⁵ is selected from the group
(a) H, and
(b) C₁₋₄ alkyl
R⁶ and R⁷ are independently selected from the group
(a) H,
(b) C₁₋₄ alkyl,
(c) C₁₋₄ alkyl substituted with up to 3 groups selected from OH, OC₁₋₄ alkyl, NR⁸R⁹, CN, heterocycle, and cycloalkyl,
(d) cycloalkyl,
(e) cycloalkyl substituted with up to 3 groups selected from OH, NR⁸R⁹ and C₁₋₄ alkyl,
(f) heterocycle, and
(g) heterocycle substituted with up to three C₁₋₄ alkyl groups;
R⁸ and R⁹ are independently selected from the group
(a) H, and
(b) C₁₋₄ alkyl;
R¹⁰ and R¹¹ are independently selected from the group
(a) H,
(b) C₁₋₆ alkyl,
(c) C₁₋₆ alkyl substituted with up to 4 groups selected from
aryl,
aryl substituted with Cl, F, or CH₃,
heteroaryl,
cycloalkyl,
heterocycle,
OH,
OC₁₋₄ alkyl,
NR⁸R⁹,
CN; and
CONR⁸R⁹, and
(d) aryl optionally substituted with Cl, F or CH₃;
or alternatively, NR¹⁰R¹¹ together can form a heterocycle that optionally may be substituted with
Cl,
F,
CH₃,
aryl that optionally may be substituted with Cl, F, and CH₃, and
heteroaryl that optionally may be substituted with Cl, F, and CH₃; and
R¹² is selected from the group
(a) C₁₋₆ alkyl,
(b) C₁₋₆ alkyl substituted with up to 4 groups selected from
aryl,
aryl substituted with Cl, F, or CH₃,
heteroaryl,
cycloalkyl,
heterocycle,
OH,
OC₁₋₄ alkyl,
NR⁸R⁹,
CN; and
CONR⁸R⁹, and
(c) aryl optionally substituted with Cl, F or CH₃;
or a pharmaceutically acceptable salt thereof.

2. The compound claim 1 wherein X is N and Y is C, or a pharmaceutically acceptable salt thereof.

3. The compound of claim 1 wherein X is C and Y is N, or a pharmaceutically acceptable salt thereof.

4. The compound of claim 1, wherein R¹ is H, or a pharmaceutically acceptable salt thereof.

5. The compound of claim 1, wherein R¹ is OR⁶ and R⁶ is C₁₋₄ alkyl, or a pharmaceutically acceptable salt thereof.

6. The compound of claim 5 wherein OR⁶ is OCH₃, or a pharmaceutically acceptable salt thereof.

7. The compound of claim 1, wherein R¹ is SR⁶ and R⁶ is C₁₋₄ alkyl, or a pharmaceutically acceptable salt thereof.

8. The compound of claim 7 wherein SR⁶ is SCH₃, or a pharmaceutically acceptable salt thereof.

9. The compound of claim 1, wherein R¹ is heterocycle optionally substituted with C₁₋₄ alkyl, NR⁸R⁹ or OR⁸, and R⁸ and R⁹ are independently selected from H and methyl, or a pharmaceutically acceptable salt thereof.

10. The compound of claim 9 wherein R¹ is heterocycle optionally substituted with CH₃, NH₂ or OH, or a pharmaceutically acceptable salt thereof.

11. The compound of claim 10, wherein R¹ is selected from piperidinyl, piperazinyl and morpholinyl each of which may optionally be substituted with CH₃, or a pharmaceutically acceptable salt thereof.

12. The compound of claim 1, wherein R¹ is NR⁶R⁷ and R⁶ and R⁷ are independently selected from H and C₁₋₄ alkyl that optionally is substituted with OH, OCH₃, heterocycle or cycloalkyl, or a pharmaceutically acceptable salt thereof.

13. The compound of claim 1, wherein R² is OR¹², or a pharmaceutically acceptable salt thereof.

14. The compound of claim 13 wherein R¹² is C₁₋₄ alkyl optionally substituted with aryl or a pharmaceutically acceptable salt thereof.

15. The compound of claim 14 wherein R¹² is methyl, or a pharmaceutically acceptable salt thereof.

16. The compound of claim 14 wherein R¹² is C₁₋₄ alkyl substituted with phenyl which optionally is substituted with Cl, or a pharmaceutically acceptable salt thereof.

17. The compound of claim 1, wherein R² is NR¹⁶R¹¹ and each of R¹⁰ and R¹¹ is independently selected from H and C₁₋₆ alkyl that is optionally substituted with NH₂, OH, CONH₂, cycloalkyl, heterocycle, heteroaryl, or aryl that optionally is substituted with Cl, or a pharmaceutically acceptable salt thereof.

18. The compound of claim 17 wherein one of R¹⁰ or R¹¹ is phenyl that optionally is substituted with Cl, or a pharmaceutically acceptable salt thereof.

19. The compound of claim 17 wherein R¹⁰ and R¹¹ are independently selected from H and C₁₋₆ alkyl that is substituted with NH₂ and thiophene or phenyl, or a pharmaceutically acceptable salt thereof.

20. The compound of claim 1, wherein R³ is C₁ or CH₃, or a pharmaceutically acceptable salt thereof.

21. The compound of claim 1, wherein R⁴ is H, or a pharmaceutically acceptable salt thereof.

22. The compound of claim 1, wherein R⁵ is H, or a pharmaceutically acceptable salt thereof.

23. The compound of claim 1, wherein R⁶ and R⁷ are independently selected from H and C₁₋₄ alkyl that optionally is substituted with OH, OCH₃ or N-ethyl, or a pharmaceutically acceptable salt thereof.

24. The compound of claim 1, wherein R⁸ and R⁹ are independently selected from H and CH₃, or a pharmaceutically acceptable salt thereof.

25. The compound of claim 1, wherein R¹⁹ and R¹¹ are independently selected from H and C₁₋₆ alkyl that optionally is substituted with aryl that is optionally substituted with C₁ or CH₃, or a pharmaceutically acceptable salt thereof.

26. The compound of claim 25 wherein the aryl is phenyl.

27. The compound of claim 1, wherein R¹⁹ and R¹¹ are independently selected from H and C₁₋₆ alkyl that optionally is substituted with heteroaryl, or a pharmaceutically acceptable salt thereof.

28. The compound of claim 27 wherein the heteroaryl is thiophenyl.

29. The compound of claim 1, wherein R¹⁰ and R¹¹ are independently selected from H and C₁₋₆ alkyl that optionally is substituted with OH, OCH₃, NH₂ and CONH₂, or a pharmaceutically acceptable salt thereof.

30. The compound of claim 1, wherein R¹⁰ and R¹¹ are independently selected from H and aryl that optionally may be substituted with Cl, or a pharmaceutically acceptable salt thereof.

31. The compound of claim 30 wherein the aryl is phenyl.

32. The compound of claim 1, wherein R¹² is C₁₋₄ alkyl, or a pharmaceutically acceptable salt thereof.

33. The compound of claim 32 wherein R¹² is CH₃.

34. The compound of claim 1 selected from the group comprising:
3-[(7-Methoxy-2-oxo-1,2-dihydro-[1,6]naphthyridine-3-carbonyl)-amino]-4-methyl-benzoic acid methyl ester;
4-Chloro-3-[(7-methoxy-2-oxo-1,2-dihydro-[1,6]naphthyridine-3-carbonyl)-amino]-benzoic acid methyl ester;
7-Methoxy-2-oxo-1,2-dihydro-[1,6]naphthyridine-3-carboxylic acid (5-benzylcarbamoyl-2-chloro-phenyl)-amide;
7-Methoxy-2-oxo-1,2-dihydro-[1,6]naphthyridine-3-carboxylic acid (5-benzylcarbamoyl-2-methyl-phenyl)-amide;
7-Methoxy-2-oxo-1,2-dihydro-[1,6]naphthyridine-3-carboxylic acid [5-(2-chloro-benzylcarbamoyl)-2-methyl-phenyl]-amide;
7-Methoxy-2-oxo-1,2-dihydro-[1,6]naphthyridine-3-carboxylic acid [5-(3-chloro-benzylcarbamoyl)-2-methyl-phenyl]-amide;
7-Methoxy-2-oxo-1,2-dihydro-[1,6]naphthyridine-3-carboxylic acid [5-(4-chloro-benzylcarbamoyl)-2-methyl-phenyl]-amide;
7-Methoxy-2-oxo-1,2-dihydro-[1,6]naphthyridine-3-carboxylic acid {2-methyl-5-[(thiophen-2-ylmethyl)-carbamoyl]-phenyl}-amide;
7-Methoxy-2-oxo-1,2-dihydro-[1,6]naphthyridine-3-carboxylic acid {2-methyl-5-[(thiophen-3-ylmethyl)-carbamoyl]-phenyl}-amide; and
7-Methoxy-2-oxo-1,2-dihydro-[1,6]naphthyridine-3-carboxylic acid [2-chloro-5-(2-chloro-benzylcarbamoyl)-phenyl]-amide;
or a pharmaceutically acceptable salt of any of the foregoing compounds.

35. The compound of claim 1 selected from the group comprising:
7-Methoxy-2-oxo-1,2-dihydro-[1,6]naphthyridine-3-carboxylic acid [2-chloro-5-(3-chloro-benzylcarbamoyl)-phenyl]-amide;
7-Methoxy-2-oxo-1,2-dihydro-[1,6]naphthyridine-3-carboxylic acid [2-chloro-5-(4-chloro-benzylcarbamoyl)-phenyl]-amide;
7-Methoxy-2-oxo-1,2-dihydro-[1,6]naphthyridine-3-carboxylic acid {2-chloro-5-[(thiophen-2-ylmethyl)-carbamoyl]-phenyl}-amide;
7-Methoxy-2-oxo-1,2-dihydro-[1,6]naphthyridine-3-carboxylic acid {2-chloro-5-[(thiophen-3-ylmethyl)-carbamoyl]-phenyl}-amide;
7-Methoxy-2-oxo-1,2-dihydro-[1,6]naphthyridine-3-carboxylic acid [5-(2-amino-1-phenyl-ethylcarbamoyl)-2-chloro-phenyl]-amide; hydrochloride;
3-{[7-(2,3-Dihydroxy-propylamino)-2-oxo-1,2-dihydro-[1,6]naphthyridine-3-carbonyl]-amino}-4-methyl-benzoic acid methyl ester; hydrochloride;
7-Methoxy-2-oxo-1,2-dihydro-[1,6]naphthyridine-3-carboxylic acid {5-[2-amino-1-(3-chloro-phenyl)-ethylcarbamoyl]-2-chloro-phenyl}-amide; hydrochloride;
7-Methoxy-2-oxo-1,2-dihydro-[1,6]naphthyridine-3-carboxylic acid [5-(3-amino-1-thiophen-3-yl-propylcarbamoyl)-2-chloro-phenyl]-amide;
7-Methoxy-2-oxo-1,2-dihydro-[1,6]naphthyridine-3-carboxylic acid [5-(2-amino-1-phenyl-ethylcarbamoyl)-2-methyl-phenyl]-amide; hydrochloride; and
7-Methoxy-2-oxo-1,2-dihydro-[1,6]naphthyridine-3-carboxylic acid {5-[2-amino-1-(3-chloro-phenyl)-ethylcarbamoyl]-2-methyl-phenyl}-amide; hydrochloride;
or a pharmaceutically acceptable salt of any of the foregoing compounds.

36. The compound of claim 1 selected from the group comprising:
7-Methoxy-2-oxo-1,2-dihydro-[1,6]naphthyridine-3-carboxylic acid [5-(2-amino-1-thiophen-3-yl-ethylcarbamoyl)-2-methyl-phenyl]-amide; hydrochloride;
7-Methoxy-2-oxo-1,2-dihydro-[1,6]naphthyridine-3-carboxylic acid [5-(3-amino-1-phenyl-propylcarbamoyl)-2-methyl-phenyl]-amide; hydrochloride;
7-Methoxy-2-oxo-1,2-dihydro-[1,6]naphthyridine-3-carboxylic acid [5-(3-amino-1-thiophen-3-yl-propylcarbamoyl)-2-methyl-phenyl]-amide;
7-Methoxy-2-oxo-1,2-dihydro-[1,6]naphthyridine-3-carboxylic acid {5-[3-amino-1-(3-chloro-phenyl)-propylcarbamoyl]-2-methyl-phenyl}-amide; hydrochloride;
7-Methoxy-2-oxo-1,2-dihydro-[1,6]naphthyridine-3-carboxylic acid {5-[3-amino-1-(3-chloro-phenyl)-propylcarbamoyl]-2-chloro-phenyl}-amide; hydrochloride;
7-Methoxy-2-oxo-1,2-dihydro-[1,6]naphthyridine-3-carboxylic acid [5-(3-amino-1-phenyl-propylcarbamoyl)-2-chloro-phenyl]-amide; hydrochloride;
7-Methoxy-2-oxo-1,2-dihydro-[1,6]naphthyridine-3-carboxylic acid [5-(2-amino-1-thiophen-3-yl-ethylcarbamoyl)-2-chloro-phenyl]-amide; hydrochloride;
4-Chloro-3-{[7-(4-methyl-piperazin-1-yl)-2-oxo-1,2-dihydro-[1,6]naphthyridine-3-carbonyl]-amino}-benzoic acid methyl ester;
4-Methyl-3-{[7-(4-methyl-piperazin-1-yl)-2-oxo-1,2-dihydro-[1,6]naphthyridine-3-carbonyl]-amino}-benzoic acid methyl ester; and
4-Methyl-3-{[7-(4-methyl-piperazin-1-yl)-2-oxo-1,2-dihydro-[1,8]naphthyridine-3-carbonyl]-amino}-benzoic acid methyl ester;
or a pharmaceutically acceptable salt of any of the foregoing compounds.

37. The compound of claim 1 selected from the group comprising:
4-Chloro-3-{[7-(4-methyl-piperazin-1-yl)-2-oxo-1,2-dihydro-[1,8]naphthyridine-3-carbonyl]-amino}-benzoic acid methyl ester;
3-[(7-Methoxy-2-oxo-1,2-dihydro-[1,8]naphthyridine-3-carbonyl)-amino]-4-methyl-benzoic acid methyl ester;
4-Chloro-3-[(7-methoxy-2-oxo-1,2-dihydro-[1,8]naphthyridine-3-carbonyl)-amino]-benzoic acid methyl ester;
N-(5-(Benzylcarbamoyl)-2-chlorophenyl)-7-methoxy-2-oxo-1,2-dihydro-1,8-naphthyridine-3-carboxamide;
N-(2-Chloro-5-(3-chlorobenzylcarbamoyl)phenyl)-7-methoxy-2-oxo-1,2-dihydro-1,8-naphthyridine-3-carboxamide;
N-(5-(Benzylcarbamoyl)-2-methylphenyl)-7-methoxy-2-oxo-1,2-dihydro-1,8-naphthyridine-3-carboxamide;
N-(5-(3-Chlorobenzylcarbamoyl)-2-methylphenyl)-7-methoxy-2-oxo-1,2-dihydro-1,8-naphthyridine-3-carboxamide;
N-(5-(3-Amino-1-phenylpropylcarbamoyl)-2-methylphenyl)-7-methoxy-2-oxo-1,2-dihydro-1,8-naphthyridine-3-carboxamide; and
N-(5-(3-Amino-1-phenylpropylcarbamoyl)-2-chlorophenyl)-7-methoxy-2-oxo-1,2-dihydro-1,8-naphthyridine-3-carboxamide;
or a pharmaceutically acceptable salt of any of the foregoing compounds.

38. A pharmaceutical composition comprising a compound of claim 1, or a pharmaceutically acceptable salt thereof, as an active ingredient and a pharmaceutically acceptable carrier or excipient.
